(19) United States
(12) Patent Application Publication
(10) Pub. No.: US 2025/0154462 A1
(43) Pub. Date: May 15, 2025

(54) COMPOSITIONS AND METHODS FOR EXPANDING IMMUNE CELLS

(71) Applicant: Rutgers, The State University of New Jersey, New Brunswick, NJ (US)

(72) Inventors: Dongfang Liu, Millburn, NJ (US); Xuening Wang, Old Bridge, NJ (US)

(73) Assignee: Rutgers, The State University of New Jersey, New Brunswick, NJ (US)

(21) Appl. No.: 18/832,699

(22) PCT Filed: Jan. 26, 2023
(86) PCT No.: PCT/US2023/061346

§ 371 Date: Jul. 24, 2024

Related U.S. Application Data
(60) Provisional application No. 63/303,356, filed on Jan. 26, 2022.

(51) Int. Cl. C12N 5/0783; A61K 40/15; A61K 40/31; A61K 40/42

(52) U.S. Cl. CPC C12N 5/0646; A61K 40/15; A61K 40/31; A61K 40/4211; C12N 2501/2302; C12N 2501/2315; C12N 2501/2321; C12N 2502/1107; C12N 2510/00

(57) ABSTRACT

Methods of expanding a population of immune cells, such as natural killer cells or CAR-modified natural killer cells, using exosomes, and methods of producing the exosomes from a population of 721.221 cells transduced or transfected with a membrane-bound IL-21 (mIL-21) or a population of 721.22 cells transduced or transfected with a mIL-21 and B7-H6. Methods of treating a cancer or an infectious or immune disease, wherein immune cells expanded using the exosomes are administered to a subject with the cancer or the infectious or immune disease are also provided.

### CROSS REFERENCE TO RELATED APPLICATION

This application claims the benefit of U.S. Provisional Application No. 63/303,356, filed Jan. 26, 2022, which is incorporated herein by reference in its entirety.

### ACKNOWLEDGMENT OF GOVERNMENT SUPPORT

This invention was made with government support under grant number 1R01AI130197-01A1 awarded by the National Institutes of Health. The government has certain rights in the invention.

### FIELD

This disclosure relates to methods of expanding immune cells, including expanding immune cells in the absence of feeder cells, methods of producing exosomes, methods of using the exosomes to expand immune cells, and methods of using the immune cells.

### INCORPORATE OF SEQUENCE LISTING

The Sequence Listing is submitted as an XML file in the form of the file named 7213-107775-02_Sequence_Listing.xml, which was created on Jan. 19, 2023, and is 24,749 bytes, which is incorporated by reference herein in its entirety.

### BACKGROUND

Immunotherapies have revolutionized the treatment of cancer. One such therapy engineers immune cells to express chimeric antigen receptors (CARs) that both recognize tumor antigens and activate immune cells. The adoptive transfer of CAR-modified immune cells (especially CAR-T cells) into patients has been successful in treating refractory blood cancers. Because CAR-T cell therapies are associated with toxic side effects (e.g., cytokine storms, neurotoxicity, and cardiotoxicity), CARs are being developed for use in human natural killer (NK) cells. CAR-NK cells exhibit lower toxicities and greater ‘off-the-shelf’ potential as compared to CAR-T cells. However, currently available technologies for ex-vivo expansion of NK and CAR-NK cells use feeder cells expressing membrane-bound forms of co-stimulatory molecules (e.g., IL-15, IL-21, and others), and have several limitations. Specifically, NK cells expanded with feeder cell systems (such as K562 cells) have limited life-spans due to telomere shortage, exhaustion, and fratricidal killing.

### SUMMARY

There remains a need for improved cytotoxic cell-mediated immunotherapies, for example, to expand immune cells (such as NK and T cells) in the absence of feeder cells, for use in immunotherapeutic applications. Disclosed herein are methods of expanding a population of immune cells (such as NK cells, T cells, NKT cells, and macrophages) using a non-feeder cell expansion system. The methods include contacting the population of immune cells with an exosome isolated from a population of 721.221 cells transduced or transfected with a nucleic acid encoding membrane-bound IL-21 (mIL-21), under conditions sufficient for cell expansion. In some embodiments, the nucleic acid encoding mIL-21 further includes additional immune cell regulatory components, such as at least portions from the IgG1 CH2-CH3 domain, T cell surface glycoprotein CD3 zeta chain (CD3ζ), 4-1BB, CD28, or a combination thereof.

In additional embodiments, the exosomes are isolated from a population of 721.221 cells transduced or transfected with a nucleic acid encoding membrane-bound IL-21 (mIL-21) and a nucleic acid encoding B7-H6 (referred to as 721.221-mIL21-B7H6 cells). In some embodiments, the methods include contacting a population of immune cells with an exosome isolated from a population of 721.221 cells transduced or transfected with a nucleic acid encoding membrane-bound IL-21 (mIL-21) and a nucleic acid encoding B7-H6, under conditions sufficient for cell expansion.

Also disclosed are isolated exosomes and methods of producing the isolated exosomes for use in the disclosed methods of expanding a population of immune cells. In some embodiments the methods include isolating exosomes from a population of 721.221-mIL-21 cells. In some embodiments, the exosomes are isolated from a supernatant from a culture of 721.221-mIL-21 cells, such as by centrifugation, such as by serial ultracentrifugation. Also provided are exosomes isolated from a population of 721.221-mIL21-B7H6 cells. In some embodiments the methods include isolating exosomes from a population of 721.221-mIL21-B7H6 cells. In some embodiments, the exosomes are isolated from a supernatant from a culture of 721.221-mIL21-B7H6 cells, such as by centrifugation, such as by serial ultracentrifugation.

Further disclosed are methods of treating a cancer or an infectious or immune disease, including administering the immune cells expanded according to the disclosed methods to a subject with the cancer or the infectious or immune disease. In some embodiments, the method includes contacting the population of immune cells with isolated exosomes produced according to the disclosed methods, thereby expanding the population of immune cells.

In some embodiments of the disclosed methods, the immune cells are NK cells, T cells, macrophages, or NKT cells. In some embodiments, the population of immune cells is further treated with at least one cytokine, such as at least one interleukin, such as IL-2 and/or IL-15. In some embodiments, the population of 721.221-mIL-21 cells is treated with one or more toll-like receptor (TLR) ligands, prior to isolating the exosomes.

The 721.221 cells of the disclosed methods may be transduced with a nucleic acid encoding mIL-21, B7-H6, or both, using a viral vector, such as a retroviral vector, such as a Moloney murine leukemia virus (MoMLV) vector, such as an SFG retroviral vector. In some embodiments, the 721.221-mIL-21 cells, 721.221-B7H6, or 721.221-mIL21-B7H6 cells are further transduced or transfected with a nucleic acid encoding an additional heterologous cytokine, activating receptor ligand, TLR ligand, or receptor thereof, and/or IL-15Rα.

The disclosed immune cells may be from peripheral blood, cord blood, ascites, menstrual blood, or bone marrow, such as peripheral blood mononuclear cells (PBMCs) or purified NK cells. The immune cells may be autologous to the subject. In some embodiments, the disclosed immune cells are modified cells, such as CAR-modified NK cells, CAR-modified T cells, CAR-modified macrophages, or CAR-modified NKT cells. In some embodiments, the CAR-NK cell is a CD19 CAR-NK cell or a CD147 CAR-NK cell.

Also provided are modified 721.221 cells expressing at least membrane-bound IL-21 (mIL-21) and B7-H6. In specific, non-limiting examples, the modified 721.221 cells express mIL-21, such as an amino acid sequence with at least 90% or 95% sequence identity to SEQ ID NO: 2 (and/or as encoded by a nucleic acid sequence with at least 90% or 95% sequence identity to SEQ ID NO: 1), and B7-H6, such as an amino acid sequence with at least 90% or 95% sequence identity to SEQ ID NO: 12 (and/or as encoded by a nucleic acid sequence with at least 90% or 95% sequence identity to SEQ ID NO: 11). In some examples, the mIL-21 and B7-H6 are expressed using one or more viral vectors (such as one or more retroviral vectors, e.g., a lentivirus, such as a Moloney murine leukemia virus (MoMLV) vector, such as an SFG retroviral vector).

Further disclosed herein are methods of producing modified 721.221 cells expressing mIL-21 and B7-H6, for example, including transducing or transfecting a population of 721.221 cells with a nucleic acid encoding mIL-21 and a nucleic acid encoding B7-H6; isolating the cells that express mIL-21 and B7-H6; and irradiating the isolated cells, thereby producing the modified 721.221-mIL21-B7H6 cells. In some examples, the cells are modified through transduction (e.g., using a viral vector such as a retrovirus or a lentivirus).

Also disclosed herein are methods of expanding a population of natural killer (NK) cells or T cells, for example, by contacting a population of lymphocytes with a modified 721.221 cell expressing mIL-21 and B7-H6 and at least one cytokine (e.g., an interleukin, such as IL-15 or IL-2) for 1-40 (e.g., 14-21 days) days under conditions sufficient for cell expansion. The population of lymphocytes can be from any sample type, such as peripheral blood, cord blood, ascites, menstrual blood, or bone marrow, and can, for example, include peripheral blood mononuclear cells (PBMCs). The population of cells contacted with the modified 721.221 cells can further include modified cells for immunotherapies, such as chimeric antigen receptor (CAR)-modified cells (e.g., CAR-NK or CAR-T cells, such as CD19 CAR-modified NK cells).

Additionally disclosed herein are methods of treating a cancer or an infectious or immune disease, for example, by administering the NK cells or T cells (e.g., CAR-modified NK or T cells, such as CD19 CAR-modified NK cells) produced using the modified 721.221-IL21-B7H6 cells to a subject with cancer or an infectious or immune disease, thereby treating the cancer or immune disease.

The foregoing and other features of the disclosure will become more apparent from the following detailed description, which proceeds with reference to the accompanying figures.

### BRIEF DESCRIPTION OF THE DRAWINGS

FIG. 1 shows a schematic of the mIL-21-encoding SFG retroviral vector used herein to transfect 721.221 cells. The SFG extracellular domains (comprising mIL-21 and the IgG1 CH2-CH3 domain), transmembrane domain (comprising the CD28 transmembrane domain), and intracellular domains (comprising the CD28 intracellular domain, 4-1BB intracellular domain, and CD3ζ intracellular domain) are shown.

FIG. 2 shows an exemplary protocol illustrating exosome isolation from cell culture medium using an ultracentrifugation method.

FIG. 3 shows exosomes visualized using a confocal microscope. 721.221-mIL-21 cell culture supernatant was isolated using ultracentrifugation and visualized using a confocal microscope, with or without DAPI staining. Culture medium with 10% FBS was used as a control.

FIG. 4 shows mIL-21 expression in naïve 721.221 cells, 721.221-mIL-21 cells, naïve K562 cells, and unstained (US) control cells.

FIGS. 5A-5B show NK cell expansion from peripheral blood mononuclear cells (PBMCs) using the non-feeder cell (NFC)-based NK expansion system compared with a 721.221-mIL-21 feeder cell (FC) expansion system. Peripheral blood NK (PBNK) cells were expanded from a starting population of 5 million PBMCs treated with IL-2 and IL-15, the NFC expansion system, or a FC expansion system (control cells were unstained (US)). FIG. 5A shows a microscopic analysis of the three treatment groups on day 17 of NK cell expansion. Both NF- and FC-based expansion systems resulted in greater expanded NK populations compared to cytokines alone. FIG. 5B shows representative flow cytometry plots of the purities of NK cells expanded with cytokines, the NFC system, or the FC system on day 17 of cell expansion.

FIGS. 6A-6B show NK cell expansion from purified NK cells (pNKs) using the NFC-based NK expansion system compared with a 721.221-mIL-21 FC expansion system. NK cells were expanded from a starting population of 5 million purified NK cells treated with US, IL2 and IL15, the NFC expansion system, or an FC expansion system. FIG. 6A shows a microscopic analysis of the three treatment groups on day 10 of cell expansion. Both NFC- and FC-based expansion systems resulted in greater expanded NK populations compared to cytokines alone. FIG. 6B shows representative flow cytometry plots of the purities of NK cells expanded with cytokines, the NFC system, or an FC system on day 10 of cell expansion.

FIGS. 7A-7D show NK cell expansion profiles using different approaches. FIG. 7A shows fold increase of NK cells from PBMCs (Donors 22 and 23) using cytokines, the NFC system, or an FC system for 28 days. FIG. 7B shows the purities of NK cells expanded from PBMCs (Donor 19) using cytokines, the NFC system, or an FC system for 28 days. FIG. 7C shows fold increase of NK cells from pNKs (Donor 23) using cytokines, the NFC system, or an FC system for 21 days. FIG. 7D shows purities of NK cells expanded from pNKs (Donors 22 and 24) using cytokines, the NFC system, or an FC system for 21 days.

FIGS. 8A-8G show NK cell expansion from PBMCs using the NFC-based NK expansion system compared with a 721.221-mIL-21 FC expansion system for Donor 19. Peripheral blood NK (PBNK) cells were expanded from a starting population of 5 million PBMCs treated with IL-2 and IL-15, the NFC expansion system, or a FC expansion system (control cells were unstained (US)). FIGS. 8A-8B show microscopic analyses of the treatment groups on days 4, 7, 12, and 17 of cell expansion. Membrane protein (MP) was isolated from feeder cells and added into the PBMC-NFC group on day 5 of cell expansion. Both NFC- and FC-based expansion systems resulted in greater expanded NK populations compared to cytokines alone. FIGS. 8C-8G show flow cytometry plots of the purities of NK cells expanded with cytokines, the NFC system, or the FC system on days 7, 12, 17, 24, 28, and 33. The majority of cells in the NFC group were CD3⁺ T cells on day 7 of cell expansion (FIG. 8C). On day 7, NFC-expanded NK cells were separated into two groups. Crude membrane proteins (MP) from 721.221-mIL-21 cells were isolated and added into the culture for one group. NK expansion was increased in the MP-untreated group, while NK expansion was reduced in the MP-treated group (FIG. 8D). NK cells were continuing to increase in number on day 17 (FIG. 8E). On day 14, additional exosomes were added to a portion of the NFC group cells (NF+NF), which reduced NK expansion (% NK cells) compared to the NFC group without additional exosomes (FIG. 8F). The percentage of NK cells in the NFC expanded group continued to increase to 40% (day 28) and 46% (day 33) (FIG. 8G). In contrast, the majority of NK cells expanded using feeder cells began to die on day 33.

FIGS. 9A-9L show NK cell expansion from PBMCs (isolated from donor buffy coat) or from pNKs (isolated from donor PMBCs using the EASYSEP™ Human T Cell Isolation Kit (STEMCELL™ technologies, Cambridge, MA)) using the NFC-based NK expansion system compared with a 721.221-mIL-21 FC expansion system for Donor 22. NK cells were expanded from a starting population of either 1 million PBMCs or 1 million pNK cells. PMBCs or pNKs were treated with IL-2 and IL-15, the NFC expansion system, or a FC expansion system (control cells were unstained (US)). The purity of pNK cells isolated from PBMCs was determined by measuring CD56⁺ and CD3⁻ cells (FIG. 9A). FIGS. 9B-9H show microscopic analyses of the treatment groups on days 3, 7, 14, 17, 21, 28, and 32 of cell expansion. The majority of control pNK cells (IL-2+IL-15 only) had died on day 21 (FIG. 9F). FIGS. 9I-9L show flow cytometry plots of the purities of NK cells expanded from PBMCs or pNKs using cytokines, the NFC system, or the FC system on days 7, 12, 17, and 21. At the beginning of the expansion (day 7), NK percentages for this donor were relatively low in the NFC treatment group (FIG. 91).

FIGS. 10A-10L show NK cell expansion from PBMCs (isolated from donor buffy coat) or from pNKs (isolated from donor PMBCs using the EASYSEP™ Human T Cell Isolation Kit (STEMCELL™ technologies, Cambridge, MA)) using the NFC-based NK expansion system compared with a 721.221-mIL-21 FC expansion system for Donor 23. NK cells were expanded from a starting population of either 5 million PBMCs or 5 million pNK cells. PMBCs or pNKs were treated with IL-2 and IL-15, the NFC expansion system, or a FC expansion system (control cells were unstained (US)). The purity of pNK cells isolated from PBMCs was determined by measuring CD56⁺ and CD3⁻ cells (FIG. 10A). NK cells yield was relatively high for this buffy coat, which also contained a small portion of CD3⁺ cells. FIGS. 10B-10H show microscopic analyses of the treatment groups on days 3, 7, 10, 14, 17, 21, and 28 of cell expansion. FIGS. 10I-10L show flow cytometry plots of the purities of NK cells expanded from PBMCs or pNKs using cytokines, the NFC system, or the FC system on days 7, 10, 14, and 28.

FIGS. 11A-11H show NK cell expansion from PBMCs (isolated from donor buffy coat) or from pNKs (isolated from donor PMBCs using the EASYSEP™ Human T Cell Isolation Kit (STEMCELL™ technologies, Cambridge, MA)) using the NFC-based NK expansion system compared with a 721.221-mIL-21 FC expansion system for Donor 24. NK cells were expanded from a starting population of either 3 million PBMCs or 3 million pNK cells. PMBCs or pNKs were treated with IL-2 and IL-15, the NFC expansion system, or a FC expansion system (control cells were unstained (US)). The purity of pNK cells isolated from PBMCs was determined by measuring CD56⁺ and CD3⁻ cells (FIG. 11A). pNK cell purity with this buffy coat was 95.9%. FIGS. 1IB-11D show microscopic analyses of the treatment groups on days 3, 7, and 14 of cell expansion. FIGS. 11E-11H show flow cytometry plots of the purities of NK cells expanded from PBMCs or pNKs using cytokines, the NFC system, or the FC system on days 3, 7, 14, and 17.

FIGS. 12A-12B show phenotypes of NK cells expanded using different systems. FIG. 12A shows representative histograms of CD16, NKp46, CD94, CD8a, and NKG2C expression in PBNK or pNK cells expanded using cytokines, the NFC system, or a 721.221-mIL-21 FC system, respectively. The median fluorescence intensity (MFI) is noted in the histograms. FIG. 12B shows representative histograms of NKG2A, CTLA-4, KLRG1, PD-1, TIM-3, TIGIT, LAG-3, and KIR2DL1 expression in PBNK or pNK cells expanded using cytokines, the NFC system, or a 721.221-mIL-21 FC system, respectively. The MFI is noted in the histograms.

FIGS. 13A-13B show increased K562 killing activity of PBNKs expanded by different methods. FIG. 13A shows ⁵¹Cr release assay results using PBNK cells expanded from PBMCs using the NFC system or a 721.221-mIL-21 FC system. FIG. 13B shows ⁵¹Cr release assay results using NK cells expanded from pNKs using the NFC system or a 721.221-mIL-21 FC system. Experiments were performed in triplicate. *p<0.05, **p<0.01.

FIGS. 14A-14B show increased CD107a degranulation and K562 cell killing by PBNKs. FIG. 14A shows representative dot plots of CD107a assays with PBMC expended using the NFC system. Over 60% of NFC system-expanded NK cells exhibited degranulation on exposure to K562 cells. FIG. 14B shows the percentage and MFI of CD107a-positive PBNKs.

FIGS. 15A-15B show expression of CD147-CAR in NK cells using the different methods. FIG. 15A shows CD56 and CD3 expression measured using flow cytometry. FIG. 15B shows CD147 CAR expression measured using flow cytometry. CD147-CAR cells were harvested and stained with anti-CD56 and CAR F(ab)2 domain IgG(H+L) for flow cytometry.

FIGS. 16A-16B show the flow cytometry of NK percentage in PBMC and Diagram of PBNK and CAR-NK expansion protocol. FIG. 16A shows the flow cytometry of NK purity in fresh isolated PBMC (Donor 32). FIG. 16B shows the protocol for NK and CAR-NK expansion. Feeder cells 721.221.mIL21 were irradiated with a dose of 100 Gy (10,000 rad), and then PBMCs were co-cultured with irradiated feeder cells with IL-2 and IL-15 for PBNK cell expansion. In parallel, CAR retrovirus was produced by transfecting into 293T cells. The expanded NK cells were transduced with CAR retrovirus at day 4 to day 7. Cells were further cultured in G-Rex, then expanded PBNK and CAR-NK cells were subjected to various functional assays.

FIG. 17 shows NK cell expansion from PBMCs (isolated from donor buffy coat) using the NFC-based NK expansion system compared with a 721.221-mIL-21 FC expansion system for Donor 32.

FIG. 18 shows flow cytometry plots of the purities of NK cells expanded from PBMC from donor 32 using the NFC system or the FC system on days 5, 7, 10, 14, and 20.

FIGS. 19A-19B show purity of NK cell expansion (FIG. 19A) or fold-increase of PBNK from PBMC (FIG. 21B) from donor 32 using the NFC system or the FC system.

FIGS. 20A-20B show killing activity of PBNK expanded from donor 32 with FC system or NFC system by CD107a degranulation assay (FIG. 20A) or ⁵¹Cr killing assay (FIG. 20B).

FIG. 21 shows NK cell expansion from PBMCs of donor 35 using the FC system or the indicated amounts of the NFC system.

FIGS. 22A-22B show flow cytometry on days 7, 10, and 17 of B7H6-221-mIL21 cells by the PE-anti-B7H6 antibody. FIG. 22A is the control cell line 221-mIL21. FIG. 22B is the B7H6-221-mIL21 cell line.

FIGS. 23A-23C show PBNK expansion using co-culture with 221-mIL21 (culturing by MM/or CC as controlling; MM and CC refer to two different batches of 721.221 cells; FIGS. 23A and 23B) or B7H6-221-mIL21 cells (FIG. 23C) on day 3.

FIGS. 24A-24C show PBNK expansion using co-culture with 221-mIL21 (culturing by MM/or CC as controlling; FIGS. 24A and 24B) or B7H6-221-mIL21 cells (FIG. 24C) on day 7.

FIGS. 25A-25C show sorting results of newly-made B7H6-221-mIL21 cells using PE-antiB7H6 antibody (FIG. 24A). The PE-Iso antibody (FIG. 25B) and 221-mIL21 cells (FIG. 25C) were used as controls.

FIG. 26 shows PBNK expansion at day 3 using co-culture with B7H6-221-mIL21 cells.

FIGS. 27A-27C shows flow cytometry of PBMC expansion at day 6 with different ratios of PBMC to feeder cells. Ratios were 1:0.5 (FIG. 27A), 1:1 (FIG. 27B), and 1:2 (FIG. 27C) 221-mIL-21 cells (left) or B7H6-221-mIL21 cells (right) to PBMC.

FIGS. 28A-28C show sorting results of transduced B7H6 alone (FIG. 28A), mIL21 alone (FIG. 28B), or B7H6 and mIL21 (right) on 221-millipore cells (721.221 cells from EMD Millipore). PE-antiB7H6 and APC-antiIL21 antibodies were used.

FIGS. 29A and 29B show flow cytometry of B7H6-mIL21-221 millipore cells 7 days after sorting. PE-antiB7H6 and APC-antiIL21 antibodies were used. These are the same cells in FIG. 28, sorted cells were further culture for 7 days, then flow cytometry was performed to verify the expression of mIL21 and B7H6 in these sorted cells, the flow data confirmed expression of both markers.

### SEQUENCE LISTING

The nucleic acid sequences and amino acid sequences listed in the accompanying sequence listing are shown using standard letter abbreviations for nucleotide bases and amino acids as defined in 37 C.F.R. 1.822. Only one strand of each nucleic acid sequence is shown, but the complementary strand is understood as included by any reference to the displayed strand.

SEQ ID NO: 1 is an exemplary nucleic acid sequence of the extracellular domain from interleukin (IL)-21.

SEQ ID NO: 2 is an exemplary amino acid sequence of the extracellular domain from IL-21.

SEQ ID NO: 3 is an exemplary nucleic acid sequence of a construct for transducing cells with membrane-bound (m)IL-21.

SEQ ID NO: 4 is an exemplary nucleic acid sequence of IL-15Rα.

SEQ ID NO: 5 is an exemplary amino acid sequence of IL-15Rα.

SEQ ID NO: 6 is an exemplary nucleic acid sequence of IL-15.

SEQ ID NO: 7 is an exemplary amino acid sequence of IL-15.

SEQ ID NO: 8 is an exemplary nucleic acid sequence of IL-2.

SEQ ID NO: 9 is an exemplary amino acid sequence of IL-2.

SEQ ID NO: 10 is an exemplary amino acid sequence of a construct for transducing cells with mIL-21.

SEQ ID NO: 11 is an exemplary nucleic acid sequence encoding B7-H6.

SEQ ID NO: 12 is an exemplary B7-H6 amino acid sequence.

SEQ ID NOs: 13 and 14 are forward and reverse primers, respectively, used for preparing B7-H6 vector.

### DETAILED DESCRIPTION

Disclosed herein are methods of expanding a population of immune cells using exosomes isolated from a population of 721.221 cells transduced or transfected with a nucleic acid encoding membrane-bound IL-21 (mIL-21) (“721.221-mIL-21” cells). Also disclosed are isolated exosomes and methods of producing the isolated exosomes from the population of 721.221-mIL-21 cells. The expanded immune cells may be modified cells, such as CAR-modified immune cells. In some embodiments the expanded immune cells or CAR-modified immune cells are used in disclosed methods of treating a cancer or an infectious or immune disease in a subject. The immune cells can be NK cells, T cells, macrophages, or NKT cells. While NK cells and T cells are primarily discussed herein, it is understood that the disclosed methods apply equally to other types of immune cells, including macrophages and NKT cells.

Current methods of expanding immune cell populations (such as NK and T cell populations) using feeder cells (FCs) are associated with safety concerns in human subjects and are therefore subject to strict regulation by the U.S. Food and Drug Administration (FDA) (e.g., Fang et al., Cancer Biol Med. 16(4):647-654, 2019; Lee, Methods Mol Biol. 1441:347-61, 2016; Liu et al., J Hematol Oncol. 14(1):7, 2021). FCs, including K562 and 721.221 cells, are genetically modified cancer cell lines. Usually, FCs are irradiated before co-culturing with immune cell populations (such as NK or T cells) to be expanded. Nonetheless, regulations limit the clinical applications of immune cells expanded using FC systems. Described herein is a novel non-feeder cell (NFC) system that uses exosomes isolated from 721.221-mIL-21 cell cultures (such as from supernatants of TLR ligand-treated 721.221-mIL-21 cells) to expand immune cells. The disclosed NFC system expanded populations of NK cells at rates similar to those of a FC system. The NFC system-expanded populations of NK cells exhibited strong cytotoxicity towards tumor cells. The disclosed NFC system thus expands immune cells in the absence of feeder cells, avoiding the potential safety and regulatory concerns of FC expansion systems.

### I. Terms

Unless otherwise noted, technical terms are used according to conventional usage. Definitions of common terms in molecular biology can be found in Krebs et al. (Eds.), Lewin's Genes XII, published by Jones & Bartlett Publishers, 2017; and Meyers et al. (Eds.), The Encyclopedia of Cell Biology and Molecular Medicine, published by Wiley-VCH in 16 volumes, 2008; and other similar references.

The singular forms “a,” “an,” and “the” refer to one or more than one, unless the context clearly dictates otherwise. For example, the term “comprising an interleukin” includes single or plural interleukins and is considered equivalent to the phrase “comprising at least one interleukin.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank® Accession numbers. In case of conflict, the present specification, including explanations of terms, will control. In addition, the materials, methods, and examples are illustrative only and not intended to be limiting.

To facilitate review of the various embodiments of this disclosure, the following explanations of specific terms are provided.

4-1BB: A member of the TNF-receptor superfamily that contributes to the clonal expansion, survival, and development of T cells. Also known as tumor necrosis factor receptor superfamily member 9 (TNFRSF9). 4-1BB can also induce proliferation in peripheral monocytes, enhance T cell apoptosis induced by TCR/CD3-triggered activation, and regulate CD28 co-stimulation to promote Th1 cell responses. 4-1BB expression is induced by lymphocyte activation. TRAF adaptor proteins have been shown to bind to this receptor and transduce the signals leading to activation of NF-κB. 4-1BB ligand (4-1BBL) and its receptor, 4-1BB, are involved in the antigen presentation process and in the generation of cytotoxic T cells. The receptor 4-1BB is absent from resting T lymphocytes but is rapidly expressed upon antigenic stimulation. The ligand 4-1BBL can reactivate anergic T lymphocytes and promote T lymphocyte proliferation. 4-1BBL also appears to be required for optimal CD8 responses in CD8 T cells.

721.221 cells: Also referred to as LCL 721.221 or ATCC® CRL-1855™ cells, 721.221 cells are B lymphocytes derived from a human Epstein-Barr virus-transformed cell line. 721.221 cells do not express class I histocompatibility antigens (also known as major histocompatibility complex (MHC) class I molecules), or express low levels of MHC I molecules. Methods of producing 721.221 cells are known in the art (See, e.g., Shimiz et al., Proc Natl Acad Sci USA. 85(1):227-31, 1988, incorporated by reference in its entirety). A population of 721.221 cells may include (such as through transduction or transfection) one or more heterologous nucleic acids, such as a heterologous nucleic acid encoding membrane-bound interleukin 21 (mIL-21). A 721.221 cell that expresses mIL-21 is a mIL-21 positive 721.221 cell. In some examples, a mIL-21 positive 721.221 cell (mIL-21 721.221 cell) includes a construct with the nucleic acid sequence of SEQ ID NO: 3. In some examples, a mIL-21 721.221 cell includes a construct with a nucleic acid sequence encoding the amino acid sequence of SEQ ID NO: 10. Such cells may be identified and/or isolated using various methods known in the art, such as, but not limited to, fluorescence activated cell sorting (FACS).

About: Unless context indicates otherwise, “about” refers to plus or minus 5% of a reference value. For example, “about” 100 refers to 95 to 105.

Administer, Administering, Administration: As used herein, administering a therapeutic agent (e.g., an NK cell, CAR-NK cell, T cell, or CAR-T cell) to a subject means to apply, give, or bring the agent into contact with a subject, by any effective route. Administration can be accomplished by a variety of routes, such as, for example, parenterally, such as intravenous administration. In some examples, a population of cells (such as NK cells, CAR-NK cells, T cells, or CAR-T cells) is administered parenterally, for example intravenously; however, other routes of administration can be utilized. Appropriate routes of administration can be determined based on factors such as the subject, the condition being treated, and other factors.

Autologous: Refers to tissues, cells, or nucleic acids taken from an individual's own tissues. For example, in an autologous transfer or transplantation of cells, the donor and recipient are the same person. Autologous (or “autogeneic” or “autogenous”) is related to self, or originating within an organism itself.

B7-H6: Also referred to as natural killer cell cytotoxicity receptor 3 ligand 1 (NCR3LG1). A B7 family member that is selectively expressed on tumor cells. It binds to NKp30 on NK cells and results in NK cell activation. B7-H6 sequences are publicly available and include GenBank Accession Nos. NM_001202439.3 (human nucleic acid) and NP_001189368.1 (human amino acid). One of ordinary skill in the art can identify additional B7-H6 sequences, for example using public databases.

Cancer: Also referred to as a “malignant tumor” or “malignant neoplasm,” cancer refers to any of a number of diseases characterized by uncontrolled, abnormal proliferation of cells. Cancer cells have the potential to spread locally or through the bloodstream and lymphatic system to other parts of the body (e.g., metastasize) with any of a number of characteristic structural and/or molecular features. A “cancer cell” is a cell having specific structural properties, lacking differentiation, and being capable of invasion and metastasis. Indolent and high-grade forms are included.

Cluster of differentiation 28 (CD28): A protein expressed on T cells that provides co-stimulatory signals important for T cell proliferation and survival, cytokine production, and T helper type-2 development. T cell stimulation through CD28 in addition to the T-cell receptor (TCR) can provide a potent signal for the production of various interleukins (e.g., IL-6). Further, CD28 enhances the production of IL-4 and IL-10 in T cells in conjunction with TCR/CD3 ligation and CD40L co-stimulation.

Centrifugation: A method of separating components (e.g., molecules (such as proteins), cells, vesicles, and similar) of a mixture (such as a supernatant of a cell culture) that have different densities by spinning them in solution around an axis (in a centrifuge rotor) at high speed. Ultracentrifugation is a specialized centrifugation technique used to spin samples at exceptionally high speeds. An ultracentrifuge can be capable of generating accelerations as high as 1,000,000×g (approximately 9,800 km/s²). Serial ultracentrifugation (also known as differential ultracentrifugation) is used to selectively sediment different components within a sample, such as a cell culture supernatant. Samples are centrifuged in successive rounds with increasing centrifugation forces and durations to remove cells, cellular debris, and/or macromolecular proteins, followed by ultracentrifugation (e.g., at 160,000×g for 50-80 minutes) to obtain the desired component (such as exosomes) in the supernatant. In some examples, serial ultracentrifugation is used to separate exosomes from a portion of, substantially all, or all other components of a cell culture supernatant, such as a supernatant from a culture of 721.221-mIL-21 cells.

Chimeric antigen receptor (CAR): A chimeric fusion protein having an extracellular domain that is fused via a transmembrane domain to an intracellular signaling domain capable of activating a T cell or NK cell. In some examples, CAR molecules can include an extracellular domain (ectodomain) with two (or more) targeting domains that are functionally different from each other (multispecific CAR) and that bind to two different sites on a target (multi-targeted). For example, one targeting domain of a multispecific CAR can be a cell surface receptor, such as CD19 (e.g., a multispecific CD19-based CAR). In another example, one targeting domain of a multispecific CAR can be a cell surface receptor, such as CD19, and the second targeting domain can be an antibody or a fragment thereof, such as a scFv (i.e., a multispecific CD19-scFv CAR). In some embodiments, the CD19-scFv CAR binds two different target sites (i.e., a multi-targeted CD19-scFv). A monofunctional CAR contains only a single functional element in the targeting extracellular domain. In some particular embodiments, a portion of the CAR's extracellular binding domain is derived from a murine or humanized monoclonal antibody.

The intracellular signaling domain of CAR molecules include two or more different cytoplasmic signaling domains. For example, one signaling domain can be a cytoplasmic effector function signaling domain and the second signaling domain can be a cytoplasmic co-stimulatory signaling domain. Linkers can connect domains to each other (for example, the two targeting domains) or they can connect one domain to another domain (for example, the ligand-binding domain to the transmembrane domain). CARs are also known as chimeric immune receptors, zetakines, and universal T cell receptors.

Methods of making CARs are available (see, e.g., Park et al., Trends Biotechnol., 29:550-557, 2011; Grupp et al., N Engl J Med., 368:1509-1518, 2013; Han et al., J. Hematol Oncol., 6:47, 2013; PCT Pubs. WO 2012/079000, WO 2013/059593; and U.S. Pub. 2012/0213783, each of which is incorporated by reference herein in its entirety.)

Contacting: Placement in direct physical association, including both a solid and liquid form. In one example, contacting includes association between a substance (such as a cytokine or exosome) or cell in a liquid medium and one or more other cells (such as NK cells or T cells in culture). Contacting can occur in vitro with isolated cells or tissue or in vivo by administering to a subject.

Culturing, Cell culture: Growth of a population of cells in a defined set of conditions (such as culture medium, extracellular matrix, temperature, and/or time of culture) in vitro. In some examples, a cell culture includes a substantially pure culture (for example, isolated 721.221 cells (e.g., mIL-21 721.221 cells or 721.221-mIL21-B7H6 cell) or isolated NK cells). In additional examples a cell culture includes a mixed culture, such as co-culture of two or more types of cells (for example a culture of NK cells with feeder cells). In further examples, a cell culture includes cells grown in contact with an extracellular matrix.

A culture medium is a synthetic set of culture conditions with the nutrients necessary to support the viability, function, and/or growth of a specific population of cells, such as 721.221 cells (e.g., mIL-21 721.221 cells or 721.221-mIL21-B7H6 cells). A culture medium may be a liquid, a solid, or a semi-solid (such as a gel). Culture media generally include components such as a carbon source, a nitrogen source, and a buffer to maintain pH. Additional components in culture media also may include one or more of serum, cytokines, hormones, growth factors, protease inhibitors, protein hydrolysates, shear force protectors, proteins, vitamins, glutamine, trace elements, inorganic salts, minerals, lipids, and/or attachment factors.

As used herein, a supernatant of a cell culture is the culture media (such as a liquid culture medium) in which the cells are cultured. A supernatant may be separated from some, substantially all, or all cells present in a cell culture by any of various methods known to one of ordinary skill in the art, such as, but not limited to, centrifugation (such as ultracentrifugation, such as serial ultracentrifugation), precipitation, crystallization, or settling.

Cytokine: Proteins made by cells that affect the behavior of other cells, such as lymphocytes. In one embodiment, a cytokine is an interleukin, a molecule that regulates cell growth, differentiation, and motility (e.g., to stimulate immune responses, such as inflammation). In other embodiments, the cytokine can be an activating receptor ligand, TLR ligand, or receptors thereof. A cytokine may be heterologous with regard to a cell in which it is expressed. In some examples, the cytokine includes molecules known to stimulate or co-stimulate cell expansion (e.g., NK or T cell expansion). The term “cytokine” is used as a generic name for a diverse group of soluble proteins and peptides that act as humoral regulators at nanomolar to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to, tumor necrosis factor α (TNF-α), interleukin (IL)-2, IL-7, IL-15, IL-21 (including membrane-bound IL-21 (mIL-21)), interferon (IFN)γ, IFNα, IFNβ, IL-12, IL-33, IL-27, IL-18, IL-1 family molecules (e.g., IL-1α, IL-1β, IL-1Rα, IL-18, IL-36Rα, IL36α, IL36β, IL-36γ, IL-37, IL-38, IL-33, toll-like receptor (TLR) ligands, activating receptor ligands (e.g., UL16 binding protein (ULBP)-1, ULPB-2, major histocompatibility complex (MHC) class I chain-related protein A (MIC-A)), IL-1 family molecules, Fc receptors, intercellular adhesion molecule 1 (ICAM-1), CD8a, 2B4 (also known as cluster of differentiation 244 (CD244)), intercellular adhesion molecule 1 (ICAM-1), CD8a, CD40, CD28, 4-1BBL, OX40L, TRX518, CD3 antibody, and CD28 antibody.

Effective amount: A quantity of a specified agent sufficient to achieve a desired effect. In some examples, an effective amount of exosomes isolated or purified from a population of 721.221-mIL-21 cells or 721.221-mIL21-B7H6 cells is an amount sufficient to promote expansion of a population of immune cells (such as an NK cell or T cell). In other examples, an effective amount of an expanded NK cell or T cell (e.g., a CAR-NK cell or CAR-T cell) disclosed herein is an amount sufficient to treat or inhibit a disease or disorder in a subject (such as a tumor, viral infection, autoimmune disease, or transplant rejection). In other examples, an effective amount is an amount of an expanded NK cell or T cell (e.g., a CAR-NK cell or CAR-T cell) sufficient to reduce or ameliorate one or more symptoms of a disease or disorder in a subject. The effective amount (for example, an amount ameliorating, inhibiting, and/or treating a disorder in a subject) will be dependent on, for example, the particular disorder being treated, the subject being treated, the manner of administration of the composition, and other factors.

Exosome: Exosomes are a class of cell-derived extracellular vesicles of endosomal origin and can be 30-150 nm in diameter. Enveloped by a lipid bilayer, exosomes are released into the extracellular environment and contain components derived from the original cell, such as, but not limited to, proteins, lipids, RNA (such as mRNA and/or miRNA), and/or DNA. Exosomes are formed through the fusion and exocytosis of multivesicular bodies into the extracellular space. Multivesicular bodies are organelles in the endocytic pathway that function as intermediates between early and late endosomes. A function of multivesicular bodies is to separate components that will be recycled elsewhere from those that will be degraded by lysosomes. The vesicles that accumulate within multivesicular bodies are categorized as intraluminal vesicles while inside the cytoplasm and exosomes when released from the cell. Intraluminal vesicles are thus essentially exosome precursors, and form by budding into the lumen of the multivesicular body. Intraluminal vesicles may fuse with lysosomes for subsequent degradation or may be released into the extracellular space. The intraluminal vesicles that are secreted into the extracellular space when the multivesicular body fuses with the plasma membrane are termed exosomes. In some examples, the exosomes are derived from 721.221 cells, such as 721.221-mIL-21 cells or 721.221-mIL21-B7H6 cells, and can be isolated from a supernatant of a cell culture of a population of such cells using methods described herein.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

Feeder cells (FCs): Cells that provide support for another cell type in ex vivo or in vitro culture. Feeder cells may provide one or more factors required for survival, growth, and/or differentiation (or inhibiting differentiation) of the cells cultured with the feeder cells. Typically, feeder cells are irradiated or otherwise treated to prevent their proliferation in culture. In some examples disclosed herein, immune cells (such as NK cells) are cultured with feeder cells, such as irradiated modified 721.221 cells (e.g., mIL-21-expressing 721.221 cells or mIL-21 and B7-H6 expressing 721.221 cells). In other examples disclosed herein, immune cells (such as NK cells) are cultured in the absence of feeder cells.

Immunoglobulin 1 (IgG1): The IgG1 Fc is a dimeric protein that mediates important antibody effector functions by interacting with Fcγ receptors (FcγRs) and the neonatal Fc receptor (FcRn). The IgG Fc region comprises two CH2 and two CH3 domains. The IgG1 CH3 domain has two important functions: dimerization of the IgG1 Fc and interaction with the neonatal Fc receptor (FcRn). The IgG1 Fc can interact with the neonatal Fc receptor (FcRn), which salvages the antibody from lysosomal degradation and thereby extends its in vivo half-life. The Fc can also bind to multiple Fc receptors to induce effector functions, including antibody-dependent cell-mediated cytotoxicity (ADCC) and complement-dependent cytotoxicity (CDC).

Immune disease: A disorder or disease, such as an autoimmune disorder or disease, in which the immune system produces an immune response (e.g., a B cell or a T cell response) against an endogenous antigen, with consequent injury to tissues. The injury may be localized to certain organs, such as thyroiditis, or may involve a particular tissue at different locations, such as Goodpasture's disease, or may be systemic, such as lupus erythematosus.

In some examples, autoimmune diseases include systemic lupus erythematosus, Sjogren's syndrome, rheumatoid arthritis, type I diabetes mellitus, Wegener's granulomatosis, inflammatory bowel disease, polymyositis, dermatomyositis, multiple endocrine failure, Schmidt's syndrome, autoimmune uveitis, Addison's disease, adrenalitis, Graves' disease, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, multiple sclerosis, subacute cutaneous lupus erythematosus, hypoparathyroidism, Dressler's syndrome, myasthenia gravis, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), adult onset diabetes mellitus (Type II diabetes), male and female autoimmune infertility, ankylosing spondylitis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, glomerulonephritis, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and Non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, rubulavirus, and Evan's syndrome.

Infectious disease: Also known as transmissible disease or communicable disease, infectious diseases are illnesses resulting from an infection. Infections are caused by infectious agents, including viruses, viroids, prions, bacteria; nematodes, such as parasitic roundworms and pinworms; arthropods, such as ticks, mites, fleas, and lice; fungi, such as ringworm; and other macroparasites, such as tapeworms and other helminths. Hosts fight infections using the immune system, such as the innate response (e.g., in mammals), which involves inflammation, followed by an adaptive response. Medications used to treat infections include antibiotics, antivirals, antifungals, antiprotozoals, and antihelminthics. Specific, non-limiting examples of infectious diseases include human immunodeficiency syndrome (HIV), human papillomavirus (HPV), hepatitis B virus (HBV), hepatitis C virus (HVC), tuberculosis (TB), and malaria.

Interleukin: Interleukins (ILs) are a type of cytokine involved in the activation and differentiation of immune cells, as well as cell proliferation, maturation, migration, and adhesion. Interleukins modulate immune cell growth, differentiation, and activation during inflammatory and immune responses. Interleukins broadly include a group of proteins that each include a four alpha helix bundle, and can elicit reactions in cells and tissues by binding to high-affinity receptors on cell surfaces. Interleukins have both paracrine and autocrine functions. Exemplary interleukins are described herein.

Interleukin 2 (IL-2): IL-2 is a cytokine that principally targets T cells, and T cells (primarily activated CD4+ T cells and activated CD8+ T cells) also produce IL-2. IL-2 signals through the IL-2 receptor, which includes three chains: alpha (CD25), beta (CD122) and gamma (CD132). The IL-2 receptor (IL-2R) a subunit binds IL-2 with low affinity (Kd˜10-8 M). Interaction of IL-2 and CD25 alone does not lead to signal transduction due to the short CD25 intracellular chain but can (when bound to the β and γ subunits) increase IL-2R affinity 100-fold. Heterodimerization of the IL-2R β and γ subunits is essential for IL-2 signaling in T cells. IL-2 can signal either through intermediate-affinity dimeric CD122/CD132 IL-2R (Kd˜10-9 M) or high-affinity trimeric CD25/CD122/CD132 IL-2R (Kd˜10-11 M). Dimeric IL-2R is expressed by memory CD8+ T cells and NK cells, whereas regulatory T cells and activated T cells express high levels of trimeric IL-2R. IL-2 signaling results in T-cell proliferation and differentiation, increased cytokine synthesis, potentiating Fas-mediated apoptosis, and promoting regulatory T cell development. IL-2 also induces proliferation and activation of NK cells and proliferation and antibody synthesis in B cells, and stimulates activation of cytotoxic lymphocytes and macrophages. Exemplary nucleotide and amino acid sequences encoding IL-2 are provided in SEQ ID NOs: 8 and 9, respectively.

Interleukin 15 (IL-15): IL-15 is a cytokine with structural similarity to IL-2, and is constitutively expressed by a large number of cell types and tissues, including monocytes, macrophages, dendritic cells, keratinocytes, fibroblasts, myocyte and nerve cells. Exemplary nucleotide and amino acid sequences encoding IL-15 are provided in SEQ ID NOs: 6 and 7, respectively. IL-15 binds to and signals through a complex composed of IL-2/IL-15 receptor P chain (CD122), the common γ chain (γ-C, CD132), and IL-15Rα. Exemplary nucleotide and amino acid sequences encoding IL-15Rα are provided in SEQ ID NOs: 4 and 5, respectively. IL-15Rα specifically binds IL-15 with very high affinity (and can bind IL-2 with low affinity) and is capable of binding IL-15 independently of other subunits. IL-15 is secreted by mononuclear phagocytes (and some other cells) following infection by virus(es). This cytokine regulates activation and proliferation of NK cells and T cells.

Interleukin 21 (IL-21): IL-21 is a pleiotropic cytokine with actions on a broad range of lymphoid, myeloid, and epithelial cells. These actions include effects on proliferation, survival, differentiation, and function. IL-21 is expressed in activated CD4+ T cells. IL-21 expression is up-regulated in Th2 and Th17 subsets of T helper cells, as well as T follicular cells. The IL-21 receptor (IL-21R) is expressed on the surface of T cells, B cells, and NK cells, and IL-21 signaling induces B cell and T cell activation, and enhances NK cell activity. IL-21R is similar in structure to the receptors for other type I cytokines like IL-2 or IL-15 and requires dimerization with the common gamma chain (y-c) in order to bind IL-21. In some examples, IL-21 is membrane bound (mIL-21), such as in 721.221-mIL-21 cells described herein. Exemplary nucleotide and amino acid sequences encoding the IL-21 extracellular domain are provided in SEQ ID NO: 1 and SEQ ID NO: 2, respectively.

Isolated: An “isolated” or “purified” biological component (such as a cell, nucleic acid, peptide, protein, protein complex, or exosome) has been substantially separated, produced apart from, or purified away from other components (for example, other biological components in the cell or environment in which the component naturally occurs). Cells, nucleic acids, peptides and proteins, or exosomes that have been “isolated” or “purified” thus include cells, nucleic acids, proteins, or exosomes purified by standard purification methods.

The term “isolated” or “purified” does not require absolute purity; rather, it is intended as a relative term. Thus, for example, an isolated biological component is one in which the biological component is more enriched than the biological component is in its natural environment within a cell, organism, sample, or production vessel (for example, a cell culture system). Preferably, a preparation is purified such that the biological component represents at least 50%, such as at least 70%, at least 80%, at least 90%, at least 95%, or greater, of the total biological component content of the preparation.

Killer-cell immunoglobulin-like receptor (KIR), KIR ligand: KIRs are a family of type I transmembrane glycoproteins expressed on the plasma membrane of NK cells and a minority of T cells. KIRs regulate the killing functions of these cells by interacting with major histocompatibility (MHC) class I molecules (such as HLA-C2, HLA-C1, HLA-Bw4, HLA-G, HLA-A, or HLA-F), which are expressed on all nucleated cell types. KIR receptors can distinguish between MHC class I allelic variants, which allows them to detect virally infected cells or transformed cells. Recognition of MHC molecules by inhibitory KIRs suppresses the cytotoxic activity of the NK cell. Only a limited number of KIRs are activating, meaning that their recognition of MHC molecules activates the cytotoxic activity of their cell.

Macrophage: A type of white blood cell that phagocytoses and degrades cellular debris, foreign substances, microbes, and cancer cells. Macrophages also play an important role in development, tissue maintenance and repair, and in both innate and adaptive immunity in that they recruit and influence other cells including immune cells such as lymphocytes. Macrophages can exist in many phenotypes, including phenotypes that have been referred to as M1 and M2. Macrophages that perform primarily pro-inflammatory functions are called M1 macrophages (CD86+/CD68+), whereas macrophages that decrease inflammation and encourage and regulate tissue repair are called M2 macrophages (CD206+/CD68+). The markers that identify the various phenotypes of macrophages vary among species. The degree to which a given macrophage bears M1 or M2 characteristics is termed “polarization” (Taylor et al., Annu Rev Immunol. 23:901-944, 2005). Macrophage polarization is a process by which macrophages adopt different functional programs in response to the signals from their microenvironment. Markers are used to determine the polarization status and alteration of function.

Purified macrophages include macrophage cells enriched (such as using the enrichment methods disclosed herein) from populations of mononuclear cells, such as peripheral blood mononuclear cells (PBMCs). An expanded macrophage refers to a macrophage derived from a primary macrophage or monocyte by ex vivo or in vitro cell culture. In some examples, expanded macrophages are macrophages that have been differentiated from primary monocytes (such as primary monocytes from a population of PBMCs) in the presence of one or more cytokines (such as macrophage colony-stimulating factor (M-CSF), granulocyte-macrophage colony-stimulating factor (GM-CSF), IL-4, IL-10, IFN-γ, and/or TGF-β), and then contacted with the disclosed exosomes for a period of time in order to increase the number of macrophage cells. Macrophages may be differentiated from monocytes isolated from PBMCs, such as from cells of peripheral blood collected from a subject. In some examples, expanded macrophages can exhibit altered gene expression compared to primary (non-expanded) macrophages. Macrophages can be expanded directly from monocytes present in a mixed population of cells (such as PBMCs), using the exosomes.

In some examples, a modified macrophage is a macrophage with increased and/or decreased expression of one or more genes compared to an unmodified macrophage. In some examples, a modified macrophage is transduced with a heterologous nucleic acid or expresses one or more heterologous proteins. In other examples, a modified macrophage has a modification that decreases expression of one or more genes. The terms “modified macrophage” and “transduced macrophage” are used interchangeably in some examples herein. In some examples, a CAR-macrophage or CAR-M cell is a macrophage transduced with a heterologous nucleic acid encoding or expressing a CAR.

Major histocompatibility complex class I (MHC class I): A class of MHC molecules typically found on the cell surface of all nucleated cells in the bodies of vertebrates. They also occur on platelets, but not on red blood cells. MHC class I complexes display peptide fragments of proteins from within the cell (e.g., peptides derived from cytosolic proteins) to cytotoxic T cells. MHC class I molecules are heterodimers that consist of two polypeptide chains, α and β2-microglobulin (B2M). The two chains are linked noncovalently via interaction of B2M and the α3 domain. The α3 domain is plasma membrane-spanning and interacts with the CD8 co-receptor of T cells. The α3-CD8 interaction holds the MHC I molecule in place while the T cell receptor (TCR) on the surface of the cytotoxic T cell binds its α1-α2 heterodimer ligand and checks the coupled peptide for antigenicity. The α1 and α2 domains fold to make up a groove for peptides to bind. The T cell interaction triggers an immune response against the particular antigen displayed. In humans, the MHC class II protein complex is encoded by the HLA gene complex. HLAs corresponding to MHC class I include HLA-A, HLA-B, and HLA-C.

Major histocompatibility complex class II (MHC class II): A class of MHC molecules typically found on professional antigen-presenting cells, such as dendritic cells, mononuclear phagocytes, some endothelial cells, thymic epithelial cells, and B cells. These cells are important in initiating immune responses. The antigens presented by MHC class II molecules are derived from extracellular proteins (not cytosolic proteins as in MHC class I). MHC class II molecules are formed from two noncovalently associated proteins, the α chain and the β chain. The a chain comprises α1 and α2 domains, and the β chain comprises β1 and β2 domains. The cleft into which the antigen fits is formed by the interaction of the al and β1 domains. The α2 and β2 domains are transmembrane Ig-fold like domains that anchor the α and β chains into the cell membrane of the APC. MHC class II complexes, when associated with antigen (and in the presence of appropriate co-stimulatory signals) stimulate CD4 T cells to initiate inflammatory responses, regulate other cells in the immune system, and provide assistance to B cells for antibody synthesis. HLAs corresponding to MHC class II include HLA-DP, HLA-DM, HLA-DOA, HLA-DOB, HLA-DQ, and HLA-DR.

Natural Killer (NK) cells: Cells of the immune system that kill target cells in the absence of a specific antigenic stimulus and without restriction according to MHC class. Target cells can be tumor cells or cells harboring viruses. NK cells are characterized by the presence of CD56 and the absence of CD3 surface markers. NK cells typically comprise approximately 10 to 15% of the mononuclear cell fraction in normal peripheral blood. Historically, NK cells were first identified by their ability to lyse certain tumor cells without prior immunization or activation. NK cells are thought to provide a “back up” protective mechanism against viruses and tumors that might escape the cytotoxic T lymphocyte (CTL) response by down-regulating MHC class I presentation. In addition to being involved in direct cytotoxic killing, NK cells also serve a role in cytokine production, which can be important to control cancer and infection. Tissue-resident memory NK cells are included.

Purified NK cells (pNKs) include NK cells enriched (such as using the enrichment methods disclosed herein) from populations of mononuclear cells, such as peripheral blood mononuclear cells (PBMCs). An expanded NK cell refers to an NK cell that has been derived from a primary NK cell by ex vivo or in vitro cell culture in order to increase the number of NK cells. NK cells may be expanded from PBMCs or from pNK cells, such as from cells of peripheral blood collected from a subject. In some examples, expanded NK cells can exhibit altered gene expression compared to primary (non-expanded) NK cells.

In some examples, a modified NK cell is an NK cell with increased and/or decreased expression of one or more genes compared to an unmodified NK cell. In some examples, a modified NK cell is transduced with a heterologous nucleic acid or expresses one or more heterologous proteins. In other examples, a modified NK cell has a modification that decreases expression of one or more genes. The terms “modified NK cell” and “transduced NK cell” are used interchangeably in some examples herein. In some examples, a CAR-NK cell is an NK cell transduced with a heterologous nucleic acid encoding or expressing a CAR. In some examples, the CAR-NK cell is a CD19 CAR-NK cell or a CD147 CAR-NK cell.

Natural killer T (NKT) cell: A heterogeneous group of T cells that share properties of both T cells and NK cells. NKT cells co-express an αβ T cell receptor and a variety of molecular markers that are typically associated with NK cells, such as NK1.1. NKT cells constitute only approximately 1% of all peripheral blood T cells. Many of these cells recognize the non-polymorphic CD1d molecule, an antigen-presenting molecule that binds self and foreign lipids and glycolipids. Upon activation, NKT cells can produce large quantities of interferon gamma, IL-4, and granulocyte-macrophage colony-stimulating factor (GM-CSF), as well as multiple other cytokines and chemokines (such as IL-2, IL-13, IL-17, IL-21, IL-13, IL-6, IFN-γ, and/or TNF-α).

Purified NKT cells (pNKTs) include NKT cells enriched (such as using the enrichment methods disclosed herein) from populations of mononuclear cells, such as peripheral blood mononuclear cells (PBMCs). An expanded NKT cell refers to an NKT cell that has been derived from a primary NKT cell by ex vivo or in vitro cell culture for a period of time in order to increase the number of NKT cells. NKT cells may be expanded from PBMCs cells, such as from cells of peripheral blood collected from a subject. In some examples, expanded NKT cells can exhibit altered gene expression compared to primary (non-expanded) NKT cells.

In some examples, a modified NKT cell is an NKT cell with increased and/or decreased expression of one or more genes compared to an unmodified NKT cell. In some examples, a modified NKT cell is transduced with a heterologous nucleic acid or expresses one or more heterologous proteins. In other examples, a modified NKT cell has a modification that decreases expression of one or more genes. The terms “modified NKT cell” and “transduced NKT cell” are used interchangeably in some examples herein. In some examples, a CAR-NKT cell is an NKT cell transduced with a heterologous nucleic acid encoding or expressing a CAR (Nelson et al., Cancers, 13(20):5147, 2021).

Peripheral blood mononuclear cell (PBMC): Peripheral blood mononuclear cells (PBMCs) include any blood cell with a round nucleus (e.g., lymphocytes (NK cells, T cells, and B cells), monocytes, macrophages, or dendritic cells). In humans, the frequencies of these populations vary across individuals, but typically, lymphocytes are in the range of 70-90%, monocytes from 10-20%, while dendritic cells are rare, accounting for only 1-2%. The frequencies of cell types within the lymphocyte population include about 70-85% CD3+ T cells, about 5-10% B cells, and about 5-20% NK cells.

During PBMC collection from a subject, the cell fraction corresponding to red blood cells and granulocytes (neutrophils, basophils, and eosinophils) is removed from whole blood by density gradient centrifugation. A gradient medium with a density of 1.077 g/ml separates whole blood into two fractions. PBMCs make up the population of cells that remain in the low density fraction (upper fraction), while red blood cells and polymorphonuclear leukocytes (PMNs) have a higher density and are found in the lower fraction.

Pharmaceutically acceptable carrier: Remington: The Science and Practice of Pharmacy, The University of the Sciences in Philadelphia, Editor, Lippincott, Williams, & Wilkins, Philadelphia, PA, 21^{st} Edition (2005) describes compositions and formulations suitable for pharmaceutical delivery of one or more therapeutic compositions, such as one or more immune cells and/or additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Purified: The term purified does not require absolute purity; rather, it is intended as a relative term. Thus, for example, a purified protein or cell preparation is one in which the protein or cell is more enriched than in its original environment. In one embodiment, a preparation is purified such that the protein or cells (such as purified exosomes or NK cells) represent at least 50% of the total content of the preparation.

Subject: A living multi-cellular vertebrate organism, a category that includes both human and non-human mammals (such as veterinary or laboratory animals, including dogs and cats, as well as mice, rats, rabbits, sheep, horses, cows, and non-human primates).

T Cell: A white blood cell (lymphocyte) that is an important mediator of the immune response. T cells include, but are not limited to, CD4+ T cells and CD8+ T cells. A CD4+ T lymphocyte is an immune cell that expresses CD4 on its surface. These cells, also known as helper T cells, help orchestrate the immune response, including antibody responses as well as killer T cell responses. Th1 and Th2 cells are functional subsets of helper T cells. Th1 cells secrete a set of cytokines, including interferon-gamma, and whose principal function is to stimulate phagocyte-mediated defense against infections, especially related to intracellular microbes. Th2 cells secrete a set of cytokines, including interleukin (IL)-4 and IL-5, and whose principal functions are to stimulate IgE and eosinophil/mast cell-mediated immune reactions and to downregulate Th1 responses. In further examples, T cells can include regulatory T cells (Tregs), NKT cells, tumor infiltrating lymphocytes (TIL), other unconventional T cells (e.g., MAIT, γδ T cells, and CD8αα+ IELs), innate lymphoid cells (ILCs), tissue-resident memory T cells, or any vaccine-primed T cells. Similar to CD4+ T cells, Tregs also express CD4 but are distinguished by expression of TGFβ. Tregs can aid in treating immune disorders, such as autoimmune disease, chronic graft versus host disease (GVHD), diabetes, systemic lupus erythematosus, obesity, and encephalitis, as well as facilitate organ transplant acceptance.

An expanded T cell refers to a T cell that has been derived from a primary T cell by ex vivo or in vitro cell culture for a period of time in order to increase the number of T cells. In some examples, expanded T cells can exhibit altered gene expression compared to primary (non-expanded) T cells.

In some examples, a modified T cell is a T cell with increased and/or decreased expression of one or more genes compared to an unmodified T cell. In some examples, a modified T cell is transduced with a heterologous nucleic acid or expresses one or more heterologous proteins. In other examples, a modified T cell has a modification that decreases expression of one or more genes. The terms “modified T cell” and “transduced T cell” are used interchangeably in some examples herein. In some examples, a “CAR-T cell” is a T cell transduced with a heterologous nucleic acid encoding or expressing a CAR.

T cell surface glycoprotein CD3 zeta chain (CD3ζ): Part of the TCR-CD3 complex present on T-lymphocyte cell surface that plays an essential role in adaptive immune response. Also known as CD247. CD3 zeta, together with T cell receptor alpha/beta and gamma/delta heterodimers and CD3-gamma, -delta, and -epsilon, forms the T-cell receptor-CD3 complex. When antigen presenting cells activate TCR, TCR-mediated signals are transmitted across the cell membrane by CD3 delta, CD3 epsilon, CD3 gamma, and CD3 zeta. All CD3 chains contain immunoreceptor tyrosine-based activation motifs (ITAMs) in their cytoplasmic domain. Upon TCR engagement, these motifs become phosphorylated by Src family protein tyrosine kinases LCK and FYN, resulting in the activation of downstream signaling pathways. Thus, the zeta chain plays a role in coupling antigen recognition to several intracellular signal-transduction pathways.

Toll-like receptor (TLR), TLR ligand: Toll-like receptors (TLRs) are a class of pattern recognition receptors (PRRs) that initiate the innate immune response by sensing conserved molecular patterns for early immune recognition of a pathogen. TLRs are expressed in innate immune cells such as dendritic cells and macrophages as well as non-immune cells such as fibroblast cells and epithelial cells (Kawasaki et al., Front. Immunol, 5:1-8, 2014). Numerous roles for TLRs have been identified, such as recognition of self and non-self antigens; detection of invading pathogens; bridging the innate and adaptive immunity responses; and regulation of cytokine production, proliferation, and survival. Generally, TLRs are type I transmembrane proteins that contain three structural domains: a leucine-rich repeats (LRR) motif, a transmembrane domain, and a cytoplasmic Toll/IL-1 receptor (TIR) domain (Nie et al., Front. Immunol. 9:1-19, 2018). The LRR motif is responsible for pathogen recognition, whereas the TIR domain interacts with signal transduction adaptors and initiates signaling.

TLRs can recognize molecules (“TLR ligands”) broadly shared by pathogens, known as pathogen-associated molecular patterns (PAMPs), and host endogenous damage-associated molecular pattern molecules (DAMPs). These TLR ligands are often TLR agonists that activate TLR signaling and are evolutionarily conserved. TLR agonists include pathogen-associated molecules, such as bacterial cell-surface lipopolysaccharides (LPS), lipoproteins, lipopeptides, and lipoarabinomannan; proteins, such as flagellin from bacterial flagella; double-stranded RNA of viruses; unmethylated CpG islands of bacterial and viral DNA; CpG islands in the eukaryotic DNA promoters; as well as other RNA and DNA molecules. Additional exemplary TLR ligands include CpG-oligodeoxynucleotides, resiquimod (R848), IL-2, phytohemagglutinin (PHA), 4β,9α,12β,13α,20-Pentahydroxytiglia-1,6-dien-3-one 12-tetradecanoate 13-acetate (phorbol 12-myristate 13-acetate, PMA), ionomycin, and polyinosinic-polycytidylic acid (poly(I:C)).

TLR-ligand recognition is multifarious, depending on the type of TLR. TLRs are largely classified into two subfamilies based on their localization, cell surface TLRs and intracellular TLRs. Cell surface TLRs include TLR1, TLR2, TLR4, TLR5, TLR6, and TLR10, whereas intracellular TLRs are localized in the endosome and include TLR3, TLR7, TLR8, TLR9, TLR11, TLR12, and TLR13. Cell surface TLRs mainly recognize microbial membrane components such as lipids, lipoproteins, and proteins. Intracellular TLRs recognize nucleic acids derived from bacteria and viruses, and also recognize self-nucleic acids in disease conditions such as autoimmunity. TLR functions are mediated by subsequently initiated signaling pathways, resulting in the production of various cytokines and chemokines. TLR activation generally results in activation and phenotypic maturation of dendritic cells.

Transformed: A transformed cell is a cell into which has been introduced a nucleic acid molecule by molecular biology techniques. As used herein, the term transformation encompasses all techniques by which a nucleic acid molecule might be introduced into such a cell, including transduction with viral vectors, transfection with plasmid vectors, and introduction of naked DNA by electroporation, lipofection, and particle gun acceleration (e.g., ‘transfection’).

Transgene: A heterologous nucleic acid introduced into a cell, for example, by transduction. In some examples, a transgene is a nucleic acid encoding a protein of interest. In other examples, a transgene includes a nucleic acid that is capable of modulating expression of a nucleic acid of interest, such as a sgRNA, small interfering RNA (siRNA), or antisense nucleic acid. The transgene may be operably linked to one or more expression control sequences, for example, a promoter.

A “heterologous” nucleic acid or protein refers to a nucleic acid or protein originating from a different genetic source. For example, a nucleic acid or protein that is heterologous to a cell originates from an organism or individual or cell type other than the cell in which it is expressed (for example, a nucleic acid or protein not normally present in NK cells is heterologous to NK cells). In further examples, a heterologous nucleic acid includes a recombinant nucleic acid, such as a protein-encoding nucleic acid operably linked to a promoter from another gene and/or two or more operably linked nucleic acids from different sources. Methods for introducing a heterologous nucleic acid molecule into a cell or organism are well known in the art, for example transformation with a nucleic acid, including electroporation, lipofection, particle gun acceleration, and homologous recombination, or transduction of the nucleic acid using a viral vector.

Treating or inhibiting a disorder: “Inhibiting” a disease or disorder refers to inhibiting the full development of a disease or disorder, for example, a cancer (e.g., a tumor or hematological malignancy). Inhibition of a disease or disorder can span the spectrum from partial inhibition to substantially complete inhibition of a disease or disorder (such as a cancer, infection, or immune disease, such as a transplant rejection). In some examples, the term “inhibiting” refers to reducing or delaying the onset or progression of a disease or disorder. “Treatment” refers to a therapeutic intervention that ameliorates a sign or symptom of a disease or disorder after it has begun to develop. The term “ameliorating,” with reference to a disease or disorder, refers to any observable beneficial effect of the treatment. The beneficial effect can be evidenced, for example, by a delayed onset of clinical symptoms of the disease or disorder in a susceptible subject, a reduction in severity of some or all clinical symptoms of the disease or disorder, a slower progression of the disease or disorder, an improvement in the overall health or well-being of the subject, or by other parameters well known in the art that are specific to the particular disease or disorder, such as improved survival of a subject having a cancer. Treatment may be assessed by objective or subjective parameters, including, but not limited to, the results of a physical examination, imaging, or a blood test. A “prophylactic” treatment is a treatment administered to a subject who does not exhibit signs of a disease or disorder or exhibits only early signs for the purpose of decreasing the risk of developing pathology, such as to inhibit the occurrence or recurrence of a cancer. A subject to be administered an effective amount of the disclosed immune cells (such as NK cells, CAR-NK cells, T cells, CAR-T cells, macrophages, CAR-M cells, NKT cells, or CAR-NKT cells) can be identified by standard diagnosing techniques for such a disease or disorder, for example, presence of the disease or disorder or risk factors to develop the disease or disorder.

Vector: A nucleic acid molecule allowing insertion of foreign or heterologous nucleic acid into a cell. A vector can be a nucleic acid molecule (such as a DNA or RNA molecule) including a promoter(s) that is operably linked to the coding sequence of a protein of interest and can express the coding sequence. A vector can include nucleic acid sequences that permit it to replicate in a host cell, such as an origin of replication. A vector can also include one or more selectable marker genes and other genetic elements. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. An expression vector is a vector that contains the necessary regulatory sequences to allow transcription and/or translation of an inserted gene or genes. In some embodiments, a vector includes a nucleic acid molecule encoding an interleukin, such as IL-21, such as membrane-bound IL-21 (mIL-21), B7-H6, or both. In some examples, a vector encodes metabolism regulatory factors, such as all or a portion of IgG1, T cell surface glycoprotein CD3 zeta chain (CD3ζ), 4-1BB, CD28, or a combination thereof. In some examples, the vector is a bacterial vector. In some examples, the vector is a plasmid. In some examples, the vector is a viral vector, such as a retroviral vector or lentiviral vector. A viral vector is a nucleic acid vector having at least some nucleic acid sequences derived from one or more viruses. In some examples, the retroviral vector is a Moloney murine leukemia virus (MoMLV) vector, such as an SFG retroviral vector.

### II. Isolated Exosomes and Methods of Producing the Isolated Exosomes

Disclosed herein are methods of producing an isolated exosome, for example, for use in methods of expanding a population of immune cells (such as NK cells, CAR-NK cells, T cells, CAR-T cells, macrophages, CAR-M cells, NKT cells, CAR-NKT cells, basophils, CAR-basophils, eosinophils, CAR-eosinophils, monocytes, CAR-monocytes, B cells, CAR-B cells, mast cells, or CAR-mast cells), and in methods of treating a cancer or immune or infectious disease using the expanded population of immune cells. Also disclosed is an isolated exosome produced using the methods described herein.

Isolated exosomes disclosed herein are produced, for example, by isolating exosomes from a culture of a population of 721.221 cells modified to include a nucleic acid encoding mIL-21. In some embodiments, the exosomes are isolated or purified from a supernatant of a culture of 721.221 cells modified to include a heterologous nucleic acid encoding membrane-bound IL-21 (mIL-21) (“721.221-mIL-21” cells). In particular embodiments, the 721.221-mIL-21 cells include the mIL-21 construct included in the nucleic acid sequence of SEQ ID NO: 3. In another particular embodiment, the 721.221-mIL-21 cells include a mIL-21 construct that has a nucleic acid sequence encoding the amino acid sequence of SEQ ID NO: 2. In some embodiments, the 721.221-mIL-21 cells are those described in International Patent Application Publication No. WO 2020/172328, which is incorporated herein by reference in its entirety.

In additional embodiments, provided herein are 721.221-mIL-21 cells that also express B7-H6 (721.221-mIL21-B7H6 cells). In particular examples, the 721.221-mIL21-B7H6 cells include a nucleic acid including at least 90% or 95% sequence identity to SEQ ID NO: 11 or including or consisting of SEQ ID NO: 11. In another particular example, the 721.221-mIL21-B7H6 cells include a nucleic acid sequence encoding an amino acid sequence with at least 90% or 95% sequence identity to SEQ ID NO: 12, or including or consisting of SEQ ID NO: 12. In some embodiments, isolated exosomes are produced from a culture of a population of the 721.221-mIL21-B7H6 cells. Isolated exosomes disclosed herein are produced, for example, by isolating exosomes from a culture of a population of 721.221 cells modified to include a nucleic acid encoding mIL-21 and a nucleic acid encoding B7-H6. In some embodiments, the exosomes are isolated or purified from a supernatant of a culture of 721.221 cells modified to include a heterologous nucleic acid encoding membrane-bound IL-21 (mIL-21) (“721.221-mIL-21” cells) and a heterologous nucleic acid encoding B7-H6.

721.221-mIL-21 or 721.221-mIL21-B7H6 cells useful in the disclosed methods may be further modified to include one or more additional heterologous nucleic acids. In some embodiments, the one or more additional heterologous nucleic acid encodes a protein that facilitates expansion of immune cells (such as NK cells, T cells, macrophages, or NKT cells), such as a cytokine (e.g., IL-21, IL-2, IL-12, IL-33, IL-27, IL-18, IL-7, mIL-7, IL-15, mIL-15, a toll receptor (TLR) ligand, or an activating receptor ligand (e.g., UL16 binding protein (ULBP)-1, ULPB-2, major histocompatibility complex (MHC) class I chain-related protein A (MIC-A)), IL-1 family molecules, Fc receptors, intercellular adhesion molecule 1 (ICAM-1), CD8α, 2B4 (also known as cluster of differentiation 244 (CD244)), intercellular adhesion molecule 1 (ICAM-1), and CD8α), including CD40, CD28, 4-1BB ligand (4-1BBL), OX40L, TRX518, CD3 antibody, and CD28 antibody. In some embodiments, the 721.221-mIL-21 cells further express IL-15 receptor α (IL-15Rα). In further examples, the cytokine or cytokine receptor is membrane-bound (e.g., membrane-bound IL-15). In further, non-limiting examples, the 721.221-mIL-21 cells include heterologous nucleic acids encoding membrane-bound ICAM-1, Fc receptor, CD8α, ULBP-1, ULPB-2, or MIC-A.

In some examples, the nucleic acid encoding mIL-21 includes the extracellular domain from IL-21, which may include or consist of a nucleic acid with at least 90% identity (such as at least 90%, 91%, 92%, 93%, 94%, 95%, 96%, 97%, 98%, 99%, or 100% identity) to SEQ ID NO: 1 and/or encodes a protein including or consisting of an amino acid sequence with at least 95% identity (such as at least 95%, 96%, 97%, 98%, 99%, or 100% identity) to SEQ ID NO: 2. In additional examples, the nucleic acid encoding B7-H6 includes a nucleic acid with at least 90% identity (such as at least 90%, 91%, 92%, 93%, 94%, 95%, 96%, 97%, 98%, 99%, or 100% identity) to SEQ ID NO: 11 and/or encodes a protein including or consisting of an amino acid sequence with at least 95% identity (such as at least 95%, 96%, 97%, 98%, 99%, or 100% identity) to SEQ ID NO: 12. 721.221-mIL-21 cells can be produced by transducing or transfecting 721.221 cells with a heterologous nucleic acid encoding mIL-21, and optionally at least one additional heterologous nucleic acid (such as a nucleic acid encoding one or more of IL-2, IL-12, IL-33, IL-27, IL-18, IL-7, mIL-7, IL-15, mIL-15, a TLR ligand, ULBP-1, ULPB-2, MIC-A, IL-1 family molecules, Fc receptors, 2B4, ICAM-1, CD8α, CD40, CD28, 4-1BB, 4-1BBL, OX40L, TRX518, CD3 antibody, or CD28 antibody, and, in some examples, also IL-15Rα). In other examples, 721.221-mIL21-B7H6 cells can be produced by transducing or transfecting 721.221 cells with a heterologous nucleic acid encoding mIL-21 and a heterologous nucleic acid encoding B7-H6. In further examples, 721.221-mIL21-B7H6 cells can be produced by transducing or transfecting 721.221-mLI21 cells with a heterologous nucleic acid encoding B7-H6. The cell may optionally be transduced or transfected with at least one additional heterologous nucleic acid (such as a nucleic acid encoding one or more of IL-2, IL-12, IL-33, IL-27, IL-18, IL-7, mIL-7, IL-15, mIL-15, a TLR ligand, ULBP-1, ULPB-2, MIC-A, IL-1 family molecules, Fc receptors, 2B4, ICAM-1, CD8α, CD40, CD28, 4-1BB, 4-1BBL, OX40L, TRX518, CD3 antibody, or CD28 antibody, and, in some examples, also IL-15Rα). In specific, non-limiting examples, the 721.221-mIL-21 cell or 721.221-mIL21-B7H6 cell is further transduced or transfected with a heterologous nucleic acid encoding IL-15Rα. In some examples, the nucleic acid encoding IL-15Rα includes or consists of a nucleic acid with at least 90% identity (such as at least 90%, 91%, 92%, 93%, 94%, 95%, 96%, 97%, 98%, 99%, or 100% identity) to SEQ ID NO: 4 and/or encodes a protein including or consisting of an amino acid sequence with at least 95% identity (such as at least 95%, 96%, 97%, 98%, 99%, or 100% identity) to SEQ ID NO: 5. In further, non-limiting examples, the 721.221-mIL-21 cell or 721.221-mIL21-B7H6 cell is further transduced or transfected with heterologous nucleic acids encoding membrane-bound ICAM-1, Fc receptor, CD8U, ULBP-1, ULPB-2, or MIC-A.

The 721.221-mIL-21 cells disclosed herein can be produced, for example, by transfecting 721.221 cells or by transducing 721.221 cells with one or more vectors (such as a lentivirus or retrovirus vector) that includes the heterologous nucleic acid encoding mIL-21. The 721.221-mIL21-B7H6 cells disclosed herein can be produced, for example, by transfecting 721.221 cells or by transducing 721.221 cells with a vector (such as a lentivirus or retrovirus vector) that includes a heterologous nucleic acid encoding mIL-21 and a heterologous nucleic acid encoding B7H6 (which may be included in the same vector or a different vector than that including the nucleic acid encoding mIL-21). In some examples, the 721.221-mIL-21 cells or 721.221-mIL21-B7H6 cells are transformed or are transduced with a vector (such as a lentivirus or retrovirus vector) that includes at least one additional heterologous nucleic acid. For example, the 721.221-mIL-21 cells can be transduced or transfected with at least 1, at least 2, at least 3, at least 4, at least 5, at least 6, at least 7, at least 8, at least 9, at least 10, or more additional heterologous nucleic acids, or about 1-2, 1-3, 1-5, 1-7, or 1-10 additional heterologous nucleic acids, or about 1, 2, or 3 additional heterologous nucleic acids.

Any method of transduction or transfection can be used, such as viral transduction (e.g., using a retrovirus, such as MoMLV or lentivirus) or non-viral transformation, mRNA transfection, or nanoscale nucleic acid delivery (e.g., chemical dendrimers, DNA dendrimers, nanospheres, nanolayers, nanorods, and nanotubes).

In some embodiments, the disclosed methods utilize one or more virus vectors for delivery of the heterologous nucleic acid encoding mIL-21, the heterologous nucleic acid encoding B7-H6 (if present), and optionally at least one additional heterologous nucleic acid, to 721.221 cells. Examples of suitable virus vectors include retrovirus (e.g., MoMLV or lentivirus), adenovirus, adeno-associated virus, vaccinia virus, and fowlpox vectors. In specific examples, a retroviral system is used to introduce the heterologous nucleic acid encoding mIL-21, the heterologous nucleic acid encoding B7-H6 (if present) and optionally at least one additional heterologous nucleic acid, to 721.221 cells. In some examples, a MoMLV vector can be used, such as an SFG retroviral vector. The SFG vector is derived from a murine leukemia virus (MLV) backbone. This type of Murine leukemia virus (MLV)-based retroviral vector is frequently used gene delivery vehicles and has been widely used in clinical trials. Current SFG vectors are fully optimized for gene expression for lymphocyte genetical modification, protein expression, and viral titer.

In some examples, the SFG vector is a gamma retroviral vector that is pseudotyped with the RD114 envelope. RD114 pseudotyped transient retroviral supes can be generated by triple transfection of Peq-Pam plasmid (Moloney GagPol; e.g., at about 4.69 gg), RDF plasmid (RD114 envelope; e.g., at about 3.125 gg), and SFG-VRC01 plasmid (e.g., at about 4.69 gg) into cells (e.g., 293T cells, for example, using GeneJuice (Novagen). Supernatant can be harvested (e.g., after about 48 and 72 hours). High-titer producer lines were generated by multiple transduction of Monkey and Human lymphocytes.

The optional additional one or more heterologous nucleic acid transduced into the 721.221-mIL-21 cells or 721.221-mIL21-B7H6 cells can be a nucleic acid encoding any cytokine, activating receptor ligand, or receptor or fragment thereof, such as IL-15Rα, IL-2, IL-12, IL-33, IL-27, IL-18, IL-7, TLR ligands, ULBP-1, ULBP-2, MIC-A, IL-1 family molecules, Fc receptors, 2B4, ICAM-1, CD8α, CD40, CD28, 4-1BB, 4-1BBL, OX40L, TRX518, CD3 antibody, and/or CD28 antibody. In specific examples, the nucleic acid encodes IL-15Rα, or a combination thereof. In other non-limiting examples, the nucleic acid encodes membrane-bound ICAM-1, Fc receptor, CD8α, ULBP-1, ULPB-2, or MIC-A.

In embodiments where the optional additional at least one heterologous nucleic acid comprises a nucleic acid that encodes a membrane-bound cytokine, the at least one heterologous nucleic acid can comprise a cytokine of interest and additional heterologous nucleic acid sequences (e.g., in the same or separate vector), for example, to form a membrane-bound cytokine. For example, the at least one heterologous nucleic acid can comprise at least one extracellular sequence, at least one transmembrane sequence, and/or at least one intracellular sequence can be used (e.g., in the same vector).

In some examples wherein the 721.221-mIL-21 cells or 721.221-mIL21-B7H6 cells are transduced or transfected with at least one additional heterologous nucleic acid, the at least one additional heterologous nucleic acid comprises at least two extracellular sequences, at least three extracellular sequences, at least four extracellular sequences, or at least five extracellular sequences or about 1-2, 1-3, or 1-5 extracellular sequences. The at least one extracellular sequence can include the cytokine of interest for membrane, such as an additional interleukin. In some examples, the at least one extracellular sequence can include an extracellular fragment from an IgG sequence. In some examples, the at least one extracellular sequence can include an extracellular fragment from a CD8a sequence. In some examples, the at least one heterologous nucleic acid comprises at least two extracellular sequences. In specific examples, the at least two extracellular sequences include an additional cytokine of interest and an extracellular fragment from an IgG sequence.

In some examples, optional additional at least one heterologous nucleic acid comprises at least two transmembrane sequences, or at least three transmembrane sequences or about 1-2 or 1-transmembrane sequences. In some examples, at least one transmembrane sequence can include a transmembrane fragment from a CD28 sequence. Other transmembrane sequences can also be used, such as a transmembrane sequence from CD40L or 2B4. In some examples, at least one heterologous nucleic acid comprises at least two intracellular sequences, at least three intracellular sequences, at least four intracellular sequences, at least five intracellular sequences, or at least six intracellular sequences, or about 1-2, 1-3, or 1-6 intracellular sequences. In some examples, at least one intracellular sequence can include an intracellular fragment from a CD28 sequence, an intracellular fragment from a 4-1BB sequence, and/or an intracellular fragment from a CD3ζ sequence. In some examples, the mIL-21 nucleic acid construct includes or consists of a nucleic acid with at least 90% identity (such as at least 90%, 91%, 92%, 93%, 94%, 95%, 96%, 97%, 98%, 99%, or 100% identity) to SEQ ID NO: 3. In some examples, the mIL-21 nucleic acid construct includes or consists of a nucleic acid encoding an amino acid with at least 90% identity (such as at least 90%, 91%, 92%, 93%, 94%, 95%, 96%, 97%, 98%, 99%, or 100% identity) to SEQ ID NO: 10.

Techniques for the in vitro identification and/or enrichment (e.g., isolation) of 721.221-mIL-21 cells or 721.221-mIL21-B7H6 cells from a broader population (such as a population including 721.221, cells, 721.221-mIL cells, and/or 721.221-mIL21-B7H6 cells) are described herein. In some examples, 721.221-mIL-21 cells, 721.221-mIL21-B7H6 cells, or subsets thereof can be isolated using enriching procedures, such as through the use of immuno-magnetic beads or flow cytometry, such as through fluorescence-activated cell sorting (FACS). For example, detectable antibodies (e.g., by fluorescent or metal labeling) can be used to bind 721.221-mIL-21 cells or 721.221-mIL21-B7H6 cells that express, for example, a surface-expressed cytokine (such as mIL-21 and/or IL-15Rα), TLR ligand, or activating receptor ligand of interest. In FACS analysis, cells are generally marked with fluorescent antibodies, funneled one by one through a flow cytometer, and are sorted. During sorting, cells can be separated into unique populations and each population can be collected into a separate container. In some examples, cells (e.g., a cell population of particular interest, such as a population or subpopulation of 721.221-mIL-21 cells or 721.221-mIL21-B7H6 cells) are isolated using FACS and are then used for further research. For example, isolated 721.221-mIL-21 cells, isolated 721.221-mIL21-B7H6 cells, or isolated subsets thereof can be used in the methods of producing the isolated exosomes disclosed herein.

The 721.221-mIL-21 cells or 721.221-mIL21-B7H6 cells may be grown in a cell culture medium. In one example, the medium is RPMI-1640 (CORNING®). The culture medium can be supplemented with a variety of components useful for optimizing exosome production by and release from the cells. In some examples, the culture medium contains 10% (v/v) fetal bovine serum (FBS) and/or 100 U/mL penicillin-streptomycin (Corning). In other examples, the culture medium is supplemented with 1 ug/mL PMA, 1 ug/mL ionomycin, and/or 1 ug/mL R848, such as for 48-72 hrs, prior to exosome isolation. In some embodiments, 721.221-mIL-21 cells or 721.221-mIL21-B7H6 cells are treated with one or more TLR ligands (such as one or more TLR agonists), prior to exosome isolation. In a specific, non-limiting example, the one or more TLR ligands are LPS, CpG-oligodeoxynucleotides, R848, PHA, PMA, ionomycin, IL-2, and poly(I:C), or a combination or two or more thereof. Exemplary working concentrations of TLR ligands are shown in Table 1.

In some examples, the 721.221-mIL-21 cells or 721.221-mIL21-B7H6 cells are treated with about 0.3 pg/mL to about 7 μg/mL LPS (for example, about 0.5 to about 5 μg/mL LPS), such as about 0.3 μg/mL, 0.4 μg/mL, 0.5 μg/mL, 0.6 μg/mL, 0.7 μg/mL, 0.8 μg/mL, 0.9 μg/mL, 1.0 μg/mL, 1.25 μg/mL, 1.5 μg/mL, 1.75 μg/mL, 2 μg/mL, 2.25 μg/mL, 2.5 μg/mL, 2.75 μg/mL, 3 μg/mL, 3.25 μg/mL, 3.5 μg/mL, 3.75 μg/mL, 4 μg/mL, 4.25 μg/mL, 4.5 μg/mL, 4.75 μg/mL, 5 μg/mL, 5.25 μg/mL, 5.5 μg/mL, 5.75 μg/mL, 6 μg/mL, 6.25 μg/mL, 6.5 μg/mL, 6.75 μg/mL, or about 7 μg/mL LPS.

In some examples, the 721.221-mIL-21 or 721.221-mIL21-B7H6 cells are treated with about 0.1 μg/mL to about 3.5 μg/mL CpG-oligodeoxynucleotides (for example, about 0.24 μg/mL to about 2.4 μg/mL (such as about 0.035 μM to about 0.35 μM) CpG oligodeoxynucleotides), such as about 0.1 μg/mL, 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1.0 μg/mL, 1.1 μg/mL, 1.2 μg/mL, 1.3 μg/mL, 1.4 μg/mL, 1.5 μg/mL, 1.6 μg/mL, 1.7 μg/mL, 1.8 μg/mL, 1.9 μg/mL, 2.0 μg/mL, 2.1 μg/mL, 2.2 μg/mL, 2.3 μg/mL, 2.4 μg/mL, 2.5 μg/mL, 2.6 μg/mL, 2.7 μg/mL, 2.8 μg/mL, 2.9 μg/mL, 3.0 μg/mL, 3.1 μg/mL, 3.2 μg/mL, 3.3 μg/mL, 3.4 μg/mL, or about 3.5 μg/mL CpG-oligodeoxynucleotides.

In some examples, the 721.221-mIL-21 cells or 721.221-mIL21-B7H6 cells are treated with about 0.05 μg/mL to about 1.5 μg/mL R848 (for example, about 0.1 to about 1 μg/mL R848), such as about 0.05 μg/mL, 0.06 μg/mL, 0.07 μg/mL, 0.08 μg/mL, 0.09 μg/mL, 0.1 μg/mL, 0.15 μg/mL, 0.2 μg/mL, 0.25 μg/mL, 0.3 μg/mL, 0.35 μg/mL, 0.4 μg/mL, 0.45 μg/mL, 0.5 μg/mL, 0.55 μg/mL, 0.6 μg/mL, 0.65 μg/mL, 0.7 μg/mL, 0.75 μg/mL, 0.8 μg/mL, 0.85 μg/mL, 0.9 μg/mL, 0.95 μg/mL, 1.0 μg/mL, 1.05 μg/mL, 1.1 μg/mL, 1.15 μg/mL, 1.2 μg/mL, 1.25 μg/mL, 1.3 μg/mL, 1.35 μg/mL, 1.4 μg/mL, 1.45 μg/mL, or about 1.5 μg/mL R848.

In some examples, the 721.221-mIL-21 cells or 721.221-mIL21-B7H6 cells are treated with about 0.5 ng/mL to about 15 ng/mL IL-2 (such as human IL-2, for example, about 1 to about 10 ng/mL human IL-2), such as about 0.5 ng/mL, 0.6 ng/mL, 0.7 ng/mL, 0.8 ng/mL, 0.9 ng/mL, 1.0 ng/mL, 1.5 ng/mL, 2.0 ng/mL, 2.5 ng/mL, 3.0 ng/mL, 3.5 ng/mL, 4 ng/mL, 4.5 ng/mL, 5 ng/mL, 5.5 ng/mL, 6.0 ng/mL, 6.5 ng/mL, 7.0 ng/mL, 7.5 ng/mL, 8.0 ng/mL, 8.5 ng/mL, 9 ng/mL, 9.5 ng/mL, 10 ng/mL, 10.5 ng/mL, 11 ng/mL, 11.5 ng/mL, 12 ng/mL, 12.5 ng/mL, 13 ng/mL, 13.5 ng/mL, 14 ng/mL, 14.5 ng/mL, or about 15 ng/mL IL-2.

In some examples, the 721.221-mIL-21 cells or 721.221-mIL21-B7H6 cells are treated with about 1 μg/mL to about 35 μg/mL PHA (for example, about 2.5 to about 25 μg/mL PHA), such as about 1 μg/mL, 1.5 μg/mL, 2 μg/mL, 2.5 μg/mL, 3 μg/mL, 3.5 μg/mL, 4 μg/mL, 4.5 μg/mL, 5 μg/mL, 6 μg/mL, 7 μg/mL, 8 μg/mL, 9 μg/mL, 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, 20 μg/mL, 21 μg/mL, 22 μg/mL, 23 μg/mL, 24 μg/mL, 25 μg/mL, 26 μg/mL, 27 μg/mL, 28 μg/mL, 29 μg/mL, 30 μg/mL, 31 μg/mL, 32 μg/mL, 33 μg/mL, 34 μg/mL, or about 35 μg/mL PHA.

In some examples, the 721.221-mIL-21 cells or 721.221-mIL21-B7H6 cells are treated with about 0.05 μg/mL to about 1.5 μg/mL PMA (for example, about 0.1 to about 1 μg/mL (such as about 0.162 M to about 1.62 M) PMA), such as about 0.05 μg/mL, 0.06 μg/mL, 0.07 μg/mL, 0.08 μg/mL, 0.09 μg/mL, 0.1 μg/mL, 0.15 μg/mL, 0.2 μg/mL, 0.25 μg/mL, 0.3 μg/mL, 0.35 μg/mL, 0.4 μg/mL, 0.45 μg/mL, 0.5 μg/mL, 0.55 μg/mL, 0.6 μg/mL, 0.65 μg/mL, 0.7 μg/mL, 0.75 μg/mL, 0.8 μg/mL, 0.85 μg/mL, 0.9 μg/mL, 0.95 μg/mL, 1.0 μg/mL, 1.05 μg/mL, 1.1 μg/mL, 1.15 μg/mL, 1.2 μg/mL, 1.25 μg/mL, 1.3 μg/mL, 1.35 μg/mL, 1.4 μg/mL, 1.45 μg/mL, or about 1.5 μg/mL PMA.

In some examples, the 721.221-mIL-21 cells or 721.221-mIL21-B7H6 cells are treated with about 0.05 μg/mL to about 1.5 μg/mL ionomycin (for example, about 0.1 to about 1 μg/mL (such as about 0.134 μM to about 1.34 μM) ionomycin), such as about 0.05 μg/mL, 0.06 μg/mL, 0.07 μg/mL, 0.08 μg/mL, 0.09 μg/mL, 0.1 μg/mL, 0.15 μg/mL, 0.2 p g/mL, 0.25 μg/mL, 0.3 μg/mL, 0.35 μg/mL, 0.4 μg/mL, 0.45 μg/mL, 0.5 p g/mL, 0.55 p g/mL, 0.6 p g/mL, 0.65 p g/mL, 0.7 μg/mL, 0.75 μg/mL, 0.8 μg/mL, 0.85 μg/mL, 0.9 μg/mL, 0.95 μg/mL, 1.0 μg/mL, 1.05 μg/mL, 1.1 μg/mL, 1.15 μg/mL, 1.2 μg/mL, 1.25 μg/mL, 1.3 μg/mL, 1.35 μg/mL, 1.4 μg/mL, 1.45 μg/mL, or about 1.5 μg/mL ionomycin.

In some examples, the 721.221-mIL-21 cells or 721.221-mIL21-B7H6 cells are treated with about 0.05 μg/mL to about 1.5 μg/mL poly(I:C) (for example, about 0.1 to about 1 μg/mL poly(I:C)), such as about 0.05 μg/mL, 0.06 μg/mL, 0.07 μg/mL, 0.08 μg/mL, 0.09 μg/mL, 0.1 μg/mL, 0.15 μg/mL, 0.2 μg/mL, 0.25 μg/mL, 0.3 μg/mL, 0.35 μg/mL, 0.4 μg/mL, 0.45 μg/mL, 0.5 μg/mL, 0.55 μg/mL, 0.6 μg/mL, 0.65 μg/mL, 0.7 μg/mL, 0.75 μg/mL, 0.8 μg/mL, 0.85 μg/mL, 0.9 μg/mL, 0.95 μg/mL, 1.0 μg/mL, 1.05 μg/mL, 1.1 μg/mL, 1.15 μg/mL, 1.2 μg/mL, 1.25 μg/mL, 1.3 μg/mL, 1.35 μg/mL, 1.4 μg/mL, 1.45 μg/mL, or about 1.5 μg/mL poly(I:C).

TLR ligands may be delivered to 721.221-mIL21 cells or 721.221-mIL21-B7H6 cells using a nanoparticle/exosome delivery system. The nanoparticles/exosomes can contain endogenously expressed TLRs or exogenously encapsuled TLRs, or both.

Exosomes may be isolated from a supernatant of a culture of a population of 721.221-mIL-21 cells or 721.221-mIL21-B7H6 cells using various methods known in the art (See, e.g., Kurian et al., Molecular Biotechnology. 63:249-266, 2021, which is incorporated by reference herein in its entirety). Such methods include, but are not limited to, centrifugation (such as ultracentrifugation, such as serial ultracentrifugation), charge neutralization-based precipitation, gel-filtration/size-exclusion chromatography (GF/SEC), immunoaffinity techniques (such as affinity purification using immunogenic beads), ultrafiltration (such as stirred ultrafiltration), double filtration using microfluidic devices, nanoplasmon-enhanced scattering, and lab-on-a-chip devices (such as acoustic nanofiltration, immunoaffinity, filtration, trapping on nanowires, viscoelastic flow sorting, and/or lateral displacement).

In some embodiments, exosomes are isolated from the supernatant of a culture of a population of 721.221-mIL-21 cells or 721.221-mIL21-B7H6 cells using centrifugation, such as serial ultracentrifugation. In some examples, the supernatant of a culture of a population of 721.221-mIL-21 cells or 721.221-mIL21-B7H6 cells is centrifuged in successive rounds with increasing centrifugation forces and durations to remove cells, cellular debris, and/or macromolecular proteins, followed by ultracentrifugation (e.g., at 160,000×g or more for about 50-80 minutes, such as about 50, about 55, about 60, about 65, about 70, about 75, or about 80 min) to obtain isolated exosomes. In some examples, serial ultracentrifugation is used to isolate exosomes from a portion of, substantially all, or all other components of a cell culture supernatant, such as a supernatant from a culture of 721.221-mIL-21 cells or 721.221-mIL21-B7H6 cells.

An exemplary protocol is illustrated in FIG. 2. In a specific, non-limiting example, a supernatant containing the disclosed exosomes is centrifuged at about 250-350×g (such as about 250×g, about 275×g, about 300×g, about 325×g, or about 350×g) for about 3-8 minutes (such as about 3 min, about 4 min, about 5 min, about 6 min, about 7 min, or about 8 min) to remove live cells, then at about 1000-1400×g (such as about 1000×g, about 1050×g, about 1100×g, about 1150×g, about 1200×g, about 1250×g, about 1300×g, about 1350×g, or about 1400×g) for about 15-25 min (such as about 15 min, about 20 min, or about 25 min) to remove dead cells, and then at about 8,000-12,000×g (such as about 8,000×g, about 8,500×g, about 9,000×g, about 9,500×g, about 10,000×g, about 10,500×g, about 11,000×g, about 11,500×g, or about 12,000×g) for about 15-25 min (such as about 15 min, about 20 min, or about 25 min) to remove debris and apoptotic bodies. In such examples, to obtain highly pure concentrated exosomes, the supernatant is further ultracentrifuged at about 100,000-180,000×g (such as about 100,000×g, about 100,500×g, about 110,000×g, about 110,500×g, about 115,000×g, about 120,000×g, about 125,000×g, about 130,000×g, about 135,000×g, about 140,000×g, about 145,000×g, about 150,000×g, about 155,000×g, about 160,000×g, about 165,000×g, about 170,000×g, about 175,000×g, or about 180,000×g) for about 50-70 min (such as about 50 min, about 55 min, about 60 min, about 65 min, or about 70 min) and then washed, such as with PBS, for example twice. A final exosome pellet may be resuspended, such as in PBS, for example in 1 mL of 1×PBS. In some embodiments, the isolated exosomes (such as the exosome pellet, such as the exosome pellet resuspended in PBS) is further filtered prior to use in the disclosed methods (such as in a method of expanding a population of NK cells or T cells using the isolated exosomes). In a specific, non-limiting example, the isolated exosomes are passed through a membrane, such as a 0.22-μm filtration membrane, prior to use in the disclosed methods.

Isolated exosomes can be quantified using a variety of methods known in the art (See, e.g., Kurian et al., Molecular Biotechnology. 63:249-266, 2021). Such methods include, but are not limited to nanoparticle tracking analysis, flow cytometry, tunable resistive pulse sensing, electron microscopy, mass spectrometry (for example, to quantify exosomes based on the level of one or more proteins known to be present in the exosomes), dynamic light scattering, and microfluidic devices. For example, exosomes can be quantified using commercially available kits, such as the NanoSight NS300 Exosome Quantitation Kit (System Biosciences, Palo Alto, CA, USA).

Exosomes can be identified and characterized using various assays known in the art, including confocal microscopy (including STED super-resolution imaging system) and/or electron microscopy (EM, which allows for the determination of particle sizes and therefore can be used to distinguish between exosomes and other vesicles). The sizes of the isolated exosomes disclosed herein can be measured using, for example, NanoSight assay (optical microscopy techniques adapted to quantify small particles such as exosomes). This assay utilizes Nanoparticle Tracking Analysis (NTA) to characterize nanoparticles from 10-1000 nm in size. Concentrations of proteins in and on the disclosed isolated exosomes can be measured using various assays known in the art, such as, but not limited to, bicinchoninic acid (BCA) assay and/or ExoELISA (System Biosciences; an ELISA kit specific to quantification of exosome particles). Further, proteins in and on the disclosed exosomes can be identified using various methods known in the art, such as mass spectrometry.

The disclosed isolated exosomes may include one or more, such as 1-50 or more (such as 1-10, 2-20, 5-30, 10-40, or 20-50, such as 1, 2, 3, 4, 5, 6, 7, 8, 9, 10, 11, 12, 13, 14, 15 or more) molecules that can stimulate immune cell expansion, such as expansion of NK cells, T cells, macrophages, NKT cells, CAR-NK cells, CAR-T cells, CAR-M cells, or CAR-NKT cells.

In some embodiments, isolated exosomes useful in the disclosed methods can include one or more of mIL-21, 4-1BB, one or more KIR ligands (such as HLA-C2, HLA-C1, HLA-Bw4, HLA-G, HLA-A, and/or HLA-F), one or more MHC class I molecules (such as HLA-A, HLA-B, and/or HLA-C), one or more MHC class II molecules (such as HLA-DP, HLA-DM, HLA-DOA, HLA-DOB, HLA-DQ, and/or HLA-DR), or a combination thereof. In additional embodiments, isolated exosomes useful in the disclosed methods can include one or more (such as 1, 2, 3, 4, 5, or more, for example 1-5, 2-10, 5-15, 15-25, 25-40, 30-50, or more) of the proteins listed in Table 2.

### III. Methods of Expanding Immune Cells

Disclosed herein are methods of expanding a population of immune cells (such as a population of NK cells, T cells, macrophages, or NKT cells). Such methods include contacting the population of immune cells with an isolated exosome disclosed herein (such as contacting the population of immune cells with an effective amount of the disclosed isolated exosomes), under conditions sufficient for cell population expansion. In particular examples, the methods disclosed herein are utilized to expand CAR-modified NK cells, CAR-modified T cells, CAR-modified macrophages, or CAR-modified NKT cells.

Also disclosed herein are methods of expanding a population of immune cells (such as a population of NK cells, T cells, macrophages, or NKT cells) using the modified 721.221-mIL21-B7H6 cells provided herein. In some examples, to enhance expansion, the immune cells are expanded with the 721.221-mIL21-B7H6 cells. The 721.221-mIL21-B7H6 cells disclosed herein may be utilized as feeder cells for the immune cells (such as NK or T cells). Any amount of cells for expansion and feeders cells can be used. In some examples, the amount of cells for expansion (e.g., PMBCs) can include at least about 10¹, at least about 10², at least about 10³, at least about 10⁴, at least about 10⁵, at least about 10⁶, at least about 10⁷, at least about 10⁸, at least about 10⁹, or at least about 10¹⁰, about 10¹-10¹⁰ , 10⁴-10⁸, or about 10⁶, such as 5×10⁶ cells. In some examples, the cells for expansion (e.g., a population of cells comprising NK cells or T cells, such as PMBCs) can be contacted with at least about 10¹, at least about 10², at least about 10³, at least about 10⁴, at least about 10⁵, at least about 10⁶, at least about 10⁷, at least about 10⁸, at least about 10⁹, or at least about 10¹⁰, about 10¹-10¹⁰, 10⁵-10⁹, or about 10⁶, such as 1×10⁷ cells feeder cells (e.g., modified 721.221 cells, for example, 721.221 cells expressing mIL-21 and B7-H6). In some examples, the ratio of cells for expansion (e.g., PMBCs) to the feeder cells can be at least about 1:1 to about 1:50, for example, at least about 1:1, at least about 1:2, at least about 1:5, at least about 1:6, at least about 1:7, at least about 1:8, at least about 1:9, at least about 1:10, at least about 1:15, at least about 1:20, at least about 1:25, at least about 1:30, at least about 1:35, at least about 1:40, at least about 1:45, or at least about 1:50 or about 1:2, about 1:7, about 3:20, or about 1:20. In some examples, further reagents are used to enhance expansion, such as additional cytokines, for example, IL-2, IL-5, IL-7, IL-8, and/or IL-12. The cells for expansion (e.g., a population of cells comprising NK cells or T cells, such as PMBCs) are contacted with 721.221-mIL21-B7H6 cells feeder cells and/or other expansion-enhancing reagents (e.g., IL-2, IL-5, IL-7, IL-8, and/or IL-12) for at least about 1-40 days, such as at least about 1, at least about 3, at least about 5, at least about 7, at least about 10, at least about 14, at least about 21, at least about 28, at least about 35, about 10-30, 10-20, 20-30, or 15-25, or about 14 days (e.g., for T cell expansion) or about 21 days (e.g., for NK cell expansion, such as CAR-NK cells).

Techniques for the in vitro or ex vivo isolation and enrichment of immune cells are described herein. Exemplary procedures are described in US Pat. App. Publ. No. 2014/0086890, WO Pat. Pub. No. 2017/127729, and US Pat. Pub. No. 2013/0315884 incorporated herein by reference in their entireties. One of ordinary skill in the art can identify additional methods for expanding NK or T cells, for example, as described in Childs et al., Hematol. The Education Program 2013:234-246, 2013; U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005 incorporated herein by reference in their entireties.

Mononuclear cells are collected from a subject (such as a healthy subject, a donor subject, or a subject with a cancer, immune disorder, or infectious disease) or from a donor HLA-matched to the subject to be treated. In some examples, the mononuclear cells are autologous to the subject, such as to the subject having a cancer or infectious or immune disease to be treated. In some examples, mononuclear cells are collected by an apheresis procedure. The mononuclear cells are enriched for NK cells, T cells, macrophages, or NKT cells, for example, by negative depletion using an immuno-magnetic bead strategy. In other examples, the mononuclear cells comprise PMBCs, for example, isolated using a polysaccharide technology, such as a Ficoll®-based separation method (GE® Healthcare).

In some examples, NK cells are optionally enriched by depleting the mononuclear cell sample of non-NK cells (such as T cells, B cells, monocytes, dendritic cells, platelets, macrophages, and erythrocytes) utilizing a mixture of biotinylated monoclonal antibodies. In some examples, the non-NK cells in the sample are removed with magnetic beads coupled to streptavidin, resulting in an enriched preparation of NK cells. An exemplary commercially available kit for this method is Dynabeads® Untouched™ Human NK Cells kit (ThermoFisher Scientific, Waltham, MA).

In some examples, T cells are enriched by depleting the mononuclear cell sample of non-T cells (such as NK cells, B cells, monocytes, dendritic cells, platelets, macrophages, and erythrocytes) utilizing a mixture of biotinylated monoclonal antibodies. In some examples, the non-T cells in the sample are removed with magnetic beads coupled to streptavidin, resulting in an enriched preparation of T cells. An exemplary commercially available kit for this method is EASYSEP™ Human T Cell Isolation Kit (STEMCELL™ technologies, Cambridge, MA). In some examples, the non-T cells in the sample are removed with magnetic beads coupled to streptavidin, resulting in an enriched preparation of T cells. T cells enriched in this fashion are also known herein as purified T cells.

In some examples, NKT cells are optionally enriched by depleting the mononuclear cell sample of non-NKT cells (such as NK cells, other T cells, B cells, monocytes, dendritic cells, platelets, macrophages, and erythrocytes) utilizing a mixture of biotinylated monoclonal antibodies. In some examples, the non-NKT cells in the sample are removed with magnetic beads coupled to streptavidin, resulting in an enriched preparation of NKT cells. An exemplary commercially available kit for this method is the human CD3+CD56+ NKT Cell Isolation Kit (Miltenyi Biotec, San Diego, CA).

In some examples, monocytes for producing a population of macrophages are optionally enriched by depleting the mononuclear cell sample of non-monocyte cells (such as NK cells, T cells, B cells, dendritic cells, platelets, macrophages, and erythrocytes) utilizing a mixture of biotinylated monoclonal antibodies. In some examples, the monocytes are isolated from PBMCs using plastic adhesion, negative, or CD14 positive selection. In some examples, the non-monocyte cells in the sample are removed with magnetic beads coupled to streptavidin, resulting in an enriched preparation of monocyte cells. An exemplary commercially available kit for this method is the EASYSEP™ Human Monocyte Enrichment Kit (ThermoFisher Scientific, Waltham, MA). Monocytes isolated from PBMCs can be matured to monocyte-derived macrophages using, for example, M-CSF and/or GM-CSF, and/or other cytokines and chemokines (such as IL-2, IL-13, IL-17, IL-21, IL-1β, IL-6, IFN-γ, and/or TNF-α).

In some examples, NK cells, T cells, macrophages, or NKT cells are enriched by positive selection. In some examples, the methods include enriching for NK cells, such as by positive selection of CD56⁺ NK cells, for example utilizing magnetic beads conjugated to an anti-CD56 antibody (such as CD56 MicroBeads, Miltenyi Biotec, Inc., Auburn, CA). In other examples, a two-step method including negative depletion (such as T cell depletion) followed by positive selection of CD56⁺ NK cells is used for enriching NK cells. In other examples, the methods include enriching for T cells, such as by positive selection of CD4⁺ T cells or CD8⁺ T cells, for example utilizing magnetic beads conjugated to an anti-CD4 or anti-CD8 antibody (such as CD4 or CD8 MicroBeads, Miltenyi Biotec, Inc., Auburn, CA). In other examples, a two-step method including negative depletion (such as NK cell depletion) followed by positive selection of CD4⁺ T cells or CD8⁺ T cells is used for enriching T cells. One of ordinary skill in the art can identify other methods that can be used to prepare an enriched population of NK or T cells. For example, NK cells can be also isolated from various tissues (e.g., cord blood, liver, lung tissues, and similar) using commercially available NK cell isolation kits. Populations of NK cells, T cells, macrophages, or NKT cells enriched as described herein are also known herein as purified NK (pNK) cells, purified T cells, purified macrophages, or purified NKT cells, respectively.

The isolated NK cells, T cells, macrophages, or NKT cells can be analyzed by flow cytometry for the expression of markers. In some examples, the markers can be used to assay for purity of the isolated cells. In some examples, CD56 can be used as a marker, for example, to analyze NK cells. In some examples, CD8 or CD4 can be used as a marker, for example, to analyze T cells. In some examples, CD14, CD16, CD64, CD68, CD71 or CCR5 can be used as a marker, for example, to analyze macrophages. In some examples, CD3 and CD56 co-expression can be used as a marker, for example, to analyze NKT cells.

In some embodiments, NK cells, T cells, macrophages, or NKT cells are expanded in vitro. In some examples, enriched NK cells, T cells, macrophages, or NKT cells can be used for expansion. In other examples, NK cells, T cells, macrophages, or NKT cells are expanded using a heterogeneous pool of cells, such as a population of cells derived from a sample, such as a tissue, fluid, or blood sample. In some examples, the population of cells comprises peripheral blood mononuclear cells (PMBCs). The population of cells (e.g., PMBCs) can be generated from any tissue, fluid, or blood sample can be used, for example, peripheral blood, cord blood, ascites, menstrual blood, or bone marrow. In specific examples, the population of cells comprises PBMCs from healthy donors, cord blood mononuclear cells from healthy donors, or PBMCs from a subject with cancer.

In some embodiments of the disclosed methods, a population of NK cells, T cells, macrophages, or NKT cells are expanded using the isolated exosomes disclosed herein. Any amount (such as an effective amount) of immune cells for expansion and any amount of isolated exosomes can be used in the disclosed methods. In some examples, the amount of cells for expansion (e.g., PMBCs, pNK cells, purified T cells, purified NKT cells, or purified macrophages (such as monocyte-derived macrophages)) can include at least about 10¹, at least about 10², at least about 10³, at least about 10⁴, at least about 10⁵, at least about 10⁶, at least about 10⁷, at least about 10⁸, at least about 10⁹, or at least about 10¹⁰, about 10¹-10¹⁰, 10⁴-10⁸, or about 10⁶, such as 5×10⁶ cells. In some examples, the cells for expansion (e.g., a population of cells comprising NK cells or T cells, such as PMBCs, pNK cells, or purified T cells) can be contacted with at least about 10¹, at least about 10², at least about 10³, at least about 10⁴, at least about 10⁵, at least about 10⁶, at least about 10⁷, at least about 10⁸, at least about 10⁹, or at least about 10¹⁰, about 10¹-10¹⁰, 10⁵-10⁵, or about 10⁶, such as 1×10⁷ isolated exosomes.

In some examples, the ratio of cells for expansion (e.g., PMBCs, pNK cells, purified T cells, purified NKT cells, or purified macrophages (such as monocyte-derived macrophages)) to the isolated exosomes can be at least about 1:1 to about 1:50, for example, at least about 1:1, at least about 1:2, at least about 1:5, at least about 1:6, at least about 1:7, at least about 1:8, at least about 1:9, at least about 1:10, at least about 1:15, at least about 1:20, at least about 1:25, at least about 1:30, at least about 1:35, at least about 1:40, at least about 1:45, or at least about 1:50 or about 1:2, about 1:7, about 3:20, or about 1:20. In some examples, further reagents are used to enhance expansion, such as additional cytokines, for example, IL-2, IL-5, IL-7, IL-8, and/or IL-12.

The cells for expansion (e.g., a population of cells comprising NK cells, T cells, macrophages, or NKT cells, such as PMBCs, pNK cells, purified T cells, purified NKT cells, or purified macrophages (such as monocyte-derived macrophages)) are contacted with isolated exosomes (with or without other expansion-enhancing reagents (e.g., IL-2, IL-5, IL-7, IL-8, and/or IL-12)) for at least about 1-40 days, such as at least about 1, at least about 3, at least about 5, at least about 7, at least about 10, at least about 14, at least about 21, at least about 28, at least about 35, about 10-30, about 10-20, about 20-30, about 15-25, or about 14 days (e.g., for T cell expansion, such as CAR-T cell expansion) or about 21 days (e.g., for NK cell expansion, such as CAR-NK cell expansion). In some embodiments, the exosomes are added to the culture medium of the population of cells to be expanded. In some embodiments, the exosomes are added to the culture medium of the population of cells one time. In other embodiments, the exosomes are added to the culture medium of the population of cells more than once, such as 2-5 times, such as 2, 3, 4, or 5 times over the course of the culture.

The expanded NK cells, T cells, macrophages, or NKT cells (e.g., enriched or in a heterogeneous population of cells, such as PMBCS) produced using the techniques disclosed herein (e.g., by contacting the NK cells, T cells, macrophages, or NKT cells with the isolated exosomes can be superior to control expansion techniques, where the isolated exosomes are not used (for example, as compared to methods where the cells are expanded in the presence of feeder cells). In some examples, expansion using the techniques disclosed herein can enhance expansion by about 25-fold to at least about 2,000-fold, such as at least about 25-fold, at least about 30-fold, at least about 40-fold, at least about 50-fold, at least about 60-fold, at least about 70-fold, at least about 80-fold, at least about 90-fold, at least about 10⁰-fold, at least about 150-fold, at least about 200-fold, at least about 250-fold, at least about 300-fold, at least about 350-fold, at least about 400-fold, at least about 450-fold, at least about 500-fold, at least about 550-fold, at least about 600-fold, at least about 650-fold, at least about 700-fold, at least about 750-fold, at least about 800-fold, at least about 850-fold, at least about 900-fold, at least about 950-fold, at least about 1,000-fold, at least about 1,100-fold, at least about 1,150-fold, at least about 1,200-fold, at least about 1,250-fold, at least about 1,300-fold, at least about 1,350-fold, at least about 1,400-fold, at least about 1,450-fold, at least about 1,500-fold, at least about 1,550-fold, at least about 1,600-fold, at least about 1,650-fold, at least about 1,700-fold, at least about 1,750-fold, at least about 1,800-fold, at least about 1,850-fold, at least about 1,900-fold, at least about 1,950-fold, or at least about 2,000-fold (for example, as compared to methods where the cells are expanded in the presence of feeder cells). In other examples, expansion using the techniques disclosed herein can enhance expansion by about 2,000-fold to at least about 10^{0,000}-fold, such as at least about 2,000-fold, at least about 5,000-fold, at least about 10^{,000}-fold, at least about 15,000-fold, at least about 20,000-fold, at least about 25,000-fold, at least about 30,000-fold, at least about 35,000-fold, at least about 40,000-fold, at least about 45,000-fold, at least about 50,000-fold, at least about 55,000-fold, at least about 60,000-fold, at least about 65,000-fold, at least about 70,000-fold, at least about 75,000-fold, at least about 80,000-fold, at least about 85,000-fold, at least about 90,000-fold, at least about 95,000-fold, or at least about 100,000-fold.

In some examples, cytotoxicity of the expanded NK cells, T cells, macrophages, or NKT cells can be evaluated. Cytotoxicity can be evaluated at any time, such as after the NK cells, T cells, macrophages, or NKT cells are expanded or, optionally, the expanded NK cells, T cells, macrophages, or NKT cells can be transduced (for example, to express one or more chimeric antigen receptors (CARs)). In some examples, to evaluate cytotoxicity against tumor cells, animal models can be used, such as animal models expressing a detectable tumor marker (e.g., a bioluminescent tumor marker, such as luciferase, for example, FFluc-Daudi tumor cells). In other examples, cytotoxicity is evaluated using cell culture methods, such as against tumor cells in culture, such as against K562 cells. In specific examples, the NK cells, T cells, macrophages, or NKT cells exhibit improved cytotoxicity, for example, against tumor cells, compared with control NK cells, T cells, macrophages, or NKT cells produced without the methods disclosed herein. In some examples, the NK cells, T cells, macrophages, or NKT cells expanded using the disclosed exosomes (the NFC expansion system) exhibit at least similar cytotoxicity as compared to NK cells, T cells, macrophages, or NKT cells expanded using a FC system. In some examples, the NK cells, T cells, macrophages, or NKT cells produced using the disclosed methods can exhibit greater cytotoxicity, for example, against tumor cells, by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, or at least about 10-fold, about 0.5-10-fold, 1-5-fold, or 5-10-fold, or about 3-fold greater toxicity, for example, compared to cells expanded using cytokines only. In other examples, the NK cells, T cells, macrophages, or NKT cells produced using the disclosed methods can exhibit greater cytotoxicity (such as against tumor cells) by at least about 11-fold, at least about 12-fold, at least about 15-fold, at least about 20-fold, at least about 25-fold, at least about 30-fold, at least about 35-fold, at least about 40-fold, at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 95-fold, or at least about 100-fold greater toxicity (such as compared to cells expanded using cytokines only). In some examples, chromium release assays can be used to assess NK cell cytotoxicity against cell targets. One of ordinary skill in the art can identify other methods to assess the isolated NK cell population (for example, NK cell purity, viability, and/or activity).

In some embodiments, the NK cells, T cells, macrophages, or NKT cells can be further transfected or transduced to express a protein of interest. In specific examples, the NK cells, T cells, macrophages, or NKT cells can be transfected or transduced to express at least one CAR. The modified NK cells, T cells, macrophages, or NKT cells (such as the CAR-NK, CAR-T, CAR-M, or CAR-NKT cells) can then be expanded using the exosomes and methods disclosed herein. The NK cells, T cells, macrophages, or NKT cells can be transfected or transduced at any time throughout the methods described herein, such as before expansion or during expansion. In specific examples, the NK cells, T cells, macrophages, or NKT cells can be transfected or transduced with at least one CAR during expansion, for example, after at least about ¼, ⅓, ½, or ¾ of the duration of the expansion process. In other examples, NK cells, T cells, macrophages, or NKT cells expanded using the exosomes disclosed herein are subsequently modified to express at least one CAR. In particular examples, the CAR is CD19, such as CD19 CAR-NK cells. In other examples, the CAR is CD147, such as CD147 CAR-NK cells.

In specific examples, the NK cells, T cells, macrophages, or NKT cells can be transduced with viral vectors comprising at least one CAR of interest for delivery therein, such as a heterologous nucleic acid comprising at least one CAR (and optionally other components). Examples of suitable virus vectors include retrovirus (e.g., MoMLV or lentivirus), adenovirus, adeno-associated virus, vaccinia virus, and fowlpox vectors. In specific examples, a retroviral system is used to introduce the at least one CAR into NK cells, T cells, macrophages, or NKT cells. In some examples, a MoMLV vector can be used, such as an SFG retroviral vector. In some examples, the at least one CAR can comprise a protein or fragments thereof from at least one transmembrane sequence and/or at least one intracellular sequence (e.g., in the same or different vectors).

In some examples, the at least one heterologous nucleic acid comprises at least two extracellular sequences, at least three extracellular sequences, at least four extracellular sequences, or at least five extracellular sequences or about 1-2, 1-3, or 1-5 extracellular sequences. The at least one extracellular sequence can include any CAR(s) of interest, such as a CD19 or kappa light chain sequence. In some examples, at least one extracellular sequence can include an extracellular fragment from an IgG sequence. Other extracellular sequences can be used, including extracellular sequences from CD8a or CD28. In some examples, the at least one heterologous nucleic acid comprises at least two extracellular sequences. In specific examples, the at least two extracellular sequences include a CAR of interest, such as CD19 or kappa, and an extracellular fragment from an IgG sequence.

In some examples, the at least one heterologous nucleic acid comprises at least two transmembrane sequences, or at least three transmembrane sequences or about 1-2 or 1-transmembrane sequences. In some examples, the at least one transmembrane sequence can include a transmembrane fragment from a CD28 sequence. Other transmembrane sequences can be used, such as a 4-1BB sequence. In some examples, the at least one heterologous nucleic acid comprises at least two intracellular sequences, at least three intracellular sequences, at least four intracellular sequences, at least five intracellular sequences, or at least six intracellular sequences, or about 1-2, 1-3, or 1-6 intracellular sequences. In some examples, the at least one intracellular sequence can include an intracellular fragment from a CD28 sequence, an intracellular fragment from a 4-1BB sequence, and/or an intracellular fragment from a CD3ζ sequence.

Additional CARs can be used, for example, LL1 (anti-CD74), GD2 antigen, CD5 antigen, CD57 antigen, LL2 or RFB4 (anti-CD22), veltuzumab (hA20, anti-CD20), rituxumab (anti-CD20), obinutuzumab (GA101, anti-CD20), lambrolizumab (anti-PD1), nivolumab (anti-PD1), MK-3475 (anti-PD1), AMP-224 (anti-PD1), pidilizumab (anti-PD1), MDX-1105 (anti-PD-LI), MEDI4736 (anti-PD-Ll), MPDL3280A (anti-PD-LI), BMS-936559 (anti-PD-LI), ipilimumab (anti-CTLA4), trevilizumab (anti-CTL4A), RS7 (anti-epithelial glycoprotein-1 (EGP-1, also known as TROP-2)), PAM4 or KC4 (both anti-mucin), MN-14 (anti-carcinoembryonic antigen (CEA, also known as CD66e or CEACAM-5), MN-15 or MN-3 (anti-CEACAM-6), Mu-9 (anti-colon-specific antigen-p), Immu 31 (an anti-alpha-fetoprotein), RI (anti-IGF-lR), A19 (anti-CD19), TAG-72 (e.g., CC49), Tn, 1591 or HuJ591 (anti-PSMA (prostate-specific membrane antigen)), AB-PGl-XGl-026 (anti-PSMA dimer), D2/B (anti-PSMA), G250 (an anti-carbonic anhydrase IX MAb), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin), BWA-3 (anti-histone H2A/H4), LG2-1 (anti-histone H3), MRA12 (anti-histone H1), PRI-1 (anti-histone H2B), LGll-2 (anti-histone H2B), LG2-2 (anti-histone H2B), and trastuzumab (anti-ErbB2), carbonic anhydrase IX, B7, CCL19, CCL21, CSAp, HER-2/neu, BrE3, CDI, CDIa, CD2, CD3, CD4, CDS, CDS, CDIIA, CD14, CD15, CD16, CD18, CD19, CD20 (e.g., C2B8, hA20, 1F5 MAbs), CD21, CD22, CD23, CD25, CD29, CD30, CD32b, CD33, CD37, CD38, CD40, CD40L, CD44, CD45, CD46, CD47, CD52, CD54, CD55, CD59, CD64, CD67, CD70, CD72, GPC-3, CD74, CD79a, CDSO, CD83, CD95, CD126, CD133, CD137, D138, CD147, CD154, CD127 (also known as B7-H3), CEACAM-5, CEACAM-6, CTLA4, alpha-fetoprotein (AFP), VEGF (e.g., AVASTIN®, fibronectin splice variant), ED-B fibronectin (e.g., L19), EGP-1 (TROP-2), EGP-2 (e.g., 17-IA), EGF receptor (ErbBI) (e.g., ERBITUX®), ErbB2, ErbB3, Factor H, FHL-1, Flt-3, folate receptor, Ga 733, GRO family proteins, HMGB-1, hypoxia inducible factor (HIF), HMl 0.24, HER-2/neu, insulin-like growth factor (ILGF), IFN family proteins, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-lR, Ia, HM1.24, ganglio-sides, Fas-L, HCG, the HLA-DR antigen to which L243 binds, CD66 antigens (i.e., CD66a-d or a combination thereof), MAGE, mCRP, MCP-1, MIP-1A, MIP-18, macrophage migration-inhibitory factor (MIF), MUC1, MUC2, MUC3, MUC4, MUC5ac, placental growth factor (PlGF), PSA (prostate-specific antigen), PSMA, PAM4 antigen, PD1 receptor, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), TROP-2, VEGFR, RANTES, and TI0l. Other CARs are possible, such as multispecific CARs (e.g., bispecific or trispecific CARs, such as including one or more CAR disclosed herein).

### IV. Methods of Treating a Disease or Disorder Utilizing the Expanded Immune Cells

Disclosed herein are methods of treating a subject with a disease or disorder by administering NK cells, T cells, macrophages, or NKT cells (e.g., CAR-modified NK cells, T cells, macrophages, or NKT cells) expanded using the methods described herein (such as an effective amount of the immune cells expanded using the methods described herein) to the subject. In specific, non-limiting examples, NK cells are administered. The non-modified NK cells, T cells, macrophages, or NKT cells or modified (e.g., CAR-modified) NK cells, T cells, macrophages, or NKT cells described herein can be administered either to animals or to human subjects. In particular examples, the NK cells, T cells, macrophages, or NKT cells (or CAR-NK, CAR-T, CAR-M, or CAR-NKT cells) are from a non-HLA matched donor, including an unrelated individual, or an HLA-matched donor. In other examples, the NK cells, T cells, macrophages, or NKT cells (or CAR-NK, CAR-T, CAR-M, or CAR-NKT cells) are from the subject being treated (e.g., are autologous).

In some embodiments, the disease or disorder is a cancer (e.g., solid cancer (such as sarcomas (e.g., rhabdomyosarcoma, osteogenic sarcoma, Ewing's sarcoma, chondrosarcoma, and alveolar soft part sarcoma); carcinomas (e.g., colorectal carcinoma); and lymphomas, such as Hodgkin's or non-Hodgkin's lymphoma, for example, diffuse large B-cell, follicular, chronic lymphocytic, small lymphocytic, mantle cell, Burkitt's, cutaneous T-cell, AIDS-related, or central nervous system lymphoma); neuroblastoma; gynecological cancer (such as uterine or ovarian cancer); breast cancer; liver cancer; lung cancer; prostate cancer; skin cancer; bone cancer; pancreatic cancer; brain cancer (neuroblastoma or glioma); head or neck cancer; kidney cancer (such as Wilms' tumor); retinoblastoma; adrenocortical tumor; desmoid tumors; desmoplastic small round cell tumor; endocrine tumors; and/or blood cancer (such as myeloma, such as multiple myeloma; lymphoma, such as Hodgkin's or non-Hodgkin's lymphoma, for example, diffuse large B-cell, follicular, chronic lymphocytic, small lymphocytic, mantle cell, Burkitt's, cutaneous T-cell, AIDS-related, or central nervous system lymphoma; or leukemia, such as acute lymphocytic leukemia (ALL) or acute myeloid leukemia (AML))), an immune disorder (e.g., an autoimmune disorder or transplant rejection), or an infectious disease (for example, cytomegalovirus, adenovirus, respiratory syncytial virus, Epstein-Barr virus, HIV infection, or hepatitis virus infection (such as hepatitis C virus or hepatitis B virus)).

The expanded NK cells, T cells, macrophages, or NKT cells produced as described herein can be incorporated into pharmaceutical compositions. Such compositions typically include a population of NK cells, T cells, macrophages, or NKT cells (such as modified NK cells, T cells, macrophages, or NKT cells, such as CAR-NK, CAR-T, CAR-M, or CAR-NKT cells) and a pharmaceutically acceptable carrier. A “pharmaceutically acceptable carrier” includes any and all solvents, diluents, dispersion media, coatings, antibacterial and antifungal agents, isotonic and absorption delaying agents, and the like, compatible with pharmaceutical administration (see, e.g., Remington: The Science and Practice of Pharmacy, The University of the Sciences in Philadelphia, Editor, Lippincott, Williams, & Wilkins, Philadelphia, PA, 21^{st} Edition, 2005). Examples of such carriers include, but are not limited to, water, saline, Ringer's solutions, dextrose solution, balanced salt solutions, and 5% human serum albumin. Liposomes and non-aqueous vehicles such as fixed oils may also be used. Supplementary active compounds can also be incorporated into the compositions. Actual methods for preparing administrable compositions are known or apparent to those skilled in the art and are described in more detail in such publications as Remington: The Science and Practice of Pharmacy, The University of the Sciences in Philadelphia, Editor, Lippincott, Williams, & Wilkins, Philadelphia, PA, 21^{st} Edition (2005). In one non-limiting example, the expanded cells are suspended in PLASMA-LYTE™ multiple electrolyte solution.

In some examples, an effective amount of immune cells (such as NK cells, CAR-NK cells, T cells, CAR-T cells, macrophages, CAR-M cells, NKT cells, or CAR-NKT cells) expanded as disclosed herein can be administered to a subject. In some examples, about 10⁴ to 10¹² of the NK cells, CAR-NK cells, T cells, CAR-T cells, macrophages, CAR-M cells, NKT cells, or CAR-NKT cells (for example, about 10⁴-10⁸ cells, about 10⁶-10⁸ cells, or about 10⁶-10¹² cells) are administered to the subject. For example, about 10⁴ to 10¹⁰ NK cells, CAR-NK cells, T cells, CAR-T cells, macrophages, CAR-M cells, NKT cells, or CAR-NKT cells per kg bodyweight (such as about 10⁴, 10⁵, 10⁶, 10⁷, or 10⁸ cells/kg) are administered to a subject. In specific examples, at least 10⁴, 10⁵, 10⁶, or 10⁷ NK cells, CAR-NK cells, T cells, CAR-T cells, macrophages, CAR-M cells, NKT cells, or CAR-NKT cells are administered to the subject. The population of NK cells, CAR-NK cells, T cells, CAR-T cells, macrophages, CAR-M cells, NKT cells, or CAR-NKT cells is typically administered parenterally, for example intravenously; however, injection or infusion to a cancer (e.g., a tumor) or close to a cancer (local administration) or administration to the peritoneal cavity can also be used. One of skill in the art can determine appropriate routes of administration.

Multiple doses of the population of NK cells, T cells, macrophages, or NKT cells (such as the CAR-modified cells) can be administered to a subject. For example, the population of NK cells, T cells, macrophages, or NKT cells (such as the CAR-NK, CAR-T, CAR-M, or CAR-NKT cells) can be administered daily, every other day, twice per week, weekly, every other week, every three weeks, monthly, or less frequently. A skilled clinician can select an administration schedule based on the subject, the condition being treated, the previous treatment history, and other factors.

In additional examples, the subject is also administered at least one, at least one, at least two, at least three, or at least four cytokine(s) (such as IL-2, IL-15, IL-21, and/or IL-12) to support survival and/or growth of the NK cells, T cells, macrophages, or NKT cells (such as the CAR-NK, CAR-T, CAR-M, or CAR-NKT cells). In specific, non-limiting examples, at least one cytokine includes IL-2 and IL-15 (e.g., to support survival and/or growth of NK cells). The cytokine(s) are administered before, after, or substantially simultaneously with the NK cells, T cells, macrophages, or NKT cells. In specific examples, at least one (e.g., IL-2 and/or IL-2) is administered simultaneously, for example, with NK cells (such as CAR-NK cells, such as CD19 CAR-NK cells or CD147 CAR-NK cells).

In some examples, the methods include treating or inhibiting cancer, such as a hematological malignancy or a solid tumor. Examples of hematological malignancies include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), T-cell large granular lymphocyte leukemia, polycythemia vera, lymphoma, Hodgkin's lymphoma, non-Hodgkin's lymphoma (indolent and high grade forms; includes diffuse large B-cell, follicular, chronic lymphocytic, small lymphocytic, mantle cell, Burkitt's, cutaneous T-cell, AIDS-related, or central nervous system lymphoma), multiple myeloma, Waldenstrom's macroglobulinemia, heavy chain disease, myelodysplastic syndrome, hairy cell leukemia, and myelodysplasia. Unmodified or modified (e.g., CAR-modified) NK or T cells can be administered. In specific examples, unmodified NK or T cells expanded using the methods herein can be administered to treat or inhibit lymphoma, such as B cell lymphoma; gynecological cancer, such as ovarian cancer; breast cancer; liver cancer; lung cancer; or blood cancer, such as myeloma or leukemia, for example, multiple myeloma, ALL, or AML).

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoma (includes indolent and high grade forms; Hodgkin's lymphoma; and non-Hodgkin's lymphoma, such as diffuse large B-cell, follicular, chronic lymphocytic, small lymphocytic, mantle cell, Burkitt's, cutaneous T-cell, AIDS-related, or central nervous system lymphoma), pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma, and retinoblastoma).

In particular examples, hematological malignancies that can be inhibited or treated by the methods disclosed herein include but are not limited to multiple myeloma, chronic lymphocytic leukemia, acute lymphocytic leukemia, acute myeloid leukemia, chronic myelogenous leukemia, pro-lymphocytic/myelocytic leukemia, plasma cell leukemia, NK cell leukemia, Waldenstrom macroglobulinemia, Hodgkin's lymphoma, and non-Hodgkin's lymphoma (indolent and high grade forms; includes diffuse large B-cell, follicular, chronic lymphocytic, small lymphocytic, mantle cell, Burkitt's, cutaneous T-cell, AIDS-related, or central nervous system lymphoma). In additional particular examples, solid tumors that can be treated or inhibited by the methods disclosed herein include lung carcinoma, prostate cancer, pancreatic cancer (for example, insulinoma), breast cancer, colorectal adenocarcinoma or squamous cell carcinoma, neuroblastoma, testicular cancer (such as seminoma), and ovarian cancer. In specific, non-limiting examples, the subject has chronic myelogenous leukemia, acute monocytic leukemia, or non-Hodgkin's lymphoma (indolent and high grade forms; includes diffuse large B-cell, follicular, chronic lymphocytic, small lymphocytic, mantle cell, Burkitt's, cutaneous T-cell, AIDS-related, or central nervous system lymphoma). One of ordinary skill in the art can select NK cells, T cells, macrophages, or NKT cells (such as CAR-NK, CAR-T, CAR-M, or CAR-NKT cells) expressing an appropriate CAR for treating a subject with particular tumors or other disorders.

In some examples, the subject (such as a subject with a cancer) is also administered one or more chemotherapeutic agents and/or radiation therapy. Such agents include alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine); antimetabolites such as folic acid analogs (such as methotrexate), pyrimidine analogs (such as 5-FU or cytarabine), and purine analogs, such as mercaptopurine or thioguanine; or natural products, for example vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Additional agents include platinum coordination complexes (such as cis-diamine-dichloroplatinum II, also known as cisplatin), substituted ureas (such as hydroxyurea), methyl hydrazine derivatives (such as procarbazine), and adrenocrotical suppressants (such as mitotane and aminoglutethimide); hormones and antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of the most commonly used chemotherapy drugs include adriamycin, melphalan (Alkeran®) Ara-C(cytarabine), carmustine, busulfan, lomustine, carboplatinum, cisplatinum, cyclophosphamide (Cytoxan®), daunorubicin, dacarbazine, 5-fluorouracil, fludarabine, hydroxyurea, idarubicin, ifosfamide, methotrexate, mithramycin, mitomycin, mitoxantrone, nitrogen mustard, paclitaxel (or other taxanes, such as docetaxel), vinblastine, vincristine, VP-16, while newer drugs include gemcitabine (Gemzar®), trastuzumab (Herceptin®), irinotecan (CPT-11), leustatin, navelbine, rituximab (Rituxan®) imatinib (STI-571), Topotecan (Hycamtin®), capecitabine, ibritumomab (Zevalin®), and calcitriol.

In some examples, the methods include treating or inhibiting a blood cancer (including indolent and high-grade forms; such as myeloma, such as multiple myeloma; lymphoma, such as Hodgkin's or non-Hodgkin's lymphoma, for example, diffuse large B-cell, follicular, chronic lymphocytic, small lymphocytic, mantle cell, Burkitt's, cutaneous T-cell, AIDS-related, or central nervous system lymphoma; or leukemia, such as acute lymphocytic leukemia (ALL) or acute myeloid leukemia (AML)). For example, the methods can include selecting a subject with a blood cancer. The methods can also include administering any of the NK cells, T cells, macrophages, or NKT cells disclosed herein, such as the CAR-NK, CAR-T, CAR-M, or CAR-NKT cells, to the subject using any effective administration method, thereby treating the blood cancer. For example, CD19 CAR-modified NK cells expanded using the disclosed isolated exosomes can be administered to the subject using any effective administration method. In some examples, exosomes isolated from a supernatant of a culture of 721.221-mIL-21 cells or 721.221-mIL21-B7H6 cells further modified to include IL-15Rα can be used to expand the population of CAR-NK cells (such as autologous CAR-NK cells), such as CD19 CAR-NK cells (such as autologous CD19 CAR-NK cells), administered to the subject.

In specific, non-limiting examples, the methods include treating or inhibiting leukemia (such as acute lymphocytic leukemia (ALL) or acute myeloid leukemia (AML)). For example, the methods can include selecting a subject with leukemia. The methods can also include administering any of the CAR-modified lymphocytes expanded using the methods disclosed herein, thereby treating the leukemia, for example, CD19 CAR-NK cells produced using the disclosed exosomes. In some examples, exosomes isolated from a supernatant of a culture of 721.221-mIL-21 cells or 721.221-mIL21-B7H6 cells further modified to include IL-15Rα can be used to expand the population of CAR-NK cells (such as autologous CAR-NK cells), such as CD19 CAR-NK cells (such as autologous CD19 CAR-NK cells), administered to the subject having the leukemia, thereby treating the leukemia.

In some examples, the methods include treating or inhibiting solid tumors (such as indolent and high-grade forms, sarcomas, carcinomas, and lymphomas (such as Hodgkin's or non-Hodgkin's)). For example, the methods can include selecting a subject with a solid tumor. The methods can also include administering any of the NK cells, T cells, macrophages, or NKT cells (such as the CAR-NK, CAR-T, CAR-M, or CAR-NKT cells) expanded using the methods disclosed herein to the subject, thereby treating the solid tumor.

In specific, non-limiting examples, the methods include treating or inhibiting lymphoma (such as indolent and high-grade forms, Hodgkin's lymphoma, and non-Hodgkin's lymphoma (such as diffuse large B-cell, follicular, chronic lymphocytic, small lymphocytic, mantle cell, Burkitt's, cutaneous T-cell, AIDS-related, or central nervous system lymphoma)). For example, the methods can include selecting a subject with lymphoma. The methods can also include administering any of the NK cells, T cells, macrophages, or NKT cells (such as the CAR-NK, CAR-T, CAR-M, or CAR-NKT cells) expanded using the methods disclosed herein to the subject, thereby treating the lymphoma. In some examples, exosomes isolated from a supernatant of a culture of 721.221-mIL-21 cells or 721.221-mIL21-B7H6 cells further modified to include IL-15Rα can be used to expand the population of CAR-NK cells (such as autologous CAR-NK cells), such as CD19 CAR-NK cells (such as autologous CD19 CAR-NK cells), administered to the subject having the lymphoma, thereby treating the lymphoma.

In some examples, the methods include treating or inhibiting an immune disease or disorder. The immune disease or disorder can be any type of immune system condition, such as a cytokine storm, an immune system disorder (e.g., an inflammatory or autoimmune disorder) or can be immune system conditions associated with another condition and/or disease (e.g., HIV infection or exposure to microgravity). In some non-limiting examples, the immune system disease or disorder is an inflammatory disorder. In specific embodiments, the inflammatory disorder can be rheumatoid arthritis, chronic obstructive pulmonary lung disease, inflammatory bowel disease, or systemic lupus erythematosus. In other examples, the immune system disease or disorder is an autoimmune disorder. In certain embodiments, the autoimmune disorder is type I diabetes, multiple sclerosis, lupus erythematosus, myasthenia gravis, ankylosing spondylitis, celiac disease, Crohn's disease, Graves' disease, Hashimoto's thyroiditis, transplant rejection, or autoimmune uveitis. Modified or unmodified NK cells, T cells, macrophages, or NKT cells expanded using the methods disclosed herein can be used. In specific examples, modified (e.g., CAR-modified) NK cells, T cells, macrophages, or NKT cells can be used, for example, to treat or inhibit rheumatoid arthritis, Crohn's disease, or transplant rejection.

In some examples, the subject (e.g., a subject with an immune disease or disorder, such as an autoimmune disease, transplant rejection, or inflammatory disease) is also administered one or more immunomodulatory therapies (e.g., immunomodulatory biologics, such as muromonab, ipilimumab, abatacept, belatacept, tremelimumab, BMS-936558, CT-011, MK-3475, AMP224, BMS-936559, MPDL3280A, MEDI4736, MGA271, IMP321, BMS-663513, PF-05082566, CDX-1127, anti-OX40, huMAb, OX40L, and TRX518, e.g., Yao et al., Nat Rev Drug Discov, 12(2): 130-146, 2013, and Kamphorst et al., Vaccine, 33(0 2): B21-B28, 2015, both of which are incorporated herein by reference in their entireties; modulatory cytokines, such as IL-7; mTOR modulatory agents, such as rapamycin; antimicrobial therapy, such as vaccination, antifungals, and/or antibiotics), anti-inflammatory agents (NSAIDS; antileukotrines; immune selective anti-inflammatory derivatives, ImSAIDs; bioactive compounds with anti-inflammatory activities, such as plumbagin and plumericin; and/or steroids), disease-modifying antirheumatic drugs (DMARDs, such as methotrexate, sulfasalazine, leflunomide, hydroxychloroquine, tofacitinib, infliximab, etanercept, adalimumab, certolizumab, golimumab, tocilizumab, anakinra, abatacept, and/or rituximab), antimalarial drugs (e.g., chloroquine and hydroxychloroquine), medical procedures (including surgery and stem cell transplantation); immunosuppressive agents (e.g., for preventing rejection of transplanted organs or tissues, treating autoimmune diseases, and/or inflammatory diseases; e.g., glucocorticoids, such as prednisone, dexamethasone, and hydrocortisone; cytostatics, such as alkylating agents and antimetabolites; antibodies, such as Atgam, thymoglobuline, and T-cell receptor- and IL-2 receptor-directed antibodies; immunophilin-targeting agents, such as cyclosporin, tacrolimus, sirolimus, and everolimus; interferons (IFNs), such as IFNλ and IFNβ; opioids; TNF binding proteins, such as infliximab, etanercept, and adalimumab; mycophenolate; and small biological agents, such as fingolimod and myriocin), immune tolerance therapy (e.g., for treating subjects at risk for tissue or organ transplantation rejection, subjects with allergies, and/or subjects with autoimmune disease; e.g., T or B cell-targeting or T or B cell-suppressing drugs, such as CAMPATH-1H, calcineurin inhibitors, rituximab, epratuzumab, belimumab, and atacicept; anti-cluster of differentiation (CD)3 antibodies; abatacept; induction of hematopoietic chimerism, such as mixed hematopoietic chimerism, in which the bone marrow of an organ or a tissue recipient is replaced with the donor's bone marrow or a mixture of the donor and recipient bone marrow to reduce organ or tissue transplant rejection; antigen desensitization; see Nepom et al., Immunol Rev; 241(1): 49-62, 2011, incorporated herein by reference), antihistamines, helminthic therapies (e.g., deliberate infestation of the subject with a helminth or with the ova of a helminth for treating immune disorders).

In some examples, the methods include treating or inhibiting an infectious disease by administering a population of NK cells, T cells, macrophages, or NKT cells expanded using the methods disclosed herein to a subject. In some examples, the infectious disease is selected from among arboviral infections, botulism, brucellosis, candidiasis, campylobacteriosis, chickenpox, chlamydia, cholera, coronovirus infections, staphylococcus infections, coxsackie virus infections, Creutzfeldt-Jakob disease, cryptosporidiosis, cyclospora infection, cytomegalovirus infections, Epstein-Barr virus infection, dengue fever, diphtheria, ear infections, encephalitis, influenza virus infections, parainfluenza virus infections giardiasis, gonorrhea, Haemophilus influenzae infections, hantavirus infections, viral hepatitis, herpes simplex virus infections, HIV/AIDS, helicobacter infection, human papillomavirus (HPV) infections, infectious mononucleosis, legionellosis, leprosy, leptospirosis, listeriosis, lyme disease, lymphocytic choriomeningitis, malaria, measles, marburg hemorrhagic fever, meningitis, monkeypox, mumps, mycobacteria infection, mycoplasma infection, norwalk virus infection, pertussis, pinworm infection, pneumococcal disease, Streptococcus pneumonia infection, Mycoplasma pneumoniae infection, Moraxella catarrhalis infection, Pseudomonas aeruginosa infection, rotavirus infection, psittacosis, rabies, respiratory syncytial virus infection (RSV), ringworm, rocky mountain spotted fever, rubella, salmonellosis, SARS, scabies, sexually transmitted diseases, shigellosis, shingles, sporotrichosis, streptococcal infections, syphilis, tetanus, trichinosis, tuberculosis, tularemia, typhoid fever, viral meningitis, bacterial meningitis, west Nile virus infection, yellow fever, adenovirus-mediated infections and diseases, retrovirus-mediated infectious diseases and yersiniosis zoonoses. For example, the infectious disease can be influenza, parainfluenza, respiratory syncytial virus.

Unmodified or modified (e.g., CAR-modified) NK cells, T cells, macrophages, or NKT cells expanded using the methods disclosed herein can be used to treat or inhibit an infectious disease. In specific examples, CAR-modified NK cells, T cells, macrophages, or NKT cells expanded using the disclosed methods can be used to treat or inhibit HIV, such as using CARs based on HIV antibodies VRC01, 2G12, 2F5, 4E10, 3BNC117, 10-1074, VRC01LS, VRC07-532LS, 3BC176, PG16, NIH45-46G54W, PG9, PG16, PGT145, PGDM1400, PGT121, PGT124, PGT128, PGT135, 8ANC195, 10E8, and/or PD-1. In specific examples, CAR-modified NK or T cells expanded using the disclosed methods can be used to treat or inhibit HBV, such as using CARs targeting HBsAg (e.g., GENBANK® nos. KP972453.1 or KP972454.1) and/or HB1.

In some examples, the subject (e.g., a subject with an infectious disease, such as HIV) is also administered one or more anti-infection agents (e.g., antibodies, antifungals, antivirals, and/or antiparasitics). In specific examples, the infectious disease is HIV, and the subject is also administered antiretroviral agents, such as nucleoside and nucleotide reverse transcriptase inhibitors (nRTI), non-nucleoside reverse transcriptase inhibitors (NNRTI), protease inhibitors, entry inhibitors (or fusion inhibitors), maturation inhibitors, or a broad-spectrum inhibitors, such as natural antivirals. Other exemplary agents include lopinavir, ritonavir, zidovudine, lamivudine, tenofovir, emtricitabine, and efavirenz.

### EXAMPLES

The following examples are provided to illustrate certain particular features and/or embodiments. These examples should not be construed to limit the disclosure to the particular features or embodiments described.

The Examples provided herein describe a non-feeder cell (NFC) (or feeder cell-free/less) system to expand NK and CAR-NK cells in vitro. Primary NK cells expanded using the NFC expansion system have been evaluated for expansion capability, surface markers, and cytotoxic functions. ‘Off-the-shelf’ NK cells expanded using the described NFC expansion system will support clinical use of NK immunotherapy.

The NFC expansion system developed herein resulted in a dramatically increased expansion rate in both the PBMC-expanded and pNK cell-expanded NK cells as compared to the expansion rate of PBMCs and pNK cells treated with cytokines only. Expansion of NK cells using the NFC system was slightly lower than that of FC expansion system. However, compared to the FC expansion system, the NFC expansion system prolonged NK cell survival in vitro, making it more useful for clinical applications. Further, NK cells expanded from both PBMCs and pNK cells using the NFC system showed similar cytotoxicities towards K562 target cells as compared to FC-expanded PBNK cells.

Without being limited to a particular mechanism of action, it is hypothesized that IL-21 and other stimulatory molecules (e.g., CD28 molecules) in the disclosed exosomes can stimulate NK cell proliferation. Exosome surface proteins may bind with toll-like or NKG2D receptors on NK cells to facilitate NK cell expansion. Additionally, cytokines produced by monocytes or T cells from PBMCs stimulated by the disclosed exosomes, together with the direct effects of exosomes on NK cells, may further stimulate NK cell expansion.

In addition, the Examples describe 721.221 cells that express both mIL21 and B7-H6. These cells are shown to result in 3-5-fold faster expansion of NK cells than 721.221 cells expressing mIL21 alone, when used as a feeder cell system.

In summary, the Examples herein demonstrate that the disclosed exosome technology can be used to expand populations of immune cells, such as NK or CAR-NK cells, in the absence of feeder cells.

### Example 1

### Materials and Methods

### Antibodies and Reagents:

PE and APC anti-human CD3 antibody (clone OKT3, BioLegend), FITC, BV605, PE/Cy7, and BV 510 anti-human CD56 antibody (clone HCD56, BioLegend), PE anti-human CD69 antibody (clone FN50, BioLegend), PE/Cy7 anti-human CD8a antibody (clone HIT8a, BioLegend), AF647 anti-human IL-21 antibody (clone 3A3-N2, BioLegend), BV 421 anti-human CD335 (NKp46) antibody (clone 9E2, BioLegend), PE/Cy7 anti-human CD158b (KIR2DL2/L3, BioLegend) antibody (clone DX27, BioLegend), PE/Cy7 anti-human CD244 (2B4) antibody (clone C1.7, BioLegend), APC anti-human CD366 (Tim-3) antibody (clone F38-2E2), PerCP/Cy5.5 anti-human TIGIT (VSTM3) antibody (clone A15153G), FITC anti-human CD223 (LAG-3) antibody (clone 11C3C65, BioLegend), and PerCP/Cy5.5 anti-human CD94 (clone DX22, BioLegend) were purchased from BioLegend (San Diego, CA, USA). APC anti-human CD16 antibody (clone B73.1, BD Biosciences), FITC anti-human CD3 antibody (clone UCHT1, BD Biosciences), BV711 anti-human CD314 (NKG2D) antibody (clone 1D11, BD Biosciences), and FITC anti-human CD107a antibody (clone H4A3, BD Biosciences) were purchased from BD Biosciences (San Jose, CA, USA). FITC anti-human KIR/CD158 antibody (clone 180704, R&D Systems), PE anti-human KIR2DL1/KIR2DS5 antibody (clone 143211, R&D Systems), APC anti-human KIR3DL1 antibody (clone DX9, R&D Systems), APC anti-human NKG2A/CD159a antibody (clone 131411, R&D Systems), and PE anti-human NKG2C/CD159c antibody (clone 134591, R&D Systems) were purchased from R&D Systems. AF647 Goat anti-human IgG(H+L) F(ab′)₂ fragment antibody was purchased from Jackson ImmunoResearch (West Grove, PA, USA).

### Cell Lines:

The 721.221 cell line was a gift from Dr. Eric 0. Long (National Health of Allergy and Infectious Diseases in National Health of Institutes). 293T and K562 cell lines were purchased from the American Type Culture Collection (ATCC). To establish K562-mIL-21 and 721.221-mIL-21 cells, K562 and 721.221 cells were transduced with IL-21 retrovirus, respectively, and then membrane IL-21 positive cells were sorted using FACS Aria II cell sorter (BD Biosciences) using AF647 mouse IgG1 anti-human IL-21 (clone 3A3-N2) as previously described (Xiong et al., Mol Ther. 26(4):963-975, 2018). 721.221-mIL-21 cells were cultured in RPMI-1640 (Corning) supplemented with 10% (v/v) fetal bovine serum (FBS) and 100 U/mL penicillin-streptomycin (Corning) at 37° C. with 5% (v/v) CO₂. For NK cell expansion, 721.221-mIL-21 cells were irradiated at a dose of 10,000 Rad, washed with PBS, and then used as feeder cells. 293T cells were cultured in DMEM (Corning) supplemented with 10% (v/v) fetal bovine serum (FBS) and 100 U/mL penicillin-streptomycin (Corning) at 37° C. with 5% (v/v) CO₂.

### Exosome Isolation and Mass Spectrometry:

721.221-mIL-21 cells (10⁶/mL in 50 mL) were cultured with 1 μg/mL PMA, 1 μg/mL ionomycin, and 1 μg/mL R848 for 48-72 hrs. All culture media (50 mL) for isolating exosomes were serially centrifuged at 300×g for 5 minutes to remove live cells, then at 1,200×g for 20 min first to remove dead cells, and then at 10,000×g to remove debris and apoptotic bodies. To obtain highly pure concentrated exosomes, these exosome samples were further ultracentrifuged at 160,000×g for 60 min and then washed twice with PBS. The final exosome pellet was resuspended in 1 ml 1×PBS. Exosomes went through 0.22-μm filtration before used for cell culture.

Exosomes were lysed with cell lysis buffer and protein concentrations were determined. Samples were run on 10% SAS-PAGE gels, and gels were then fixed in 50% methanol with 10% acetic acid buffer for 1 hour before Coomassie blue staining. The stained gels were then sent to the Proteomics core facility (Rutgers-Cancer Center) for mass spectrometry analysis.

### Primary NK Cell Expansion:

PBMCs were isolated from buffy coats (New York Blood Center) using Lymphocyte Separation Medium (Corning). For NK cell expansion, 5×10⁶ PBMCs were cultured with cytokines only (200 U/ml IL-2 and 5 ng/ml IL-15; baseline control), exosomes (250 μL; the non-feeder cell expansion system), or 1×10⁷ irradiated feeder cells in 35 ml RPMI-1640 media with 10% Fetal Bovine Serum (Corning), 2 mM L-Glutamine (Corning), 100 U/ml penicillin-streptomycin (Corning), 200 U/ml IL-2 (PeproTech), and 5 ng/ml IL-15 (PeproTech) in G-Rex 6 multi-well cell culture plates (Wilson Wolf). Media were changed every 3-4 days and 2×10⁷ cells were kept in each well for continued culture. Total cell numbers were counted using Trypan Blue and an automated cell counter (Nexcelom, MA). To determine the percentage of NK cells, cells were stained for CD3 and CD56 for flow cytometry analysis.

Transduction of Expanded NK Cells with CD19-CAR:

To produce CD19-CAR retrovirus, 293T cells were transfected with a combination of plasmids containing CD19-CAR in SFG backbone, RDF, and PegPam3, as previously described (Xiong et al., Mol Ther. 26(4):963-975, 2018). NK cells were harvested on day 7 of expansion and transduced with CD19-CAR retrovirus in plates coated with RetroNection (Clontech). Two days later, cells were transferred to G-Rex 6 multi-well cell culture plates and maintained in 35 ml complete RPMI-1640 media with 200 U/ml IL-2 (PeproTech) and 5 ng/ml IL-15 (PeproTech). Media were changed every 3-4 days and 2×10⁷ cells were kept in each well for continued culture. Total cell numbers were counted using Trypan Blue and an automated cell counter (Nexcelom, MA). To determine the percentage of NK cells and expression of CAR, cells were stained for CD3, CD56, and anti-human IgG(H+L) F(ab′)₂ fragment for flow cytometry analysis.

### Flow Cytometry Analysis:

PBMCs and expanded NK cells were stained with fluorescence-conjugated antibodies in FACS staining buffer (PBS with 1% FBS) on ice for 30 minutes, washed with PBS, and analyzed on a FACS LSRII or an LSR Fortessa flow cytometer (BD). PMT voltages were adjusted and compensation values were calculated before data collection. Data were acquired using FACS Diva software (BD) and analyzed using FlowJo software (BD).

### Flow Cytometry-based NK Cytotoxicity Assays:

K562 and 721.221 cells were used as target cells to determine NK cell cytotoxicity. Target cells were harvested and stained with 5 μM CellTrace CFSE (Invitrogen) in PBS for 20 minutes. The staining was stopped by adding complete RPMI-1640 media and then cells were washed twice using PBS. Expanded NK cells were harvested and cocultured with 2×10⁵ CFSE-labeled target cells at 5 different E:T ratios (4:1, 2:1, 1:1, 0.5:1, and 0.25:1) in V-bottomed 96-well plates in complete RPMI-1640 media. After 4 hrs of incubation at 37° C. in the presence of 5% CO₂, cells were stained with 7-AAD (eBioscience) and then analyzed using flow cytometry. Target cells (CFSE⁺) were gated, and the percent of 7-AAD⁺ cells was used to calculate NK cell cytotoxicity using the following equation: (Experimental—Spontaneous dead cells)/(100−Spontaneous dead cells)×100%.

### CD107a Degranulation Assay:

The CD107a degranulation assay was described previously (Zheng et al., J Allergy Clin Immunol. 135(5):1293-302, 2015). Briefly, expanded NK cells (5×10⁴) were incubated with 1.5×10⁵ K562 cells in V-bottomed 96-well plates in complete RPMI-1640 media at 37° C. for 2 hrs. The cells were harvested, washed, and stained for CD3, CD56, and CD107a with GolgiStop for 30 minutes, and analyzed using flow cytometry.

### ⁵¹Cr Release Assay:

To evaluate the cytotoxic activity of PBNK cells, the standard 4-hr ⁵¹Cr release assay was used. Briefly, target K562 cells were labeled with ⁵¹Cr at 37° C. for 2 hrs and then resuspended at 2×10⁵/mL in RPMI1640 medium with 10% FBS. Then, 1×10⁴ target cells were incubated with serially diluted PBNK cells at 37° C. for 4 hrs. After centrifugation, the supernatants were collected and the released ⁵¹Cr was measured with a gamma counter (Wallac, Turku, Finland). The cytotoxicity (as a percentage) was calculated as follows: [(sample−spontaneous release)/(maximum release−spontaneous release)]×100.

### Statistical Analysis:

Data were represented as means±SEM. Statistical significance was determined using a two-tailed unpaired Student t test, a two-tailed paired Student t test, or a two-way ANOVA, where indicated. P<0.05 was considered statistically significant.

### Example 2

### Generation of 721.221-mIL-21 Feeder Cells

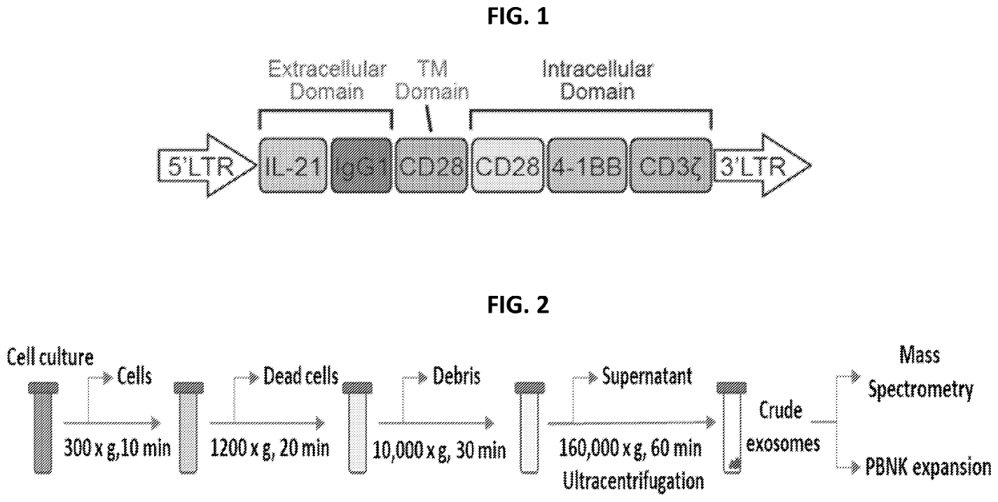

Previous work by the inventors showed that the mIL-21-encoding SFG retroviral vector used herein (FIG. 1, SEQ ID NOs: 3 and 10) significantly improved the transduction efficiency of hematopoietic cells (International Patent Application Publication WO 2020/172328). Transduction of K562 and 721.221 cells with the SFG vector resulted in high expression of mIL-21 on both cell lines. The extracellular and intracellular domains of SFG (containing the IgG1 CH2-CH3 domain, CD28 transmembrane and intracellular domains, 4-1BBintracellular domain, and CD3ζ intracellular domain) maintained high stability of the mIL-21 motif, which is desirable for industrial manufacturing. Additionally, the intracellular domains served as metabolism regulatory factors in 721.221-mIL-21 cells to induce NK cell proliferation and expansion.

### Example 3

### Generation of Exosomes from 721.221-mIL-21 Feeder Cells

721.221-mIL-21 cells were stimulated with a combination of various TLR agonists. TLR agonists used herein included LPS, CpG-oligodeoxynucleotides (CpG-Oligo), R848, PHA, PMA, IL-2, ionomycin, or poly(I:C), and combinations thereof. Working concentration ranges for the TLR agonists used herein are shown in Table 1.

TABLE 1
TLR agonists used to stimulate 721.221-mIL-21 cells.

| Name | Working Concentration | |
|---|---|---|
| CpG-Oligo | 0.24-2.4 | μg/mL |
| R848 | 0.1-1 | μg/mL |
| Human IL-2 | 1-10 | ng/mL |
| PHA | 2.5-25 | μg/mL |
| PMA | 0.1-1 μg/mL = 0.162-1.62 μM | |
| Ionomycin | 0.1-1 μg/mL = 0.134-1.34 μM | |
| LPS | 0.5-5 | μg/mL |
| Poly(I:C) | 0.1-1 | μg/mL |

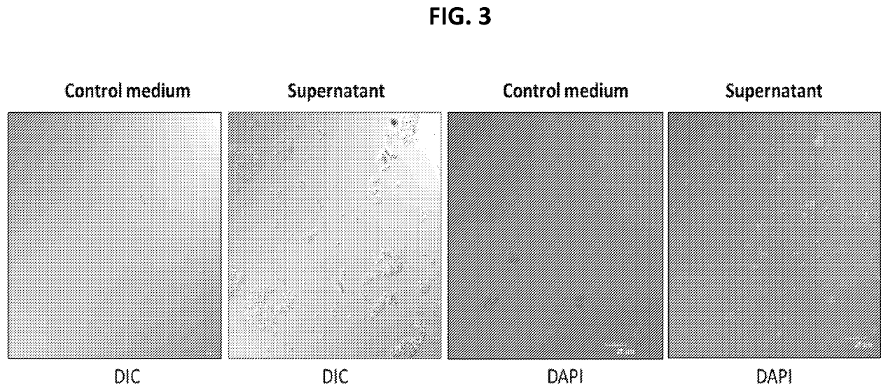

Exosomes were isolated using serial ultracentrifugation (FIG. 2), as described in Example 1 and below. Previous studies showed that IL-21 plays a role in NK cell proliferation and promotes the expansion of memory-like NK cells (Ojo et al., Sci Rep. 9(1):14916, 2019; Denman et al., PLoS One. 7(1):e30264, 2012; Venkatasubramanian et al., Mucosal Immunol. 10(4):1031-1042, 2017). Moreover, clinical trials showed that NK cells and CAR-NK cells expanded with K562 cells expressing mIL-21 can be safely infused (Liu et al., N Engl J Med. 382(6):545-553, 2020; Ciurea et al., Blood. 130(16):1857-1868, 2017). To expand human NK and CAR-NK cells, a previously developed an artificial antigen presenting cell line using 721.221 cells expressing a membrane form of IL-21 was utilized (Yang, et al., Mol Ther Methods Clin Dev. 18:428-445, 2020). Based on this feeder cell (FC) expansion technology, exosomes were isolated herein from 721.221-mIL-21 cells stimulated with the combination of TLR agonists for 72 hours (FIG. 3). 721.221-mIL-21 cell culture supernatants were centrifuged at 300×g for 5 min to remove live cells, were further centrifuged at 1200×g for 20 min to remove dead cells, and were further centrifuged at 10,000×g for 30 min to eliminate debris. The supernatants were then ultracentrifuged at 160,000×g for 60 min at 4° C. to pellet exosomes. The exosome pellets were washed twice in a large volume of PBS and were recovered by centrifugation at 100,000×g for 1 hour. After exosome quantification, PBMCs or purified NK cells were mixed with different concentrations of exosomes to optimize the NK cell expansion rate.

### Example 4

### Characteristics of Exosomes Generated from 721.221-mIL-21 Feeder Cells

Exosomes isolated from 721.221-mIL-21 cells were characterized using confocal microscopy. Previous studies showed that exosomes contain DNA and RNA, as well as lipids, metabolites, and cytoplasmic and membrane proteins, from cancer cells (Perocheau et al., Br J Pharmacol, 178(12): 2375-2392, 2021).

Thus, DAPI was used to stain the exosomes. Media without 721.221-mIL-21 cells was used as a control. Confocal microscopy results showed positive nuclear staining, indicating the presence of exosomes (FIG. 3).

Exosomes were further characterized using mass spectrometry. Proteins were isolated from exosomes using cell lysis buffer and protein concentrations were determined. Samples were run on 10% SAS-PAGE gels and fixed for Coomassie blue staining. Stained gels were sent to the Proteomics core facility (Rutgers-Cancer Center) for mass spectrometry analysis using SCIEX OS software. Proteins with higher abundances in exosomes are listed in Table 2.

TABLE 2
Human proteins with high abundances in exosomes as determined using label-free quantification.

| | | | | | | | | Ratio |
|---|---|---|---|---|---|---|---|---|
| | | # Unique | | MW | Calc. | Abund | Abund | (Exo/ |
| Description | Acc. No. | Peptides | # AA | (kDa) | PI | (Ctrl) | (Exo) | Ctrl) |
| Structural maintenance of chromosomes | O95347 | 3 | 1197 | 135.6 | 8.43 | 0 | 300 | 300.0 |
| protein 2 | | | | | | | | |
| Ufm1-specific protease 2 (Fragment) | H0Y9B0 | 2 | 197 | 22.4 | 5.68 | 0 | 300 | 300.0 |
| Kinesin-1 heavy chain | P33176 | 2 | 963 | 109.6 | 6.51 | 0 | 300 | 300.0 |
| Tetraspanin (Fragment) | E9PRJ8 | 2 | 209 | 22.5 | 6.15 | 0 | 300 | 300.0 |
| Nuclear receptor-binding protein | Q9UHY1 | 2 | 535 | 59.8 | 5.08 | 0 | 300 | 300.0 |
| Protein RER1 | Q5T091 | 2 | 187 | 21 | 8.66 | 0 | 300 | 300.0 |
| Enolase-phosphatase E1 | Q9UHY7 | 2 | 261 | 28.9 | 4.78 | 0 | 300 | 300.0 |
| Proteasome assembly chaperone 1 | O95456 | 2 | 288 | 32.8 | 7.17 | 0 | 300 | 300.0 |
| Ribosomal biogenesis protein LAS1L | Q9Y4W2 | 2 | 734 | 83 | 4.73 | 0 | 268.7 | 300.0 |
| WD repeat-containing protein 18 | Q9BV38 | 2 | 432 | 47.4 | 6.7 | 0 | 232.5 | 300.0 |
| Cell cycle control protein | A0A669 | 2 | 289 | 32.5 | 9.26 | 0 | 217.9 | 300.0 |
| | KBF5 | | | | | | | |
| BH3-interacting domain death agonist | P55957 | 1 | 195 | 22 | 5.44 | 0 | 300 | 300.0 |
| Mitochondrial assembly of ribosomal | Q96EH3 | 1 | 234 | 26.2 | 5.49 | 0 | 300 | 300.0 |
| large subunit protein 1 | | | | | | | | |
| Methylsterol monooxygenase 1 | Q15800 | 1 | 293 | 35.2 | 7.23 | 0 | 300 | 300.0 |
| MIT domain-containing protein 1 | A0A1W2 | 1 | 105 | 12 | 8.69 | 0 | 300 | 300.0 |
| | PP14 | | | | | | | |
| G-protein coupled receptor 183 | P32249 | 1 | 361 | 41.2 | 9.11 | 0 | 300 | 300.0 |
| Leukotriene A-4 hydrolase | P09960 | 1 | 611 | 69.2 | 6.18 | 0 | 300 | 300.0 |
| Ribosomal protein 63, mitochondrial | Q9BQC6 | 1 | 102 | 12.3 | 11.44 | 0 | 300 | 300.0 |
| Galactokinase | P51570 | 1 | 392 | 42.2 | 6.46 | 0 | 300 | 300.0 |
| Ferritin heavy chain | P02794 | 1 | 183 | 21.2 | 5.55 | 0 | 300 | 300.0 |
| Pleckstrin homology domain-containing | A8K727 | 1 | 425 | 47.2 | 8.66 | 0 | 300 | 300.0 |
| family A member 2 | | | | | | | | |
| Cyclin-dependent kinase 2 | P24941 | 1 | 298 | 33.9 | 8.68 | 0 | 300 | 300.0 |
| WW domain-binding protein 2 | Q969T9 | 1 | 261 | 28.1 | 5.91 | 0 | 300 | 300.0 |
| 2-methoxy-6-polyprenyl-1,4- | Q5HYK3 | 1 | 327 | 37.1 | 6.95 | 0 | 300 | 300.0 |
| benzoquinol methylase, mitochondrial | | | | | | | | |
| SLAM family member 7 (Fragment) | R4GND0 | 1 | 236 | 25.9 | 8.34 | 0 | 300 | 300.0 |
| Ceruloplasmin | P00450 | 1 | 1065 | 122.1 | 5.72 | 0 | 300 | 300.0 |
| Dihydropyrimidinase-related protein 2 | Q16555 | 1 | 572 | 62.3 | 6.38 | 0 | 300 | 300.0 |
| Deoxycytidine kinase | D6RCP9 | 1 | 199 | 22.9 | 5.12 | 0 | 300 | 300.0 |
| DNA-directed RNA polymerase II | P19387 | 1 | 275 | 31.4 | 4.92 | 0 | 277.6 | 300.0 |
| subunit RPB3 | | | | | | | | |
| Alpha-fetoprotein | J3KMX3 | 1 | 622 | 70.4 | 5.55 | 0 | 265.4 | 300.0 |
| Nucleosome assembly protein 1-like 1 | F8W543 | 1 | 328 | 38.2 | 4.56 | 0 | 263.4 | 300.0 |
| Vesicle-associated membrane protein 5 | O95183 | 1 | 116 | 12.8 | 7.85 | 0 | 260.3 | 300.0 |
| Charged multivesicular body protein | I3L3E4 | 1 | 113 | 12.9 | 4.15 | 0 | 254.9 | 300.0 |
| 6 (Fragment) | | | | | | | | |
| Stromal membrane-associated protein 2 | Q8WU79 | 1 | 429 | 46.8 | 8.87 | 0 | 244.4 | 300.0 |
| Peptidyl-prolyl cis-trans isomerase | Q13526 | 1 | 163 | 18.2 | 8.82 | 0 | 235.4 | 300.0 |
| NIMA-interacting 1 | | | | | | | | |
| Ubiquitin carboxyl-terminal | Q14694 | 1 | 798 | 87.1 | 5.31 | 0 | 234.7 | 300.0 |
| hydrolase 10 | | | | | | | | |
| Peroxisomal biogenesis factor 3 | P56589 | 1 | 373 | 42.1 | 8.15 | 0 | 231.5 | 300.0 |
| Receptor expression-enhancing protein 5 | Q00765 | 1 | 189 | 21.5 | 8.1 | 0 | 228 | 300.0 |
| NADH dehydrogenase [ubiquinone] 1 | Q9NX14 | 1 | 153 | 17.3 | 5.22 | 0 | 224.6 | 300.0 |
| beta subcomplex subunit 11, mitochondrial | | | | | | | | |
| BET1-like protein | E9PHY4 | 1 | 92 | 10.2 | 9.99 | 0 | 220.2 | 300.0 |
| 1-acyl-sn-glycerol-3-phosphate | Q9NUQ2 | 1 | 364 | 42 | 9.1 | 0 | 220.1 | 300.0 |
| acyltransferase epsilon | | | | | | | | |
| Coiled-coil-helix-coiled-coil-helix | Q96BP2 | 1 | 118 | 13.5 | 10.21 | 0 | 217 | 300.0 |
| domain-containing protein 1 | | | | | | | | |
| Inactive C-alpha-formylglycine- | A0A494 | 1 | 305 | 34 | 9.03 | 0 | 189.5 | 300.0 |
| generating enzyme 2 | C1B1 | | | | | | | |

AA: Amino acids; MW: Molecular weight; Abund: Abundance; Ctrl: Control; Exo: Exosomes; PI: Isoelectric point.

### Example 5

### NK Cell Expansion by Exosomes Derived from 721.221-mIL-21 Cells

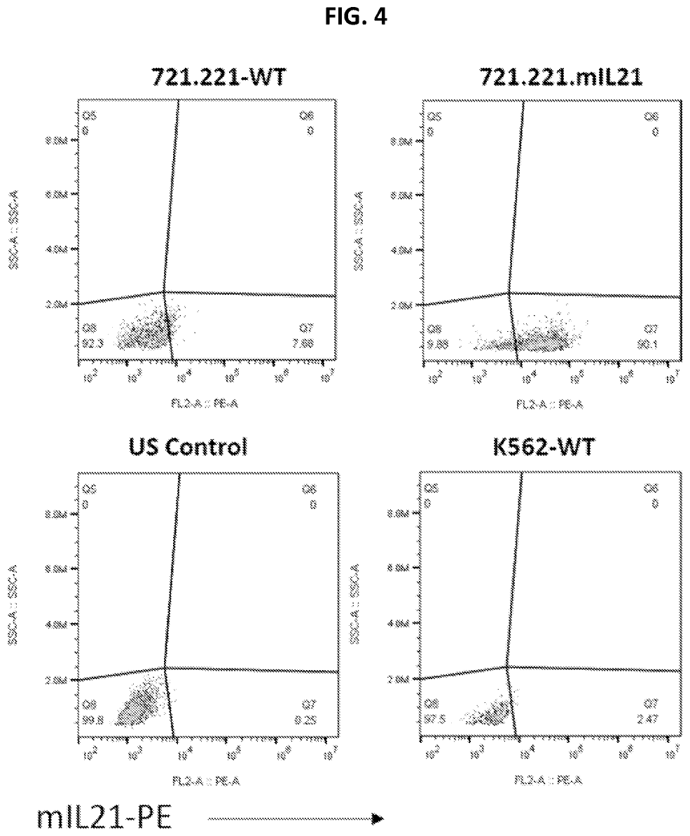

Exosomes isolated from the 721.221-mIL-21 feeder cells were assessed for their ability to expand human primary NK or CAR-NK cells. First, mIL-21 expression was measured in the 721.221-mIL-21 cells before the cells were irradiated and used in the NK expansion experiments (FIG. 4). There was no mIL-21 expression in naïve 721.221 cells (or in naïve K562 cells tested at the same time).

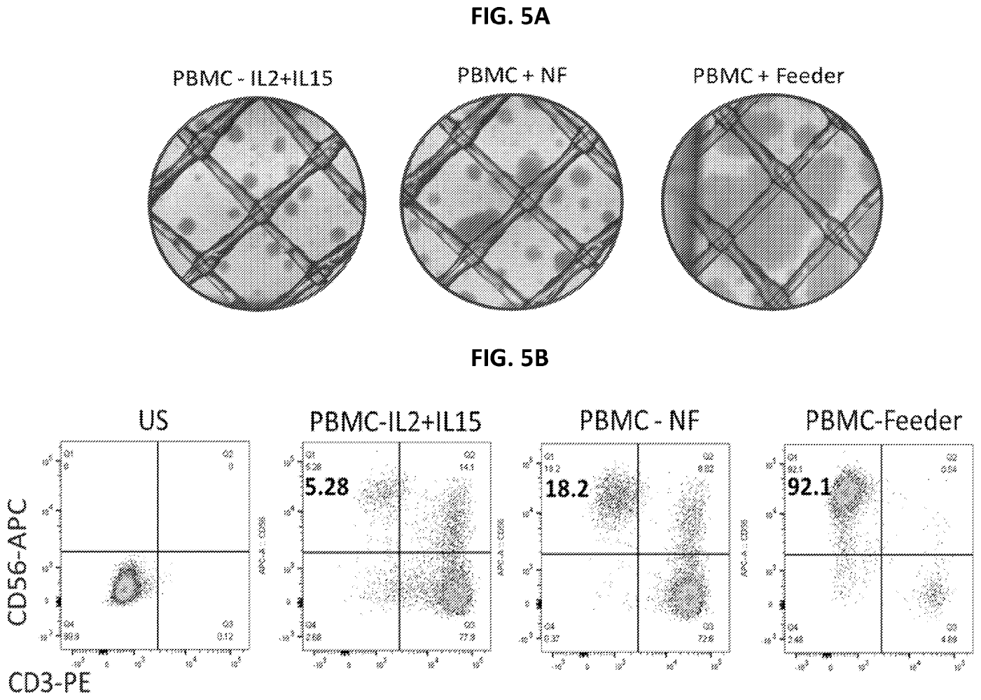

To measure exosome ability to stimulate NK cell expansion from peripheral blood (hereinafter, PBNK cells), PBMCs were isolated from buffy coats of healthy donors and co-cultured with exosomes. PBMCs cultured with 721.221-mIL-21 feeder cells were used as positive controls and PBMCs stimulated with 200 U/mL IL-2 and 5 ng/mL IL-15 only were used as negative controls (FIG. 5). The initial number of PBMCs and proportion of NK cells were 5 million and 5%-10%, respectively. Both the NFC and FC expansion systems resulted in greater NK cell expansion rates compared to cytokines alone. The NFC expansion system resulted in a more than 400-fold (day 21) and 1400-fold (day 28) increase in NK cell numbers, with prolonged NK survival in vitro. Representative NK expansion profiles from cell cultures (FIG. 5A) and CD56 and CD3 flow cytometry analyses for each group expanded from PBMCs (FIG. 5B) are shown.

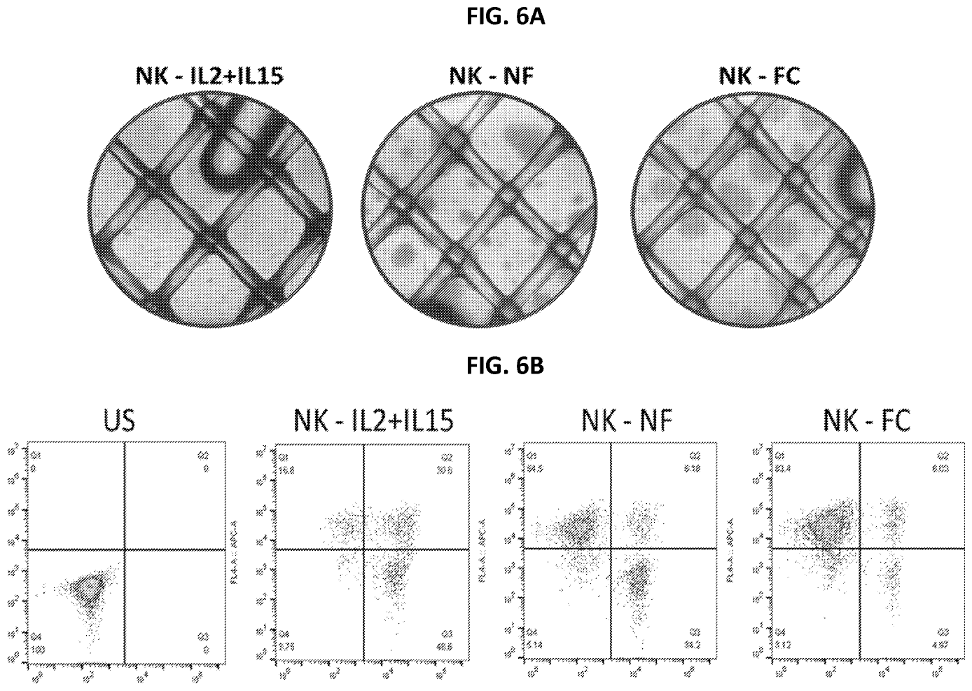

Next, exosomes isolated from feeder cells were assessed for their ability to expand NK cells purified from PBMCs. pNK cell number and purity was compared between three treatment groups: (1) cytokines only (IL-2 and IL-15), (2) exosomes with IL-2 and IL-15 (the NFC expansion system), and (3) 721.221-mIL-21 feeder cells with IL-2 and IL-15. The initial number of pNKs was 5 million. Additional experiments (not shown) used a starting number of 1 million or 3 million pNKs. Both the NFC and FC expansion systems resulted in greater NK cell expansion rates compared to cytokines alone (FIG. 6). The NFC expansion system resulted in a 340-fold (day 21) increase in NK cell numbers. NK cell purities were also much higher for the NFC and FC expansion systems compared to cytokines alone.

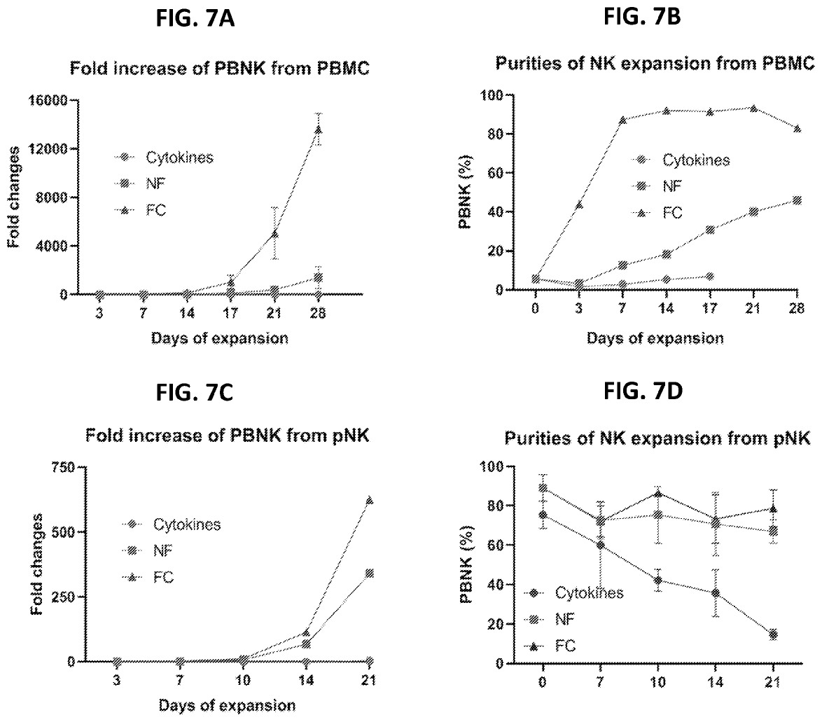

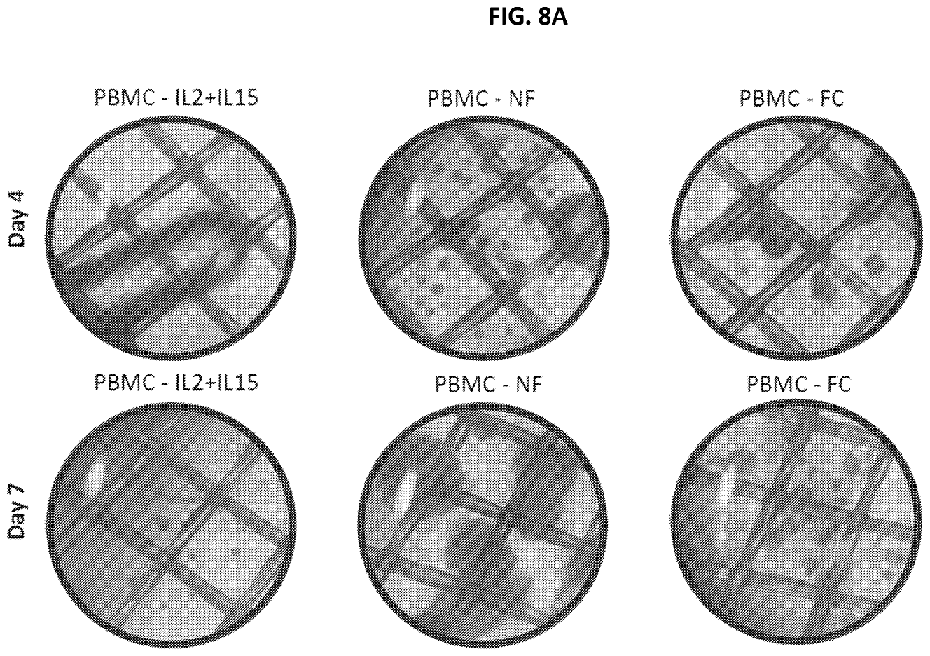

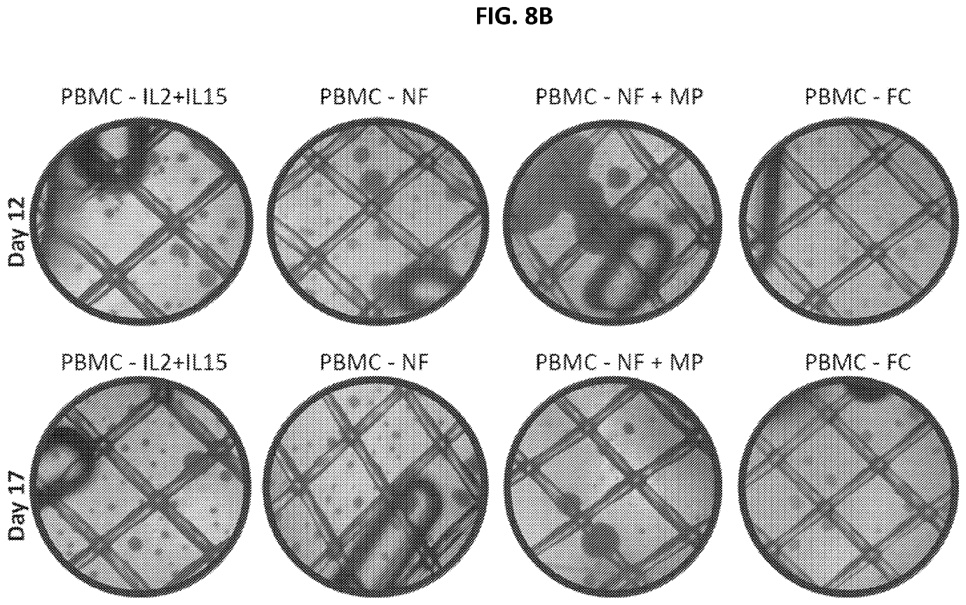

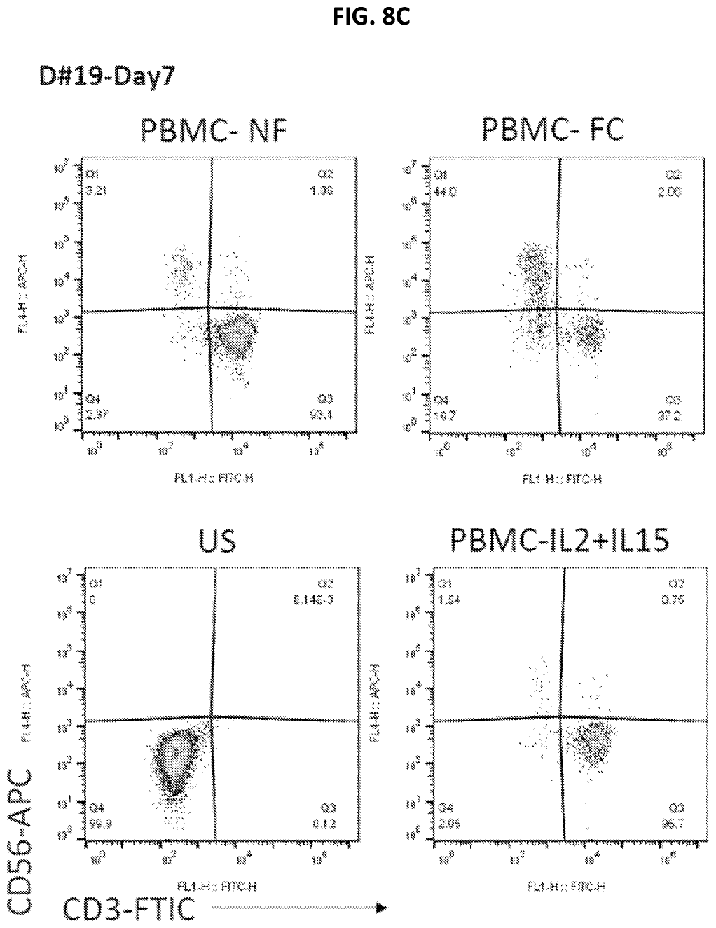

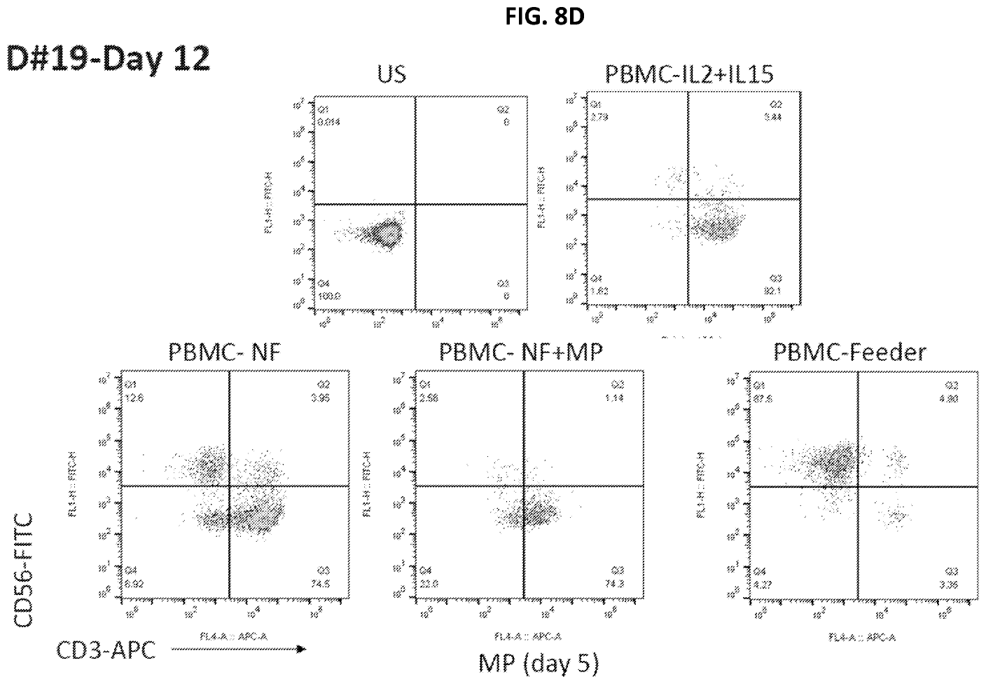

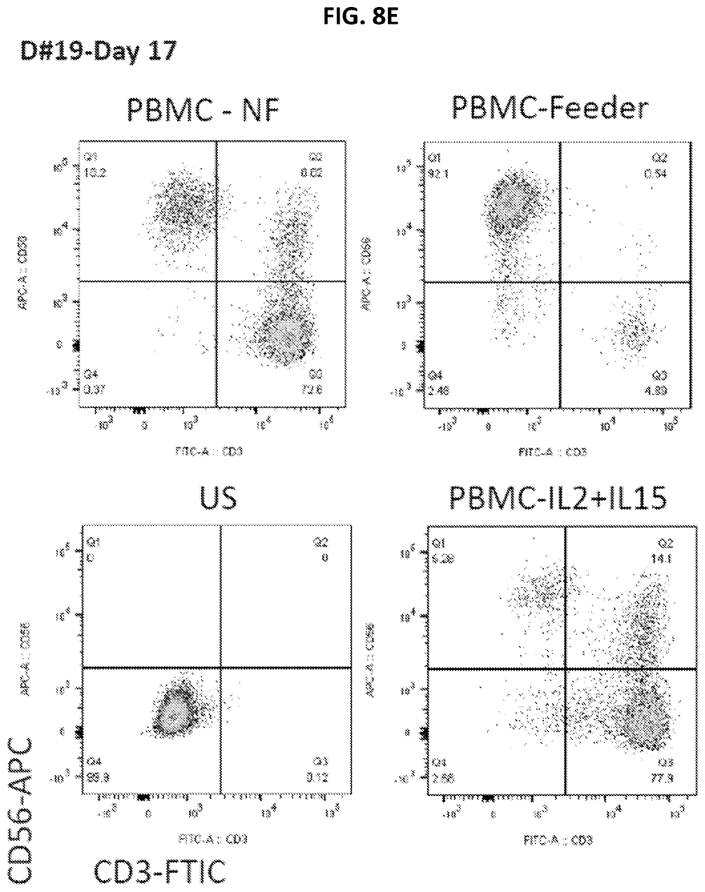

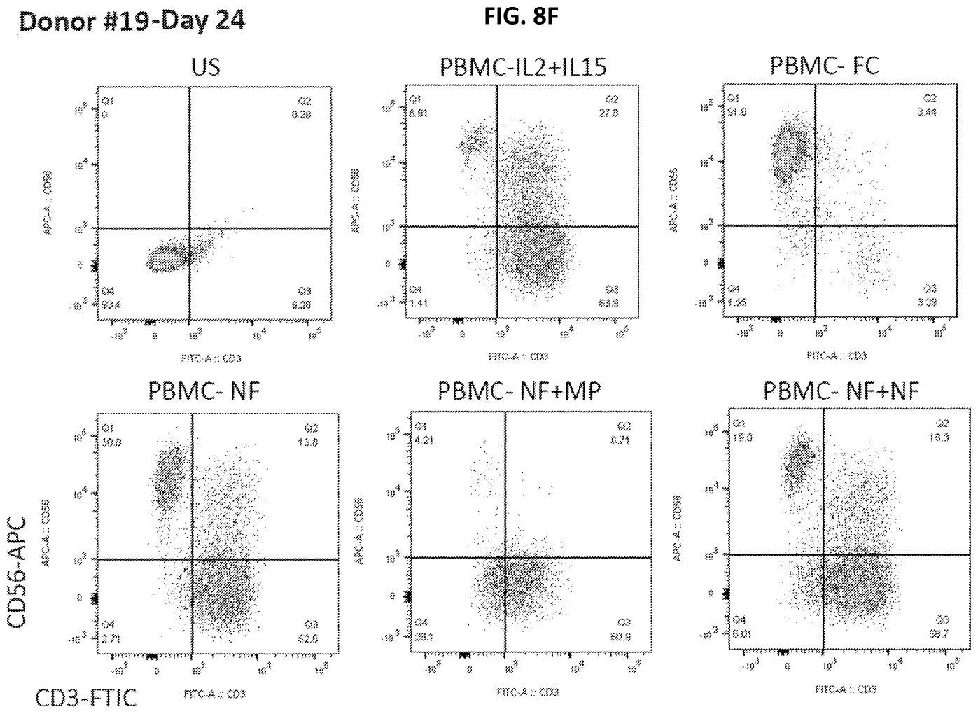

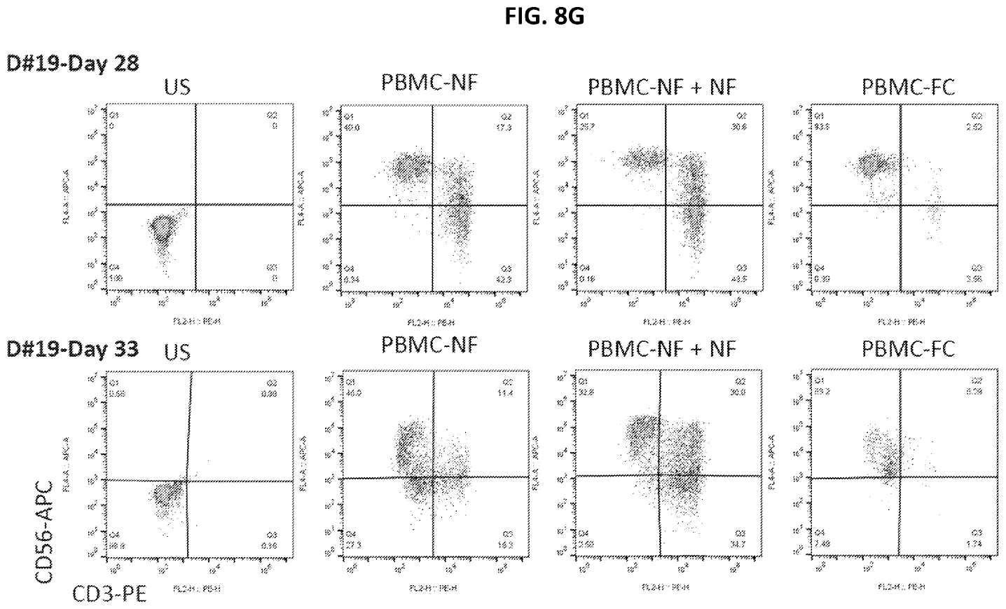

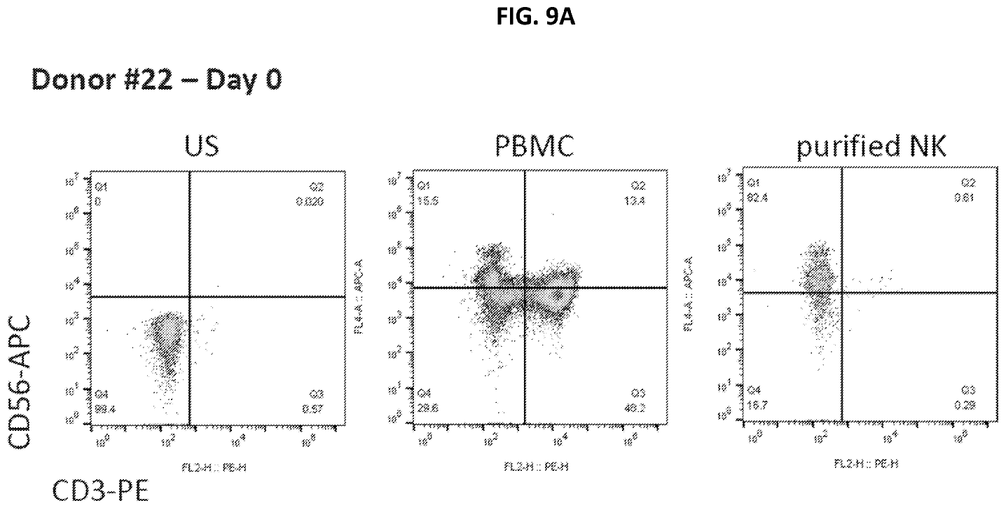

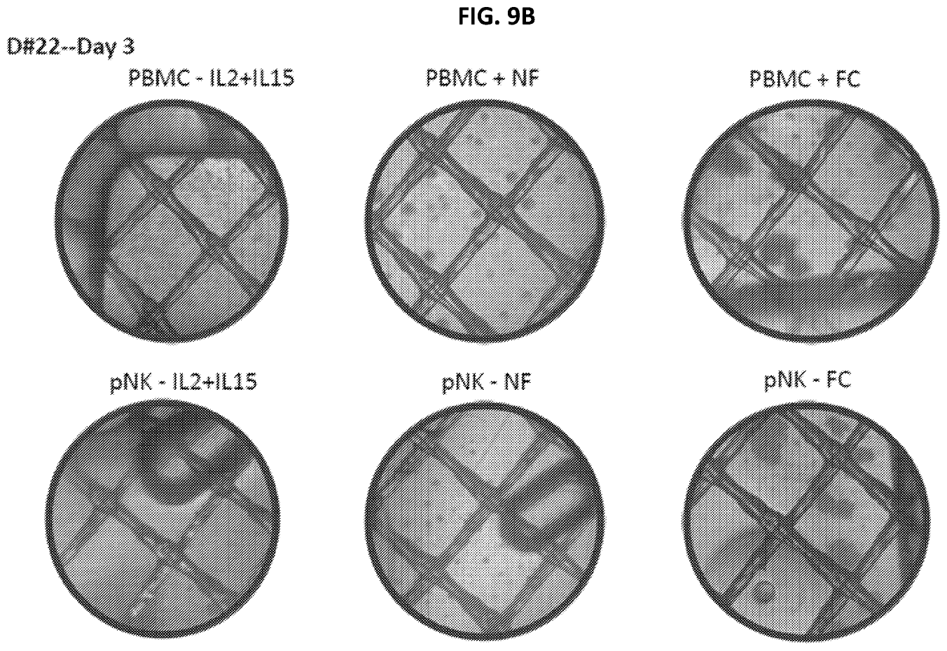

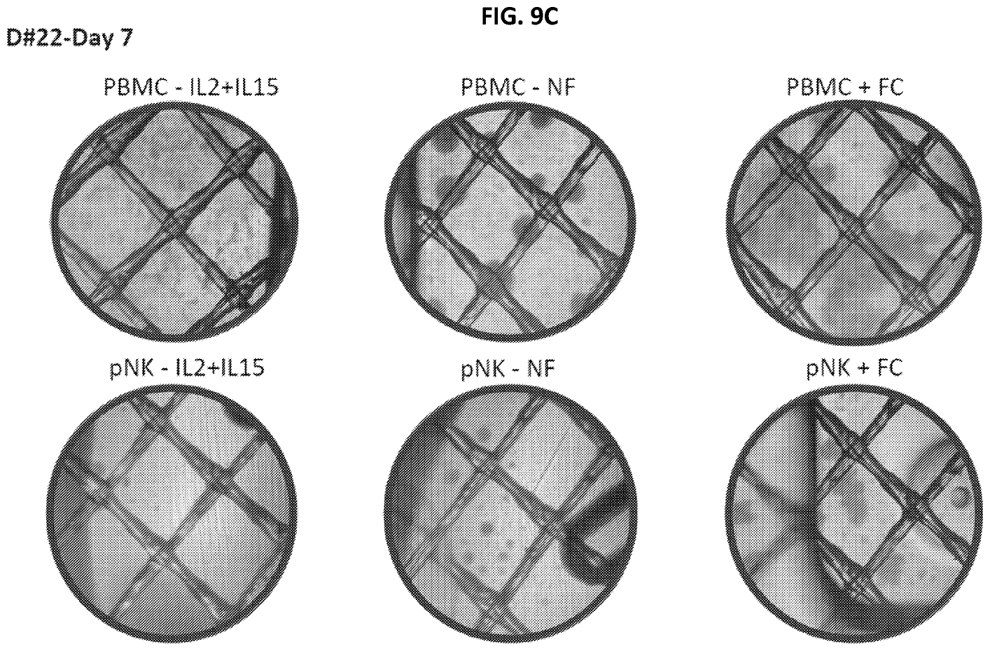

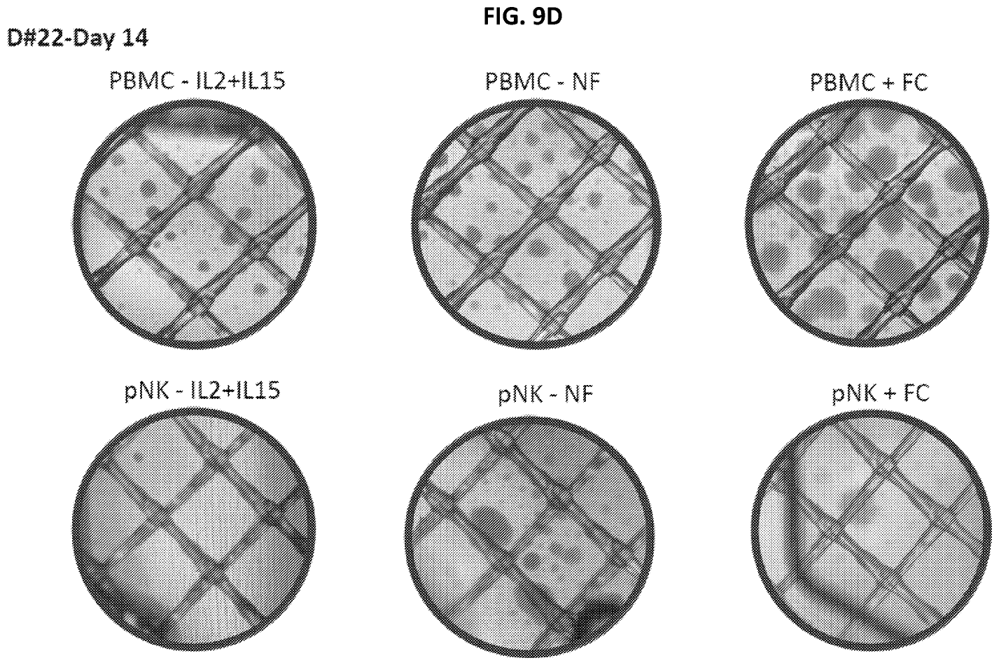

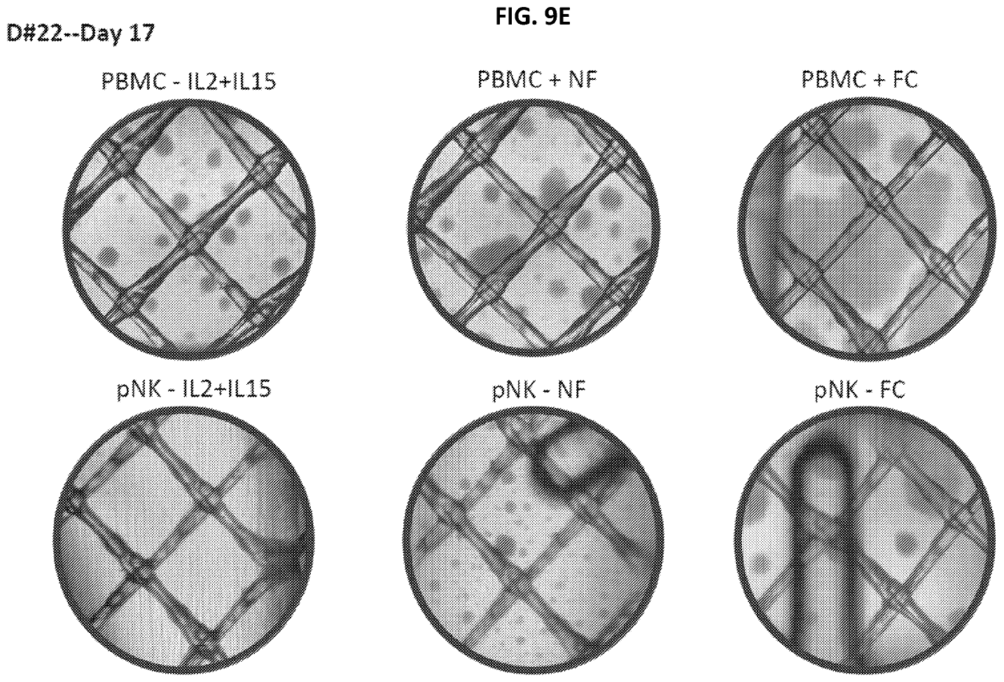

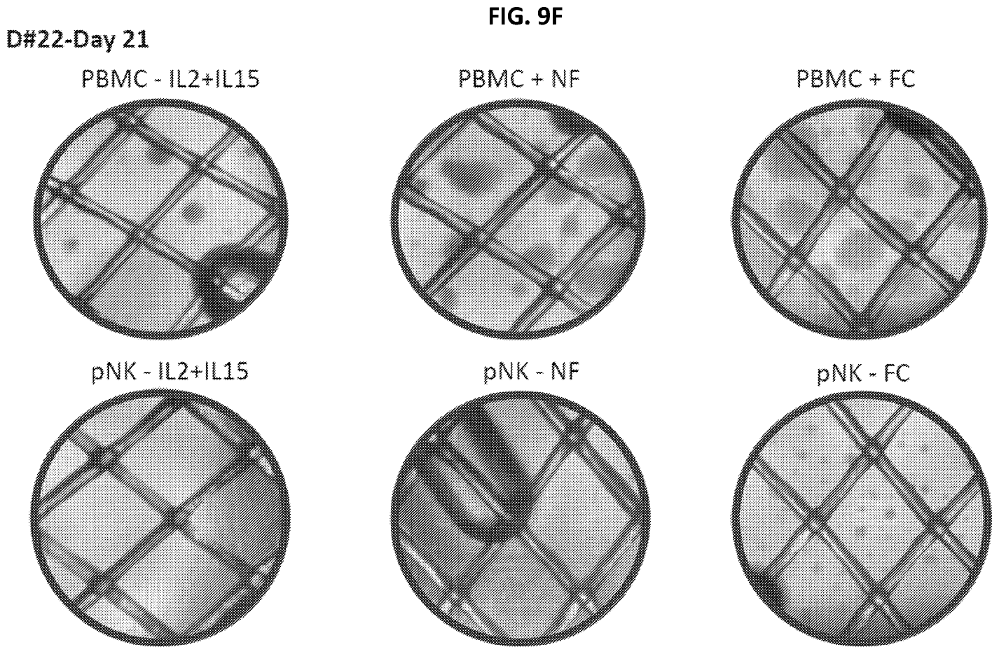

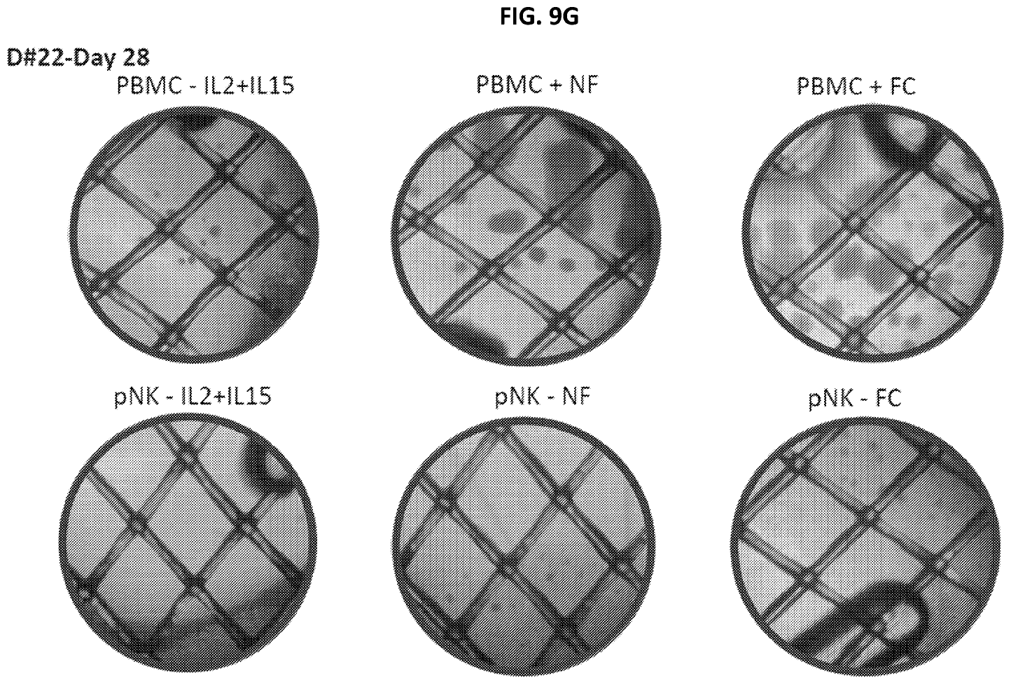

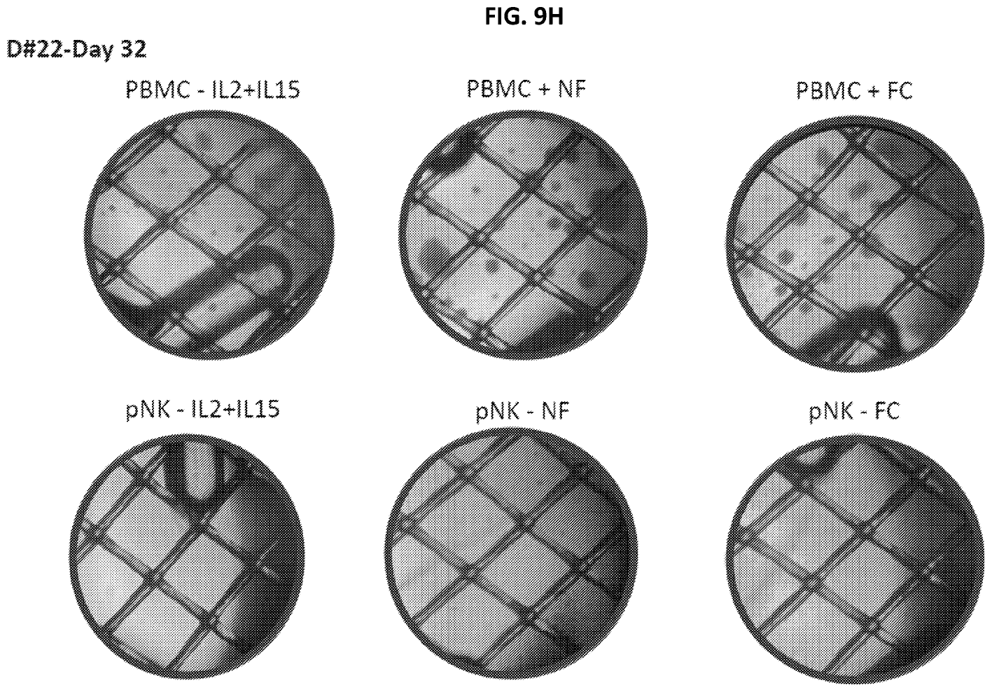

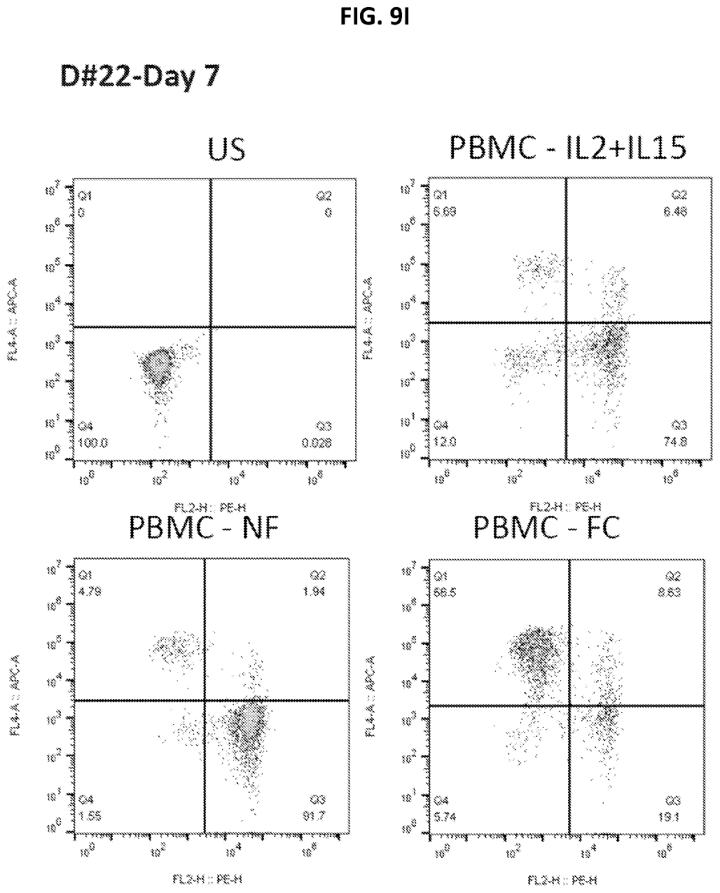

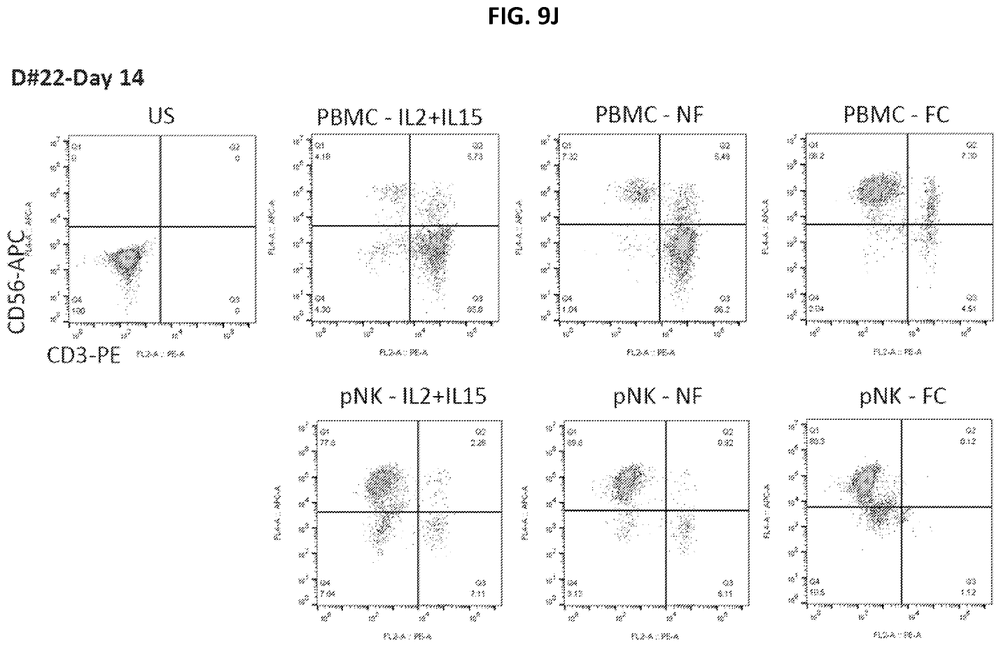

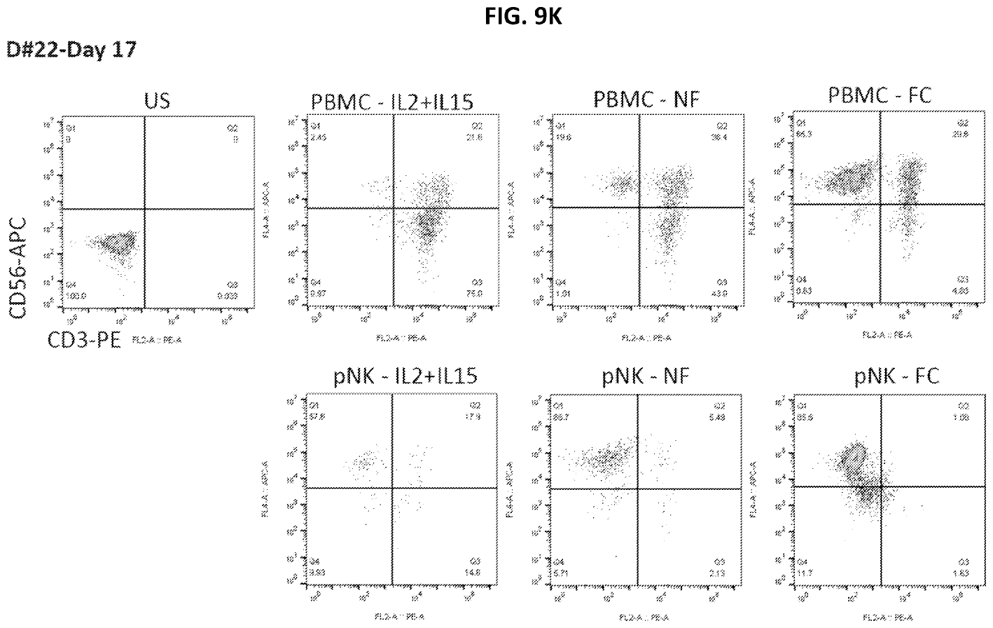

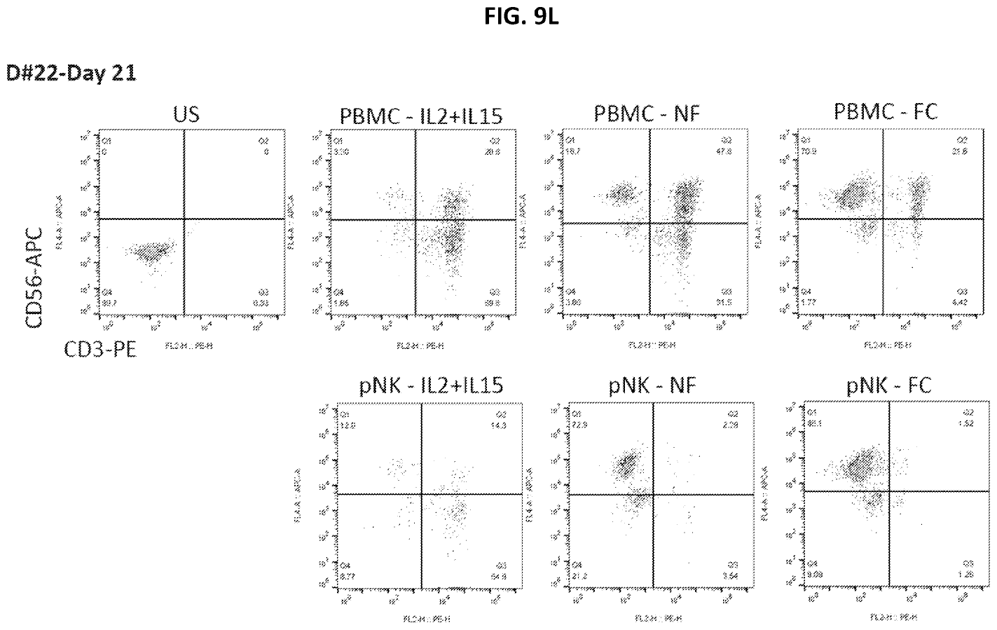

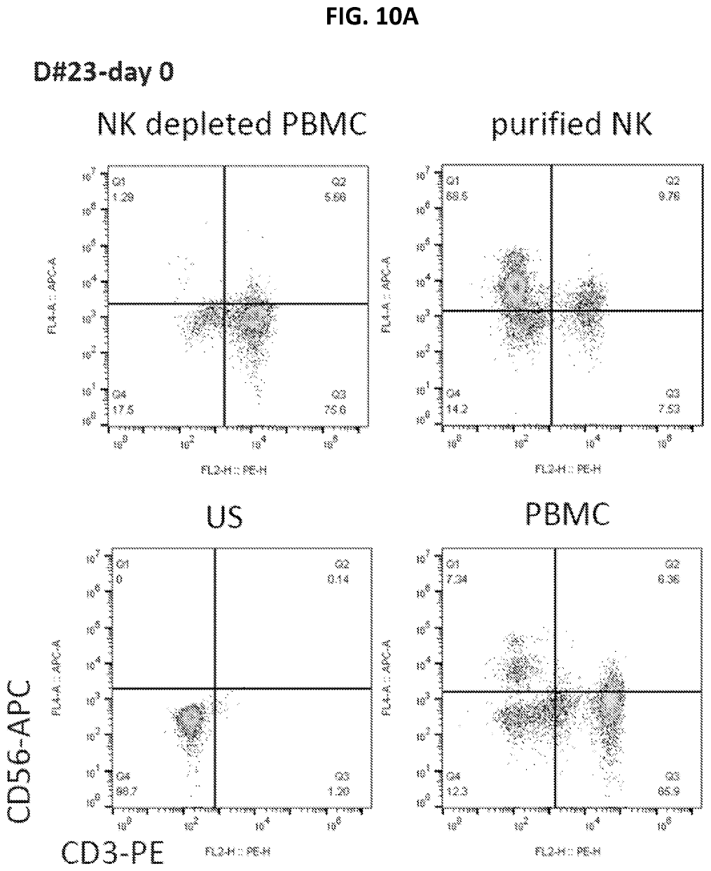

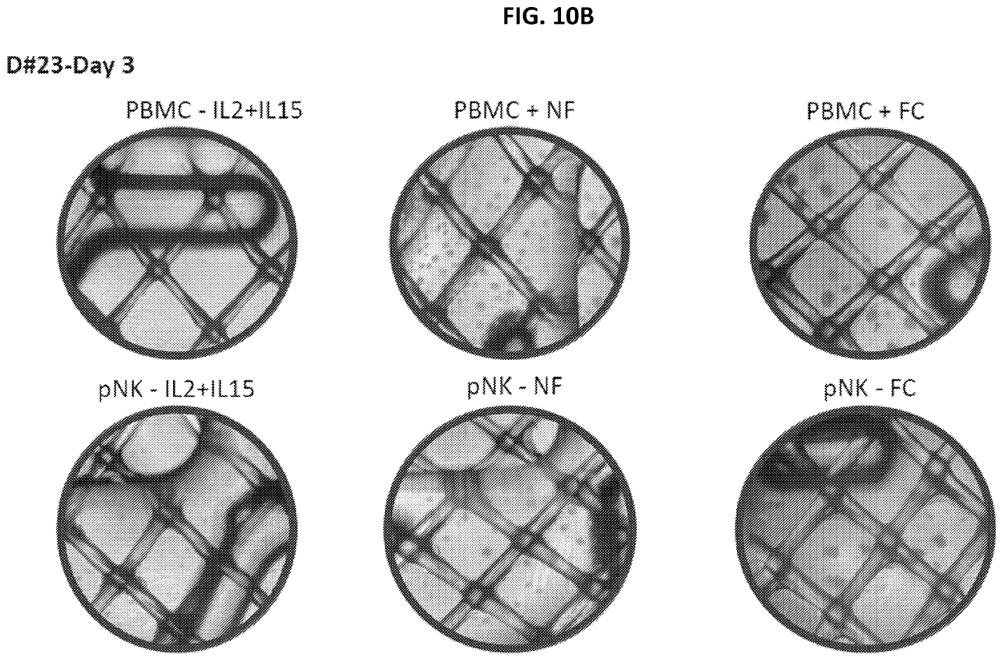

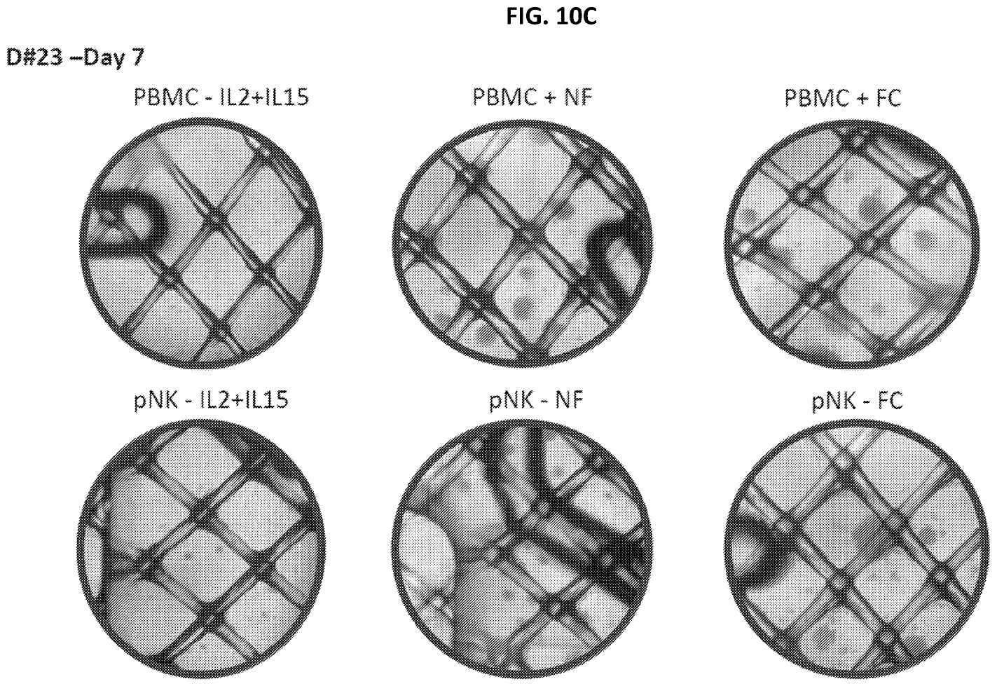

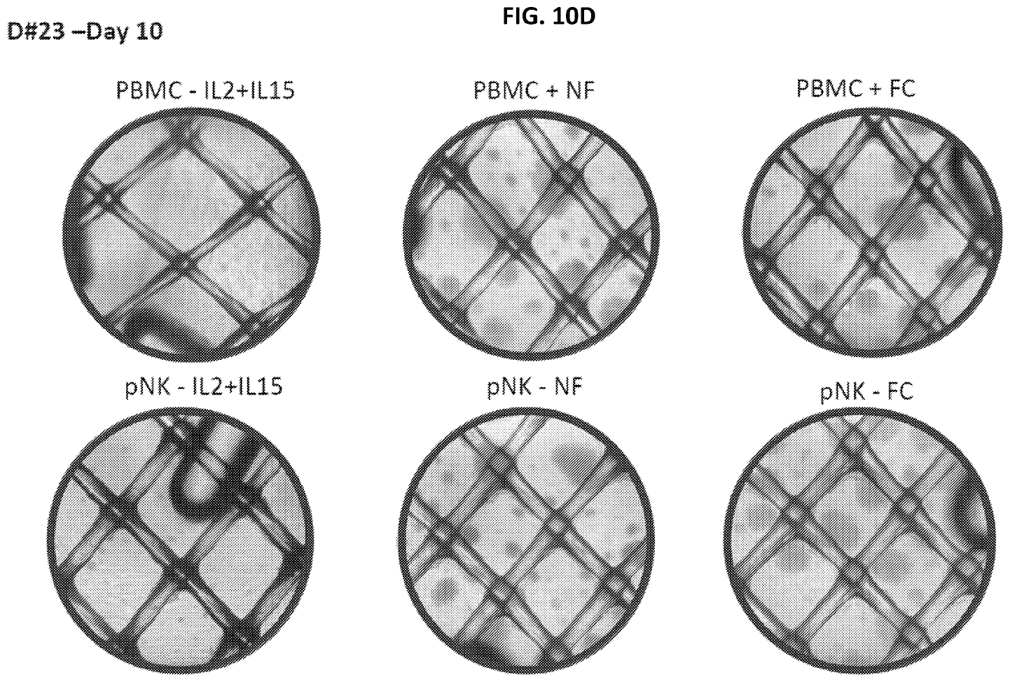

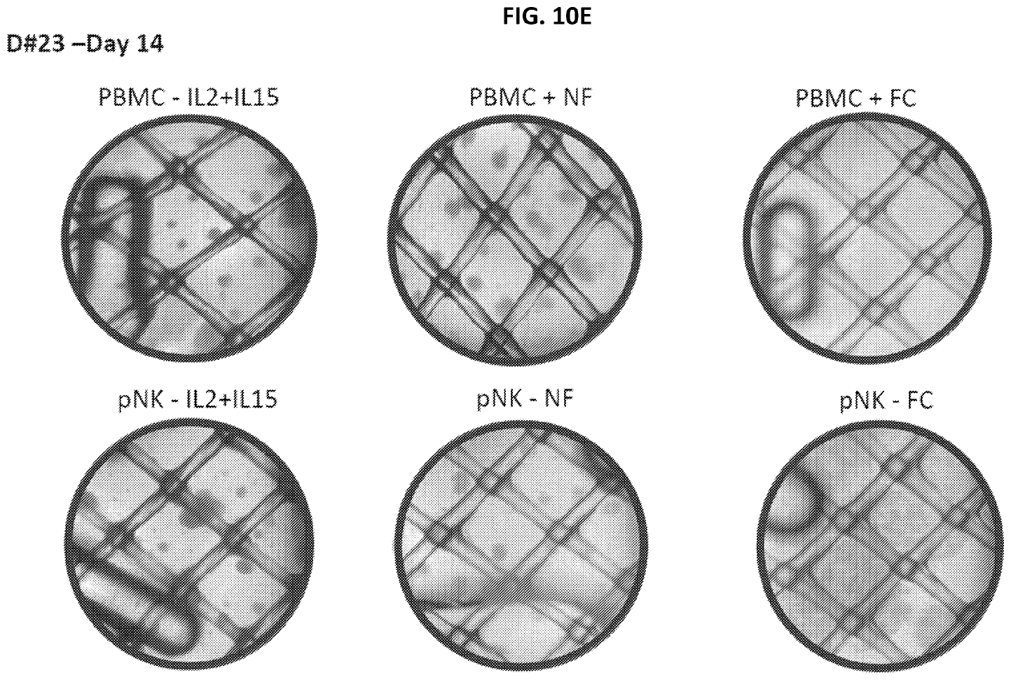

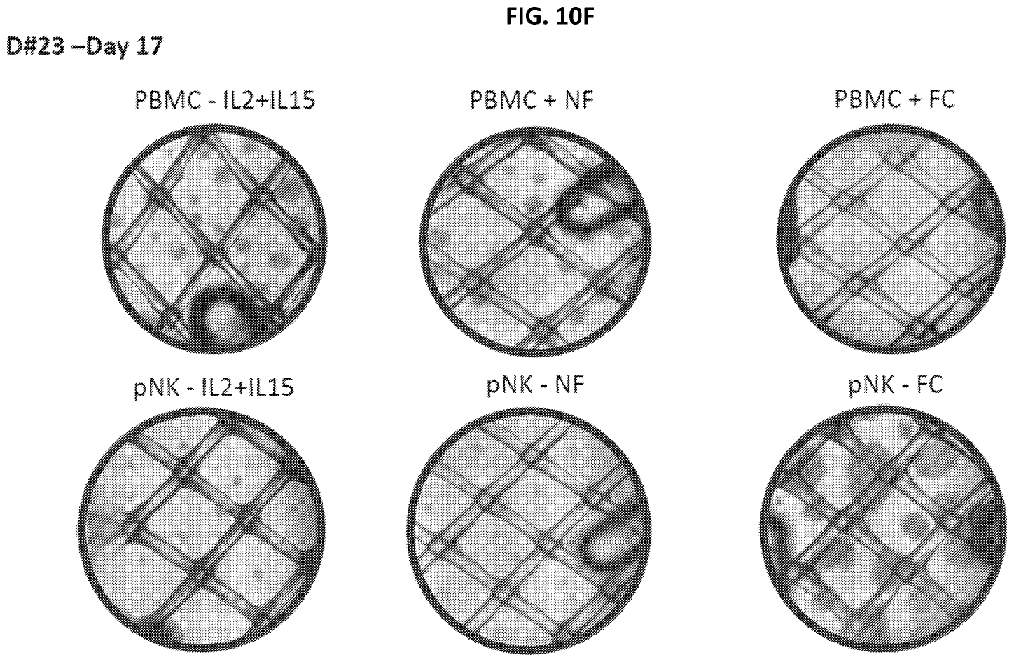

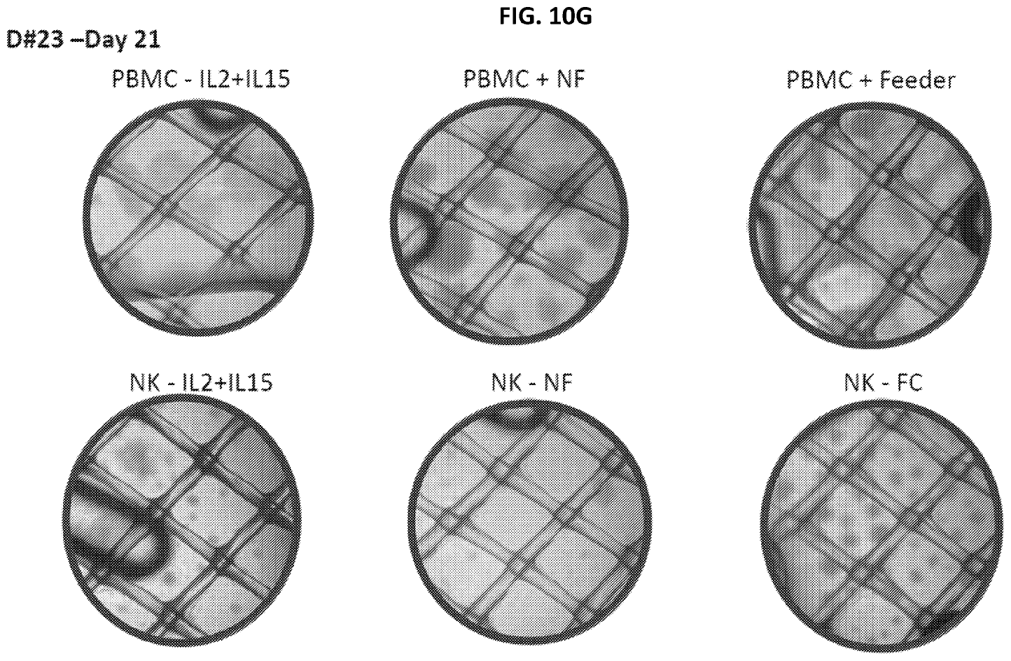

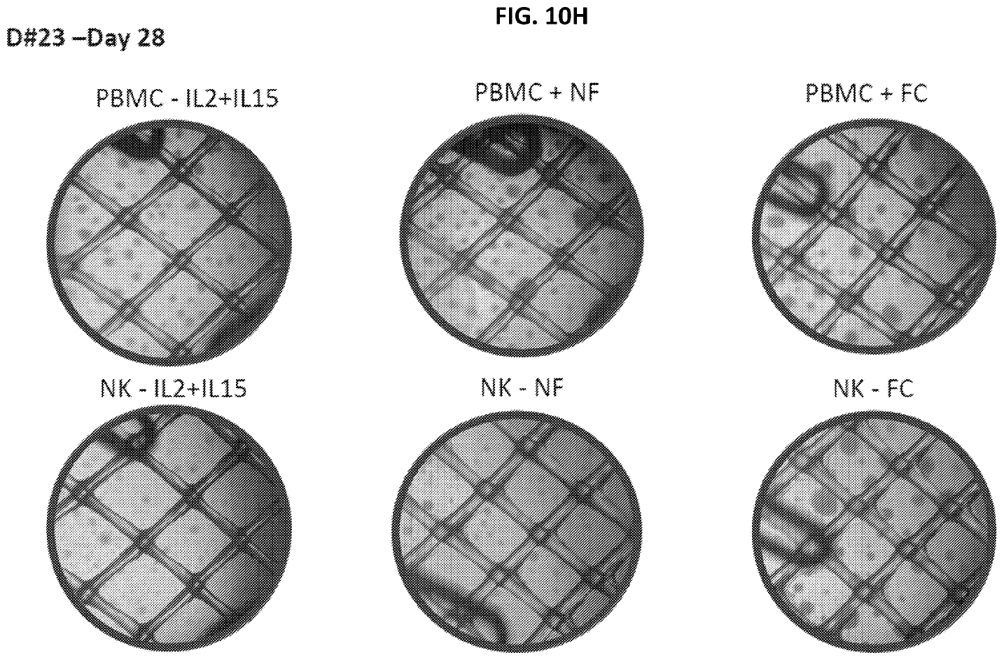

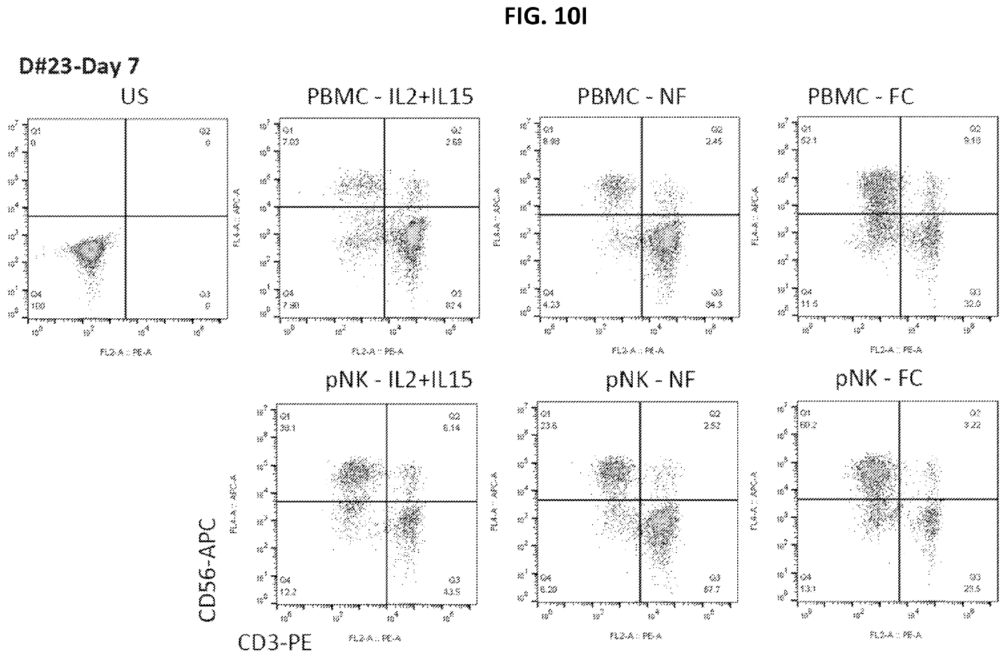

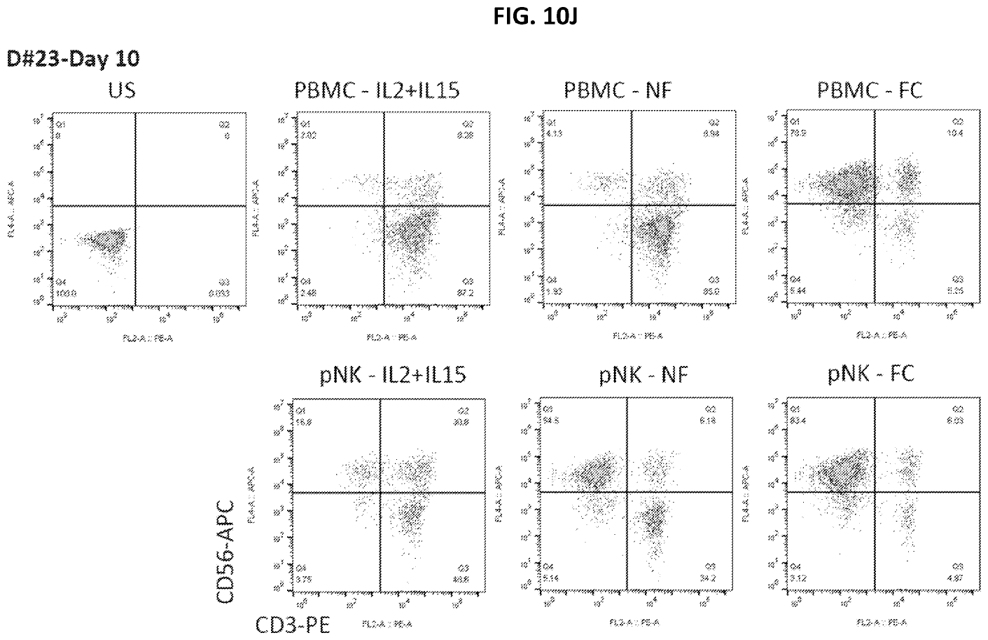

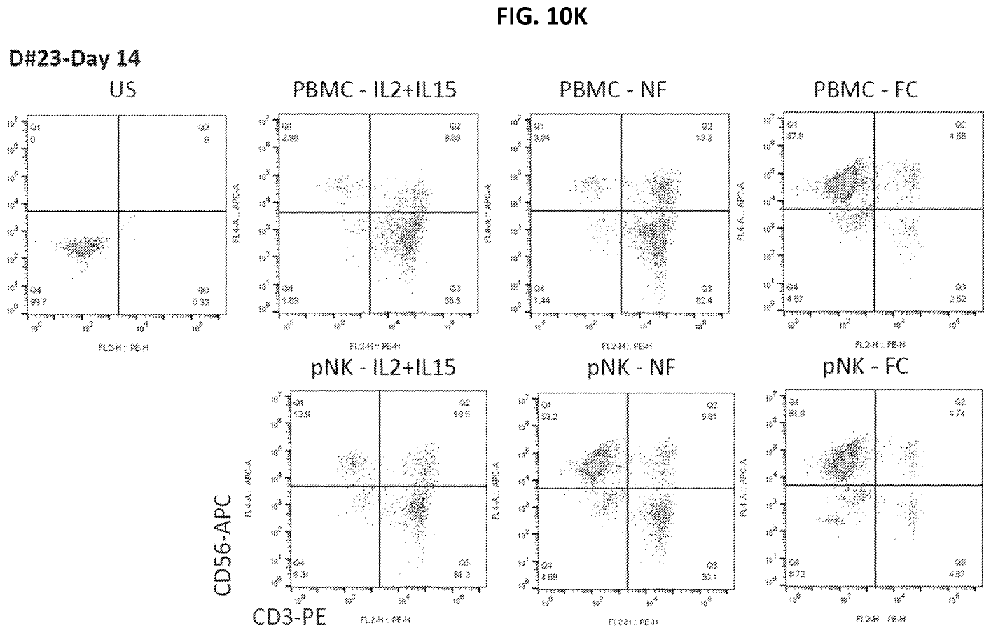

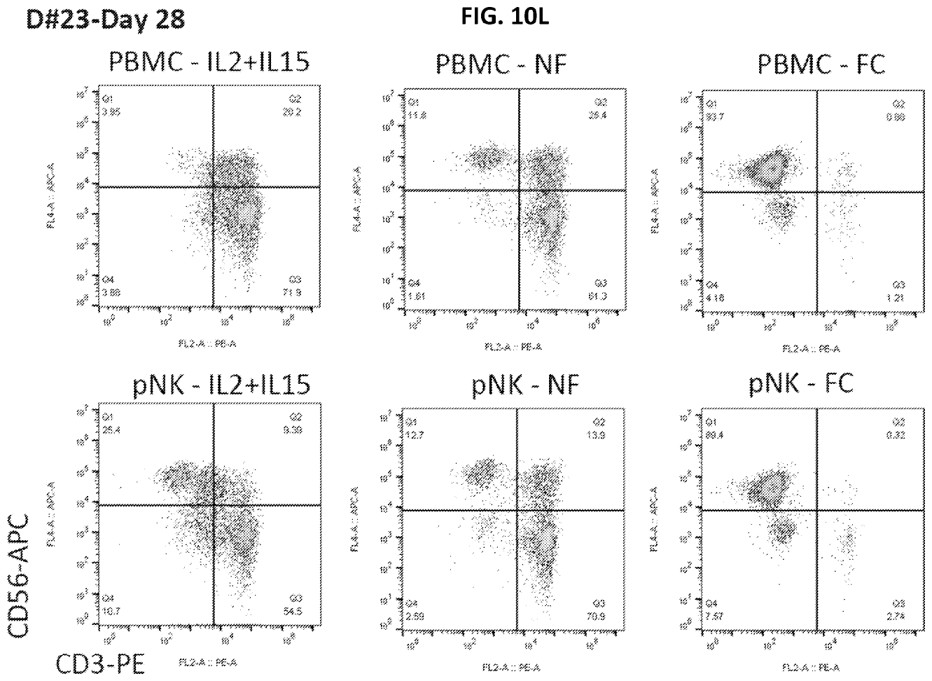

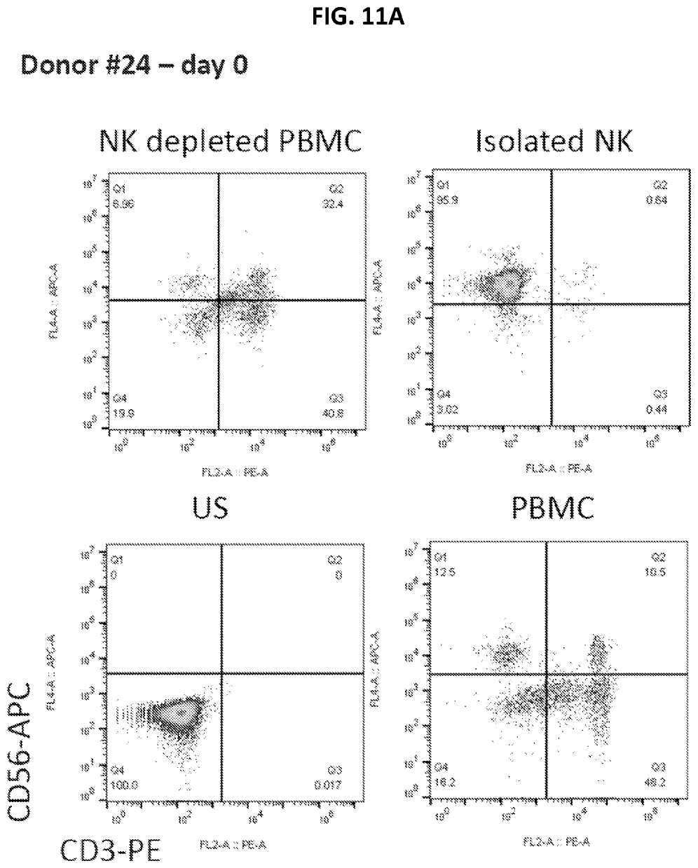

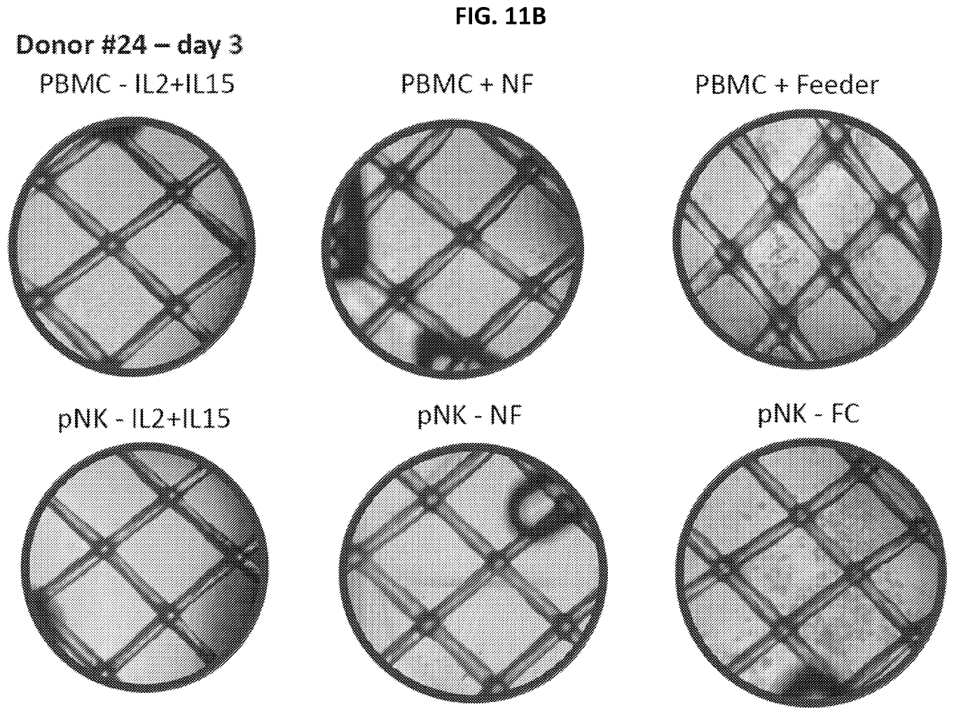

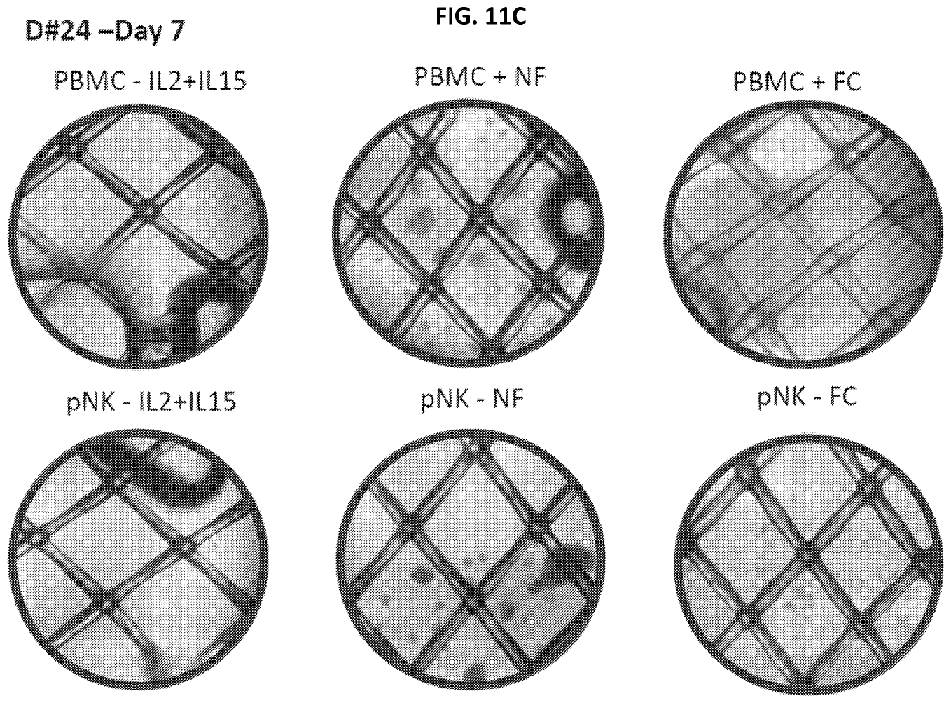

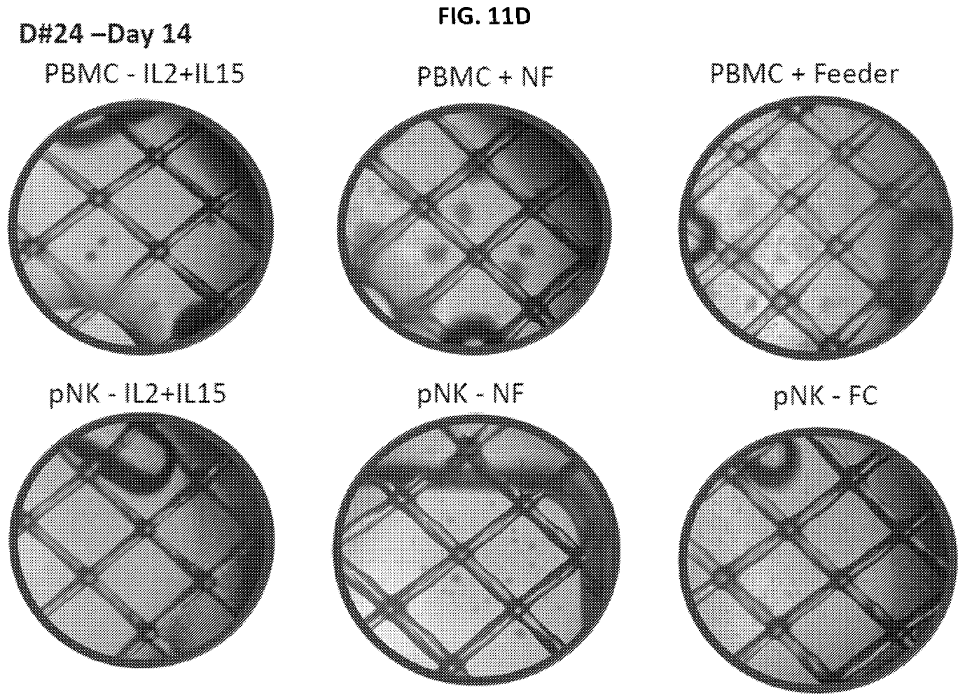

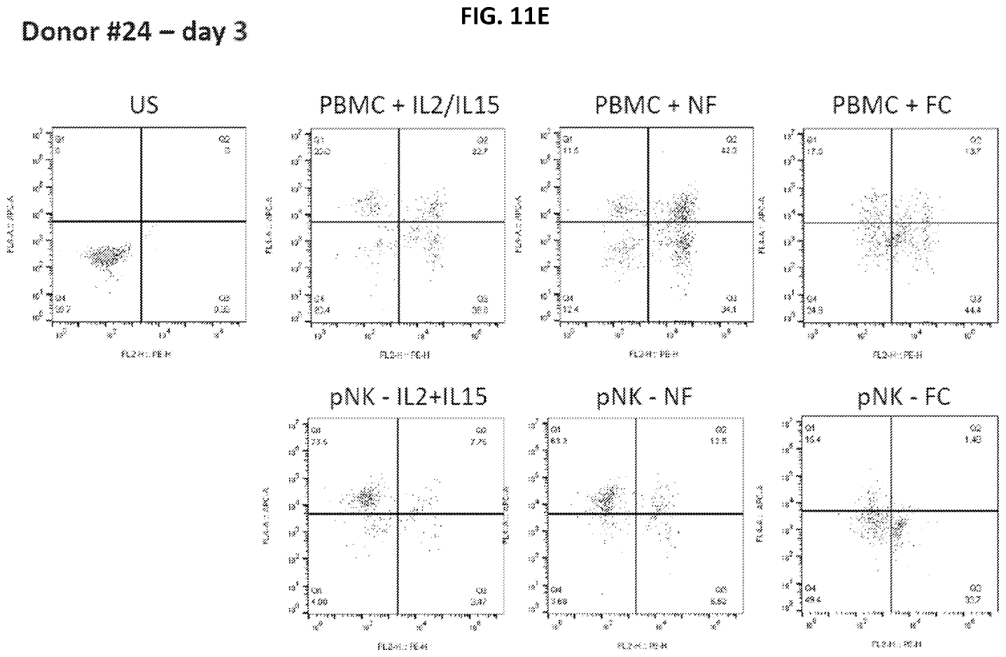

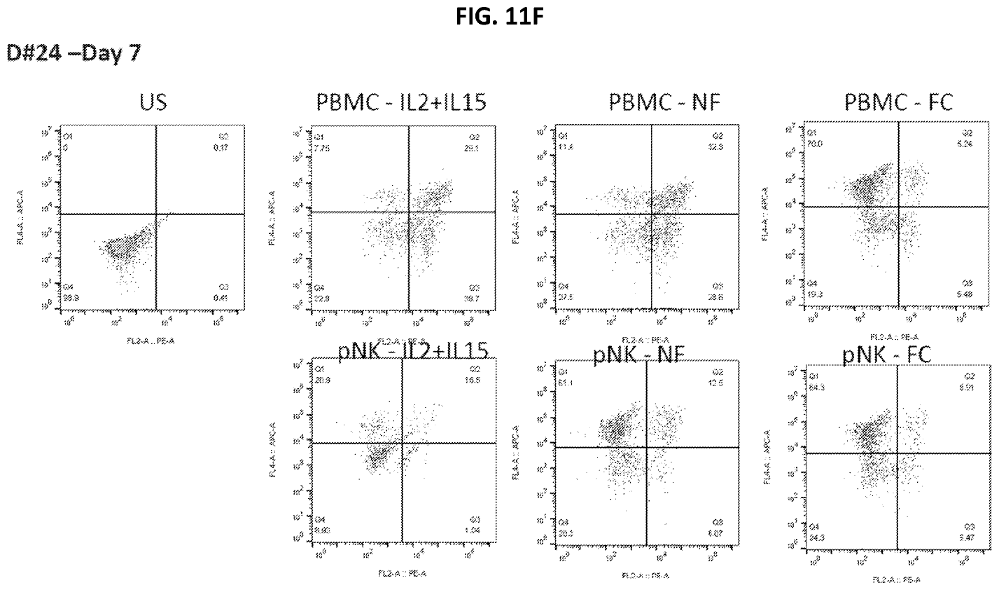

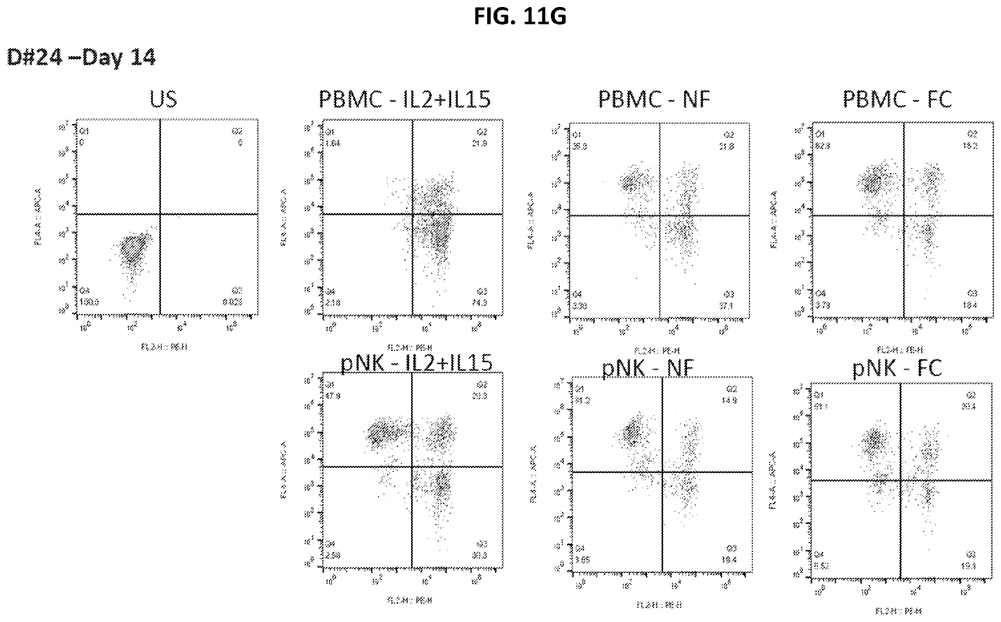

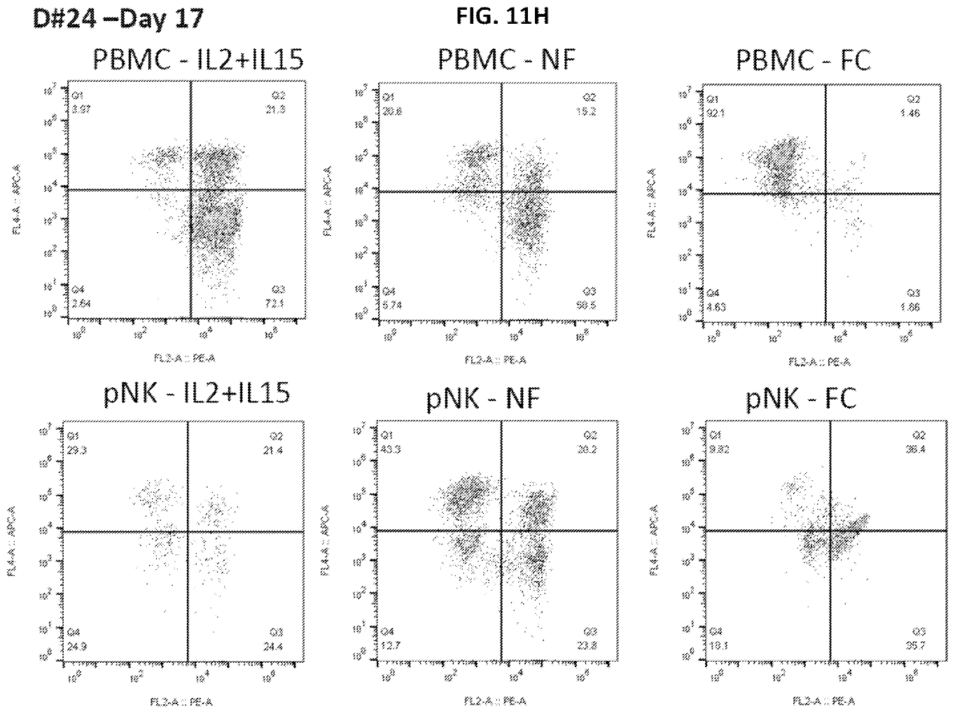

NK cell percentages and proliferation capabilities vary among different individuals. Thus, expansion rate (fold change) and purity of NKs from PBMCs or pNKs was compared between buffy coats of five health donors (FIG. 7). NK cell expansion and immunophenotype characteristics for each individual donor are shown in FIGS. 8-11.

### Example 6

### Characteristics of Expanded NK and CAR-NK Cells Derived from Peripheral Blood

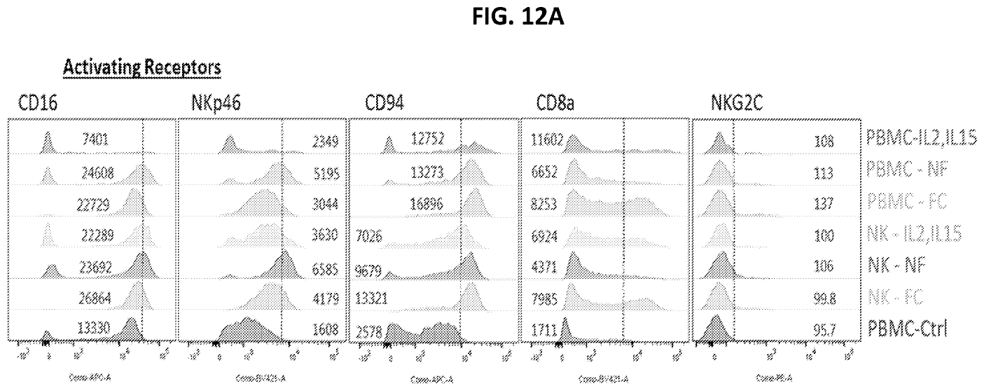

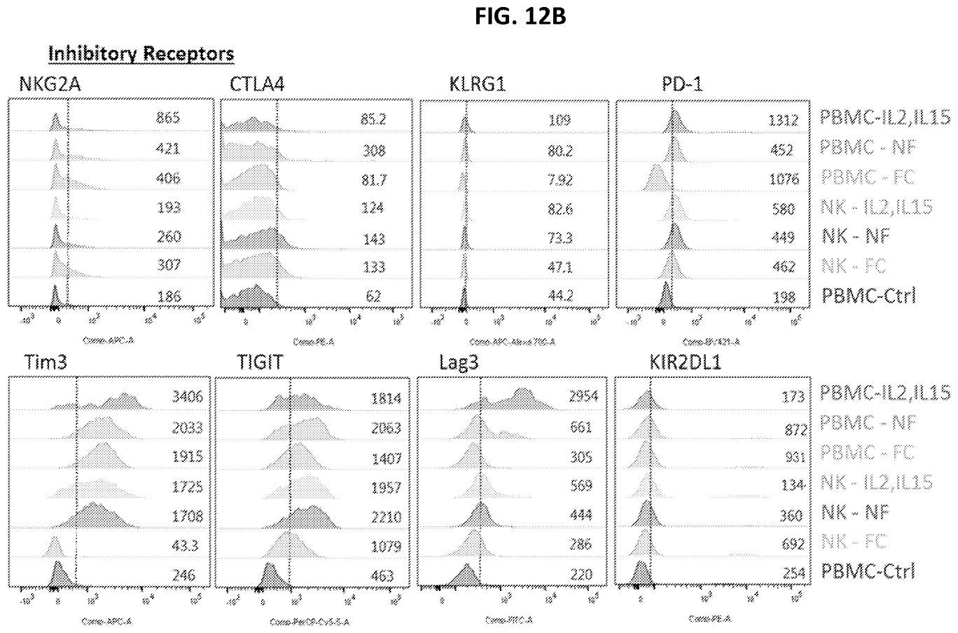

The immunophenotypes of NK cells expanded using cytokines only (IL-2+IL-15), exosomes (the NFC expansion system), or 721.221-mIL-21 cells (the FC expansion system) were assessed and compared. Flow cytometry was used to characterize various activating and inhibitory receptors on the NK cells. The activating receptors included CD16, NKp46, CD94, CD8a, and NKG2C (FIG. 12A). The inhibitory receptors included NKG2A, CTLA-4, KLRG1, PD-1, TIM-3, TIGIT, LAG-3, and KIR2DL1 (FIG. 12B). Expression of the activating and inhibitory receptors on expanded NK cells was similar between expansion methods. However, expression of inhibitory receptors (e.g., TIM-3 and LAG-3) on NK cells expanded using the NFC expansion system were lower as compared with cytokines alone. Conversely, expression of activating receptors (e.g., NKP46) was higher as compared with both the FC expansion system and cytokines alone. In summary, NFC system-expanded PBNK cells had similar expression of surface receptors as compared with FC system-expanded PBNK cells.

### Example 7

### Functional Characteristics of Expanded NK and CAR-NK Cells Derived from Peripheral Blood

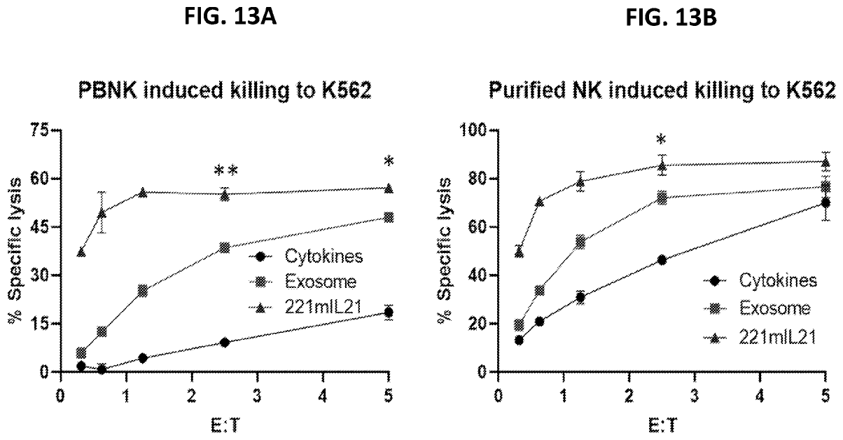

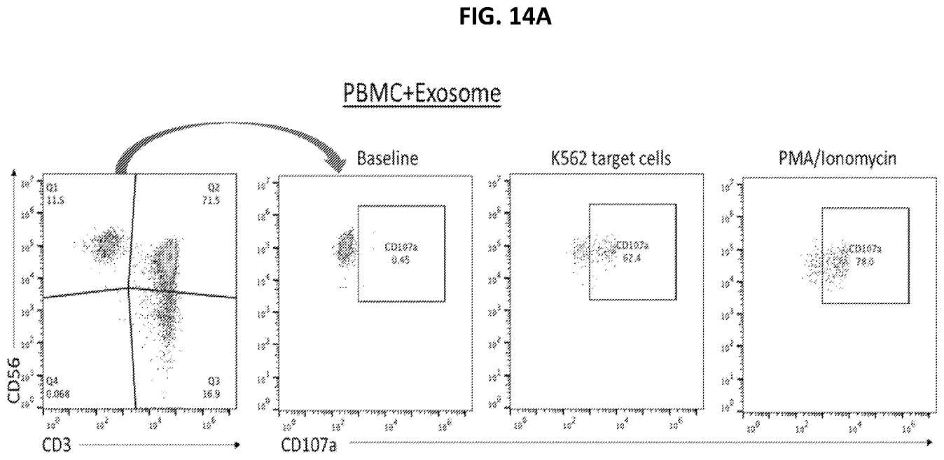

The 4-hour chromium-51 (⁵¹Cr) release assay was used to test whether NK cells expanded using the NFC expansion system could kill target cells in vitro. The results showed that NK cells effectively kill NK-sensitive K562 cells (FIG. 13). NK cells expanded using the NFC expansion system showed increased cytotoxicities as compared to NK cells expanded using IL-2 and IL-15.

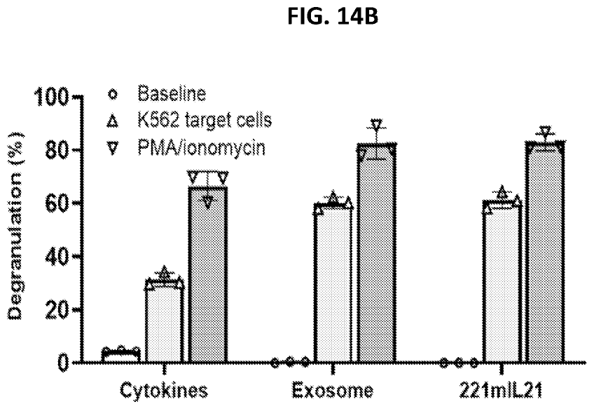

To confirm this observation, the CD107a assay was used to determine cell surface levels of CD107a after NK degranulation. A similar percentage of CD107a degranulation was observed in exosome-expanded NK cells as compared to 721.221-mIL-21-expanded NK cells (FIG. 14), indicating possible unpolarized, spontaneous degranulation on exosome-expanded NK cells. Thus, NK cells expanded using the NFC system or the FC expansion system showed similar degranulation capability and killing activities.

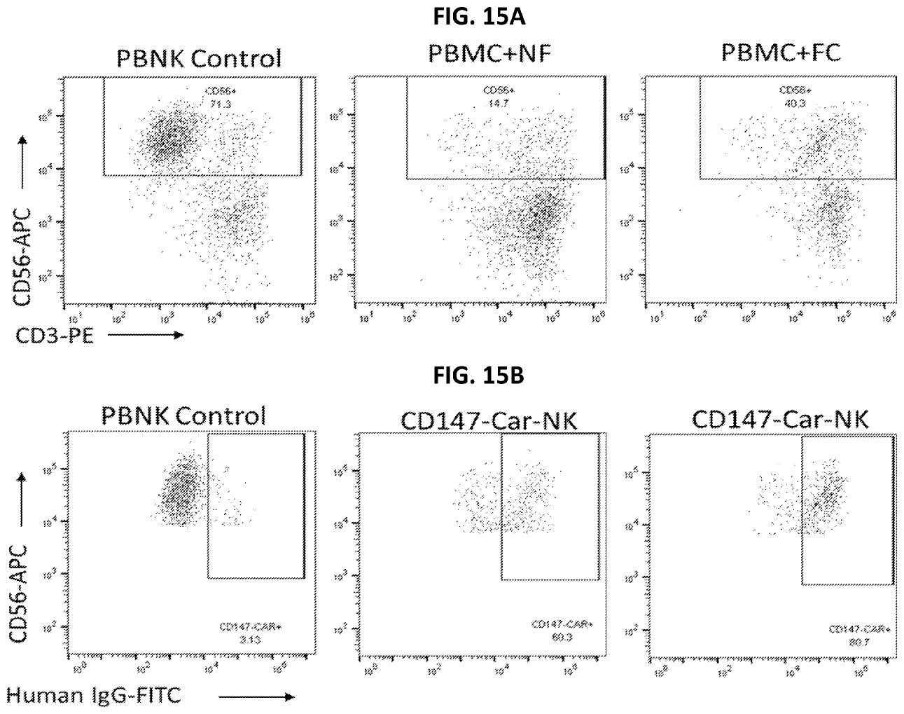

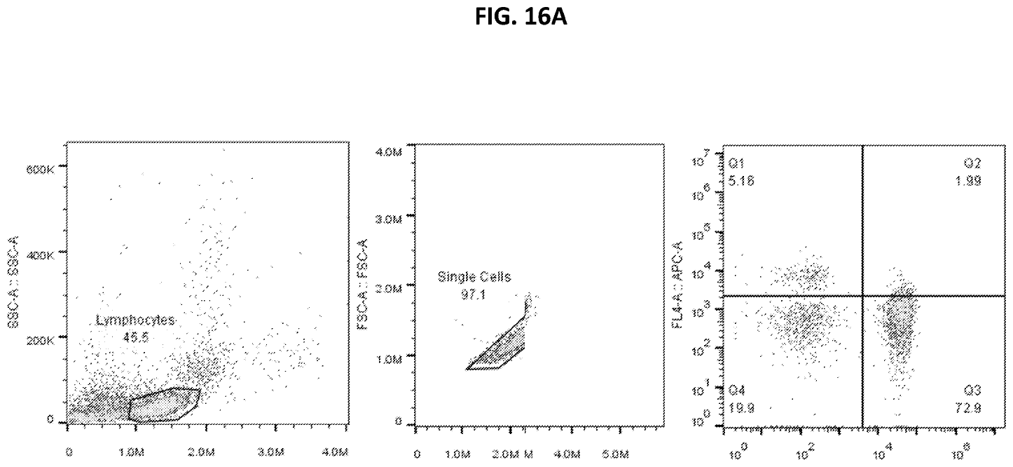

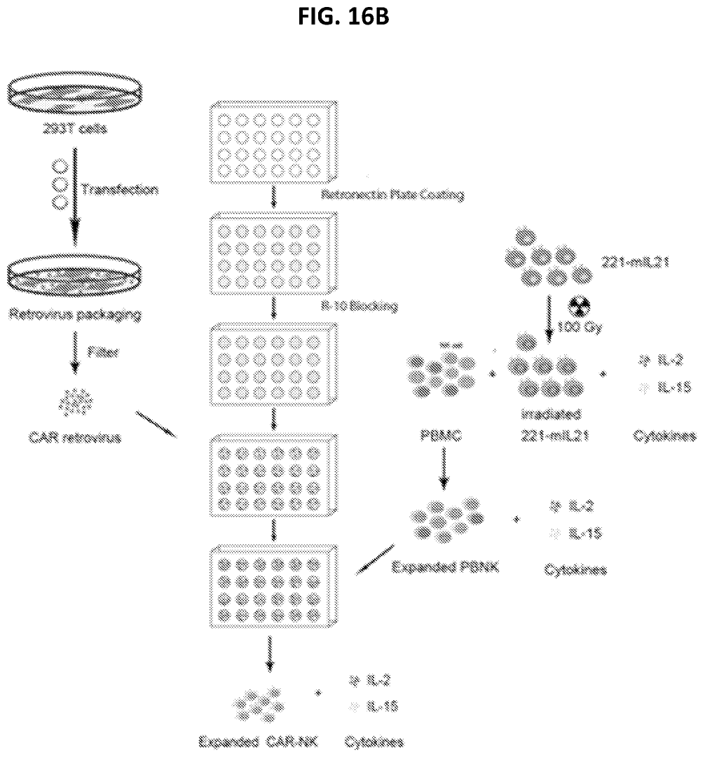

CAR-NK immunotherapy is a promising strategy for tumor treatments and has been evaluated in clinical trials. Whether the NFC expansion system could expand CAR-modified NK cells at rates and purities similar to the FC expansion system was assessed. To expand CAR-NK cells ex vivo, unfractionated PBMCs were stimulated for 7 days with either the NFC system or 721.221-mIL-21 feeder cells in the presence of soluble IL-2 and IL-15. At day 7, expanded NK cells were transduced with CD147-CAR retrovirus. CD147-CAR transduction was measured using flow cytometry. The percentage of CD147-CAR-positive NK cells stimulated using the NF cell system (60.3%) was slightly lower than that of CD147-CAR-NK cells stimulated using 721.221-mIL-21 feeder cells (80.7%). The results demonstrated that the NFC system had similar CAR-NK cell expansion capability compared to that of 721.221-mIL-21 feeder cells (FIG. 15).

### Example 8

### Validation of Non-Feeder Cell System

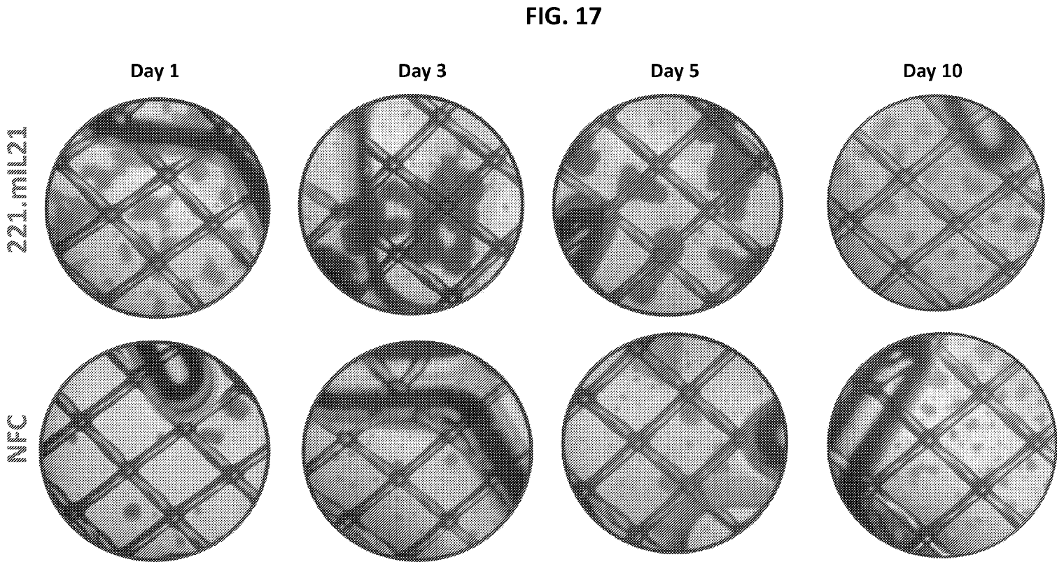

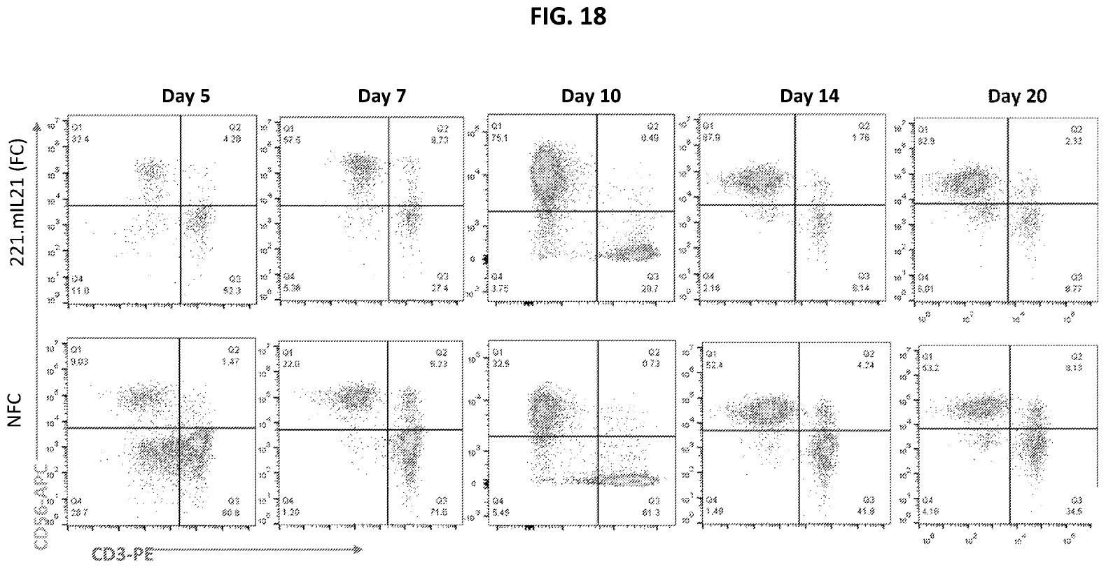

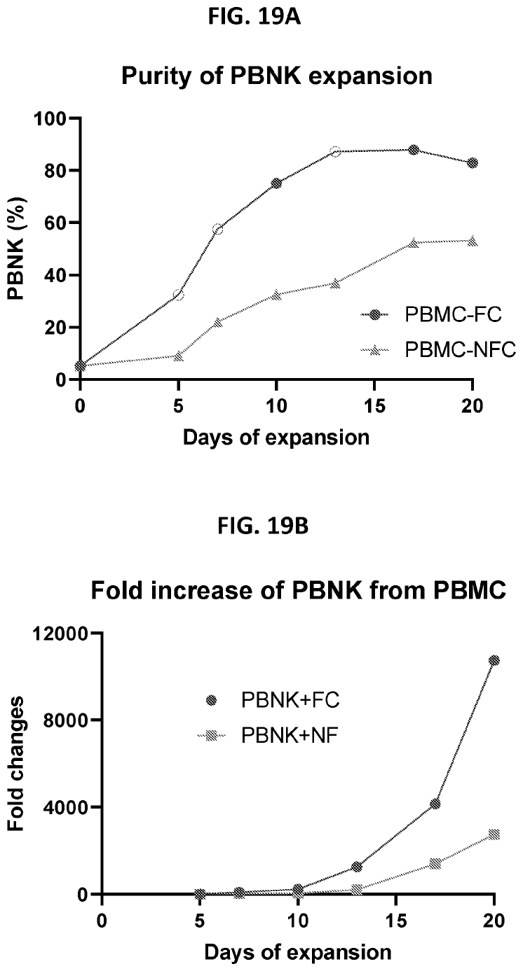

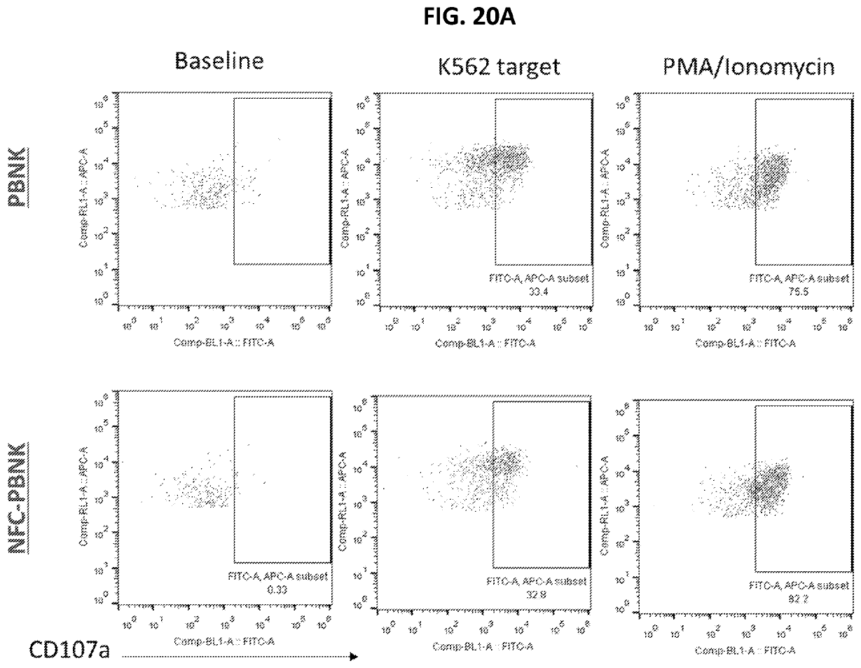

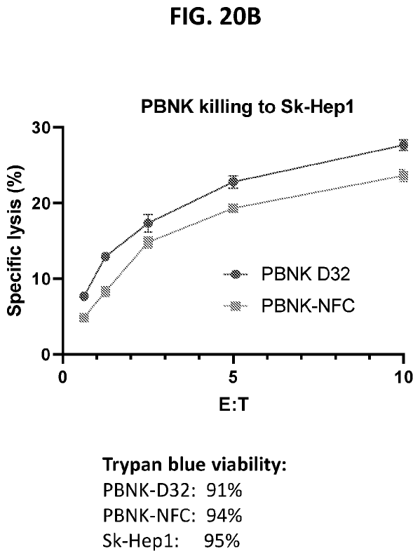

Additional experiments were performed for comparison of the 721.221-mIL-21 feeder cell (FC) system and non-feeder cell (NFC (exosome)) system, using the methods described in Example 1. NK cell expansion from PBMCs from a donor (donor 32) using FC and NFC systems were performed over 20 days (FIGS. 17-19). Function of the cells expanded with each system were assessed by CD107a degranulation assay (FIG. 20A) and ⁵¹Cr killing assay (FIG. 20B). PBNK expanded using each system showed similar activity in these assays

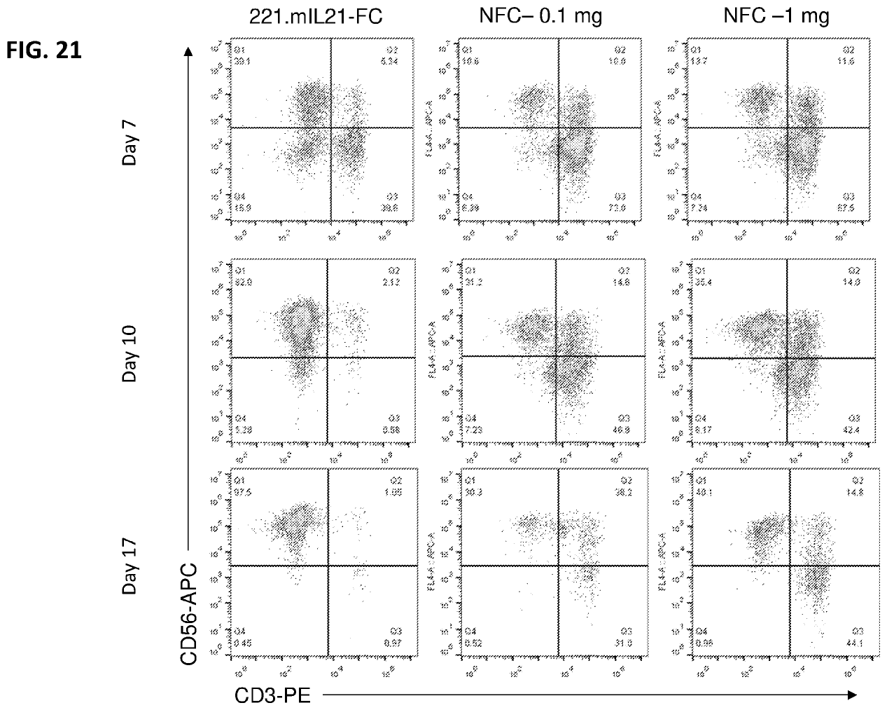

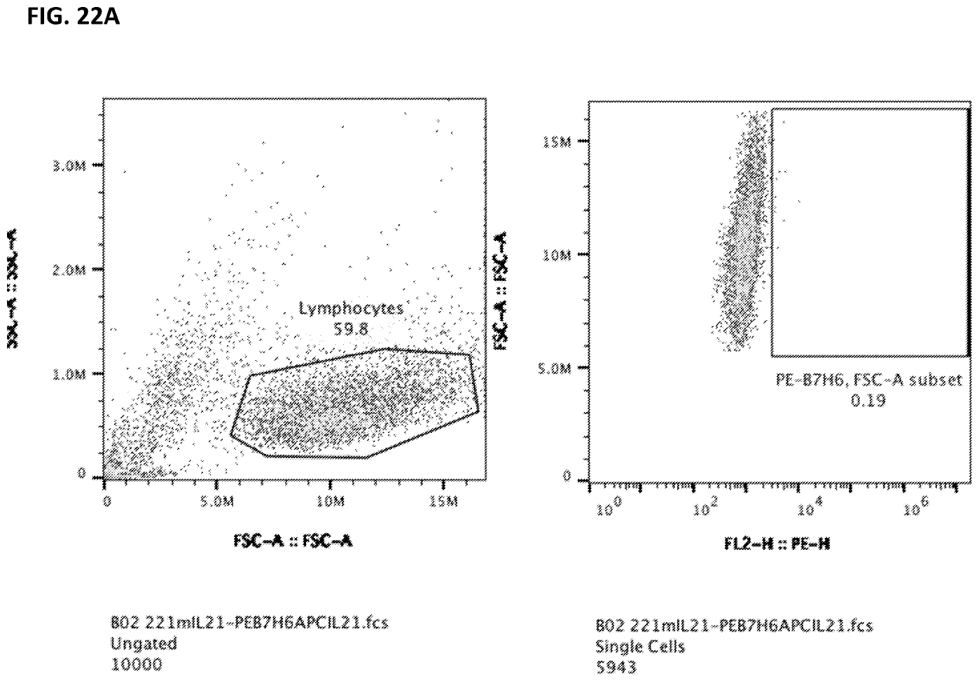

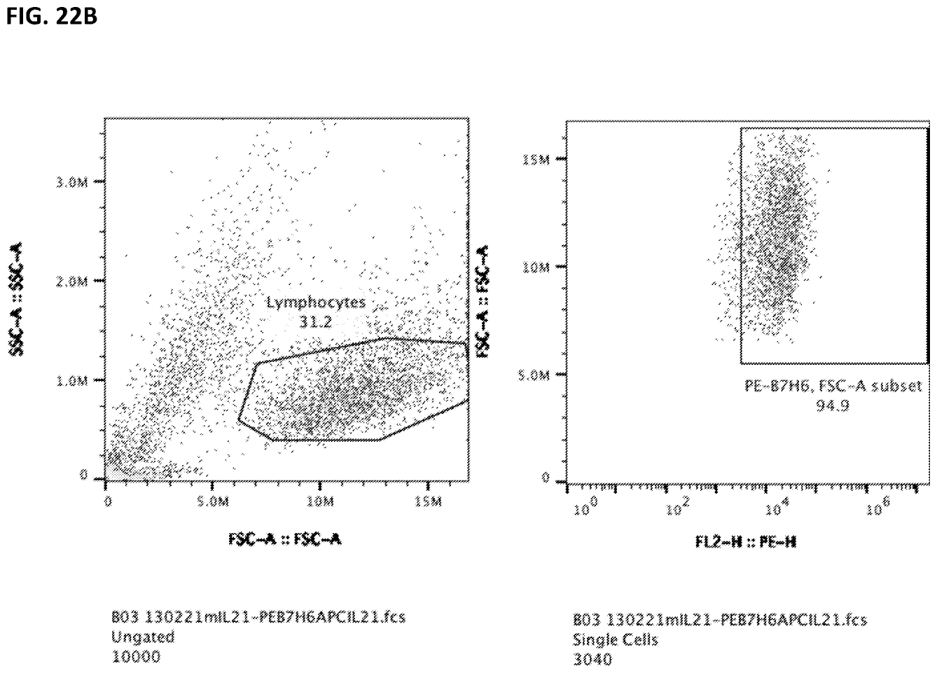

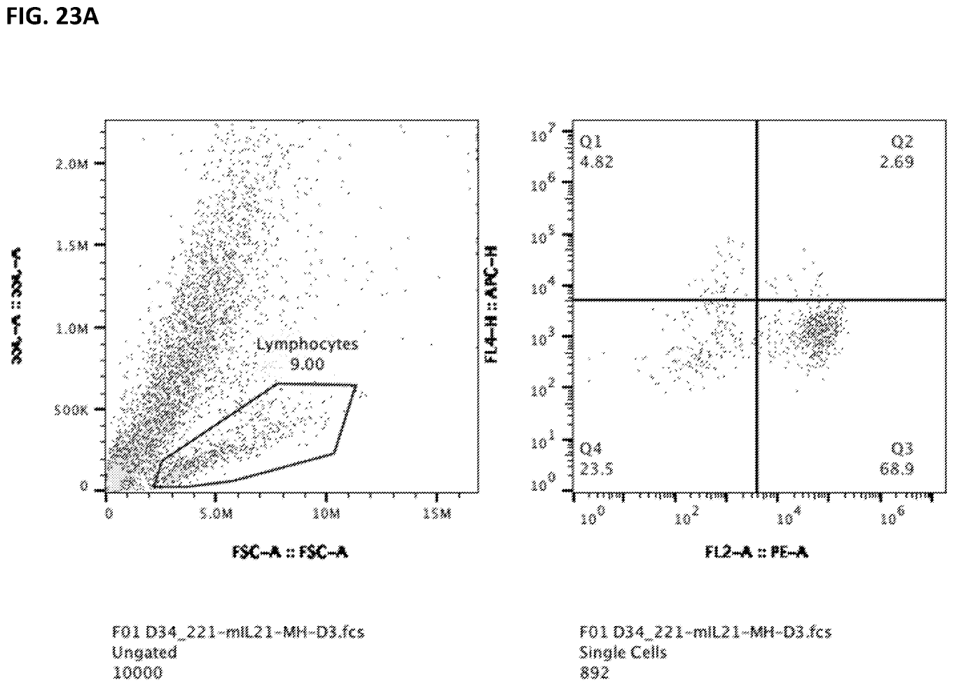

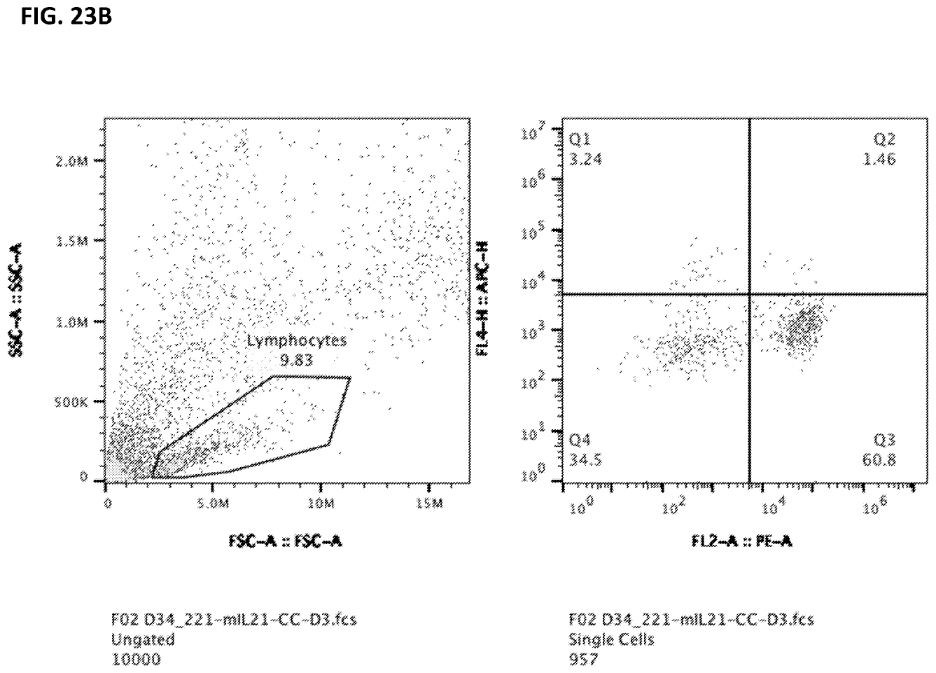

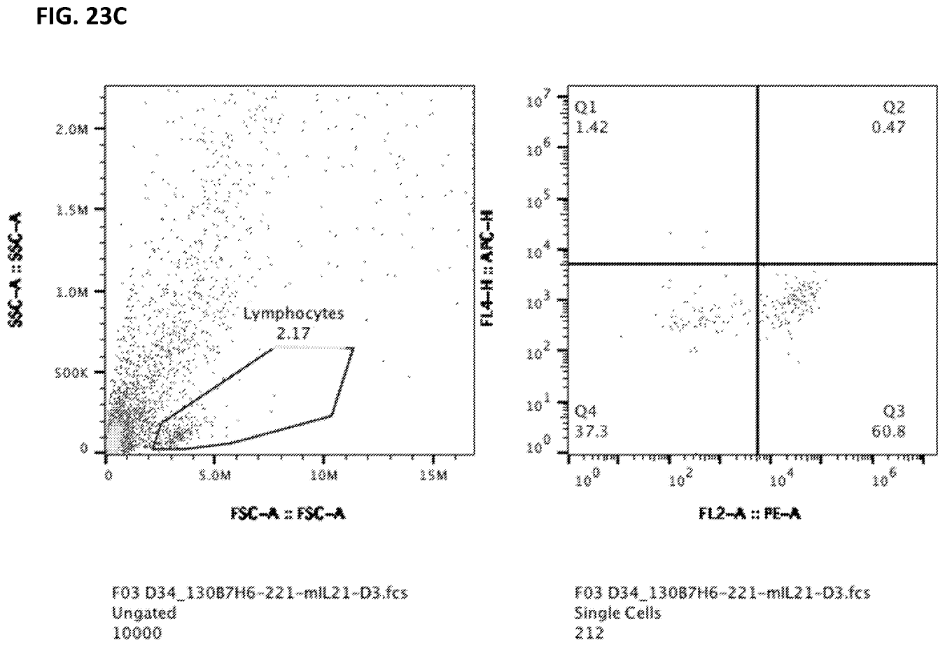

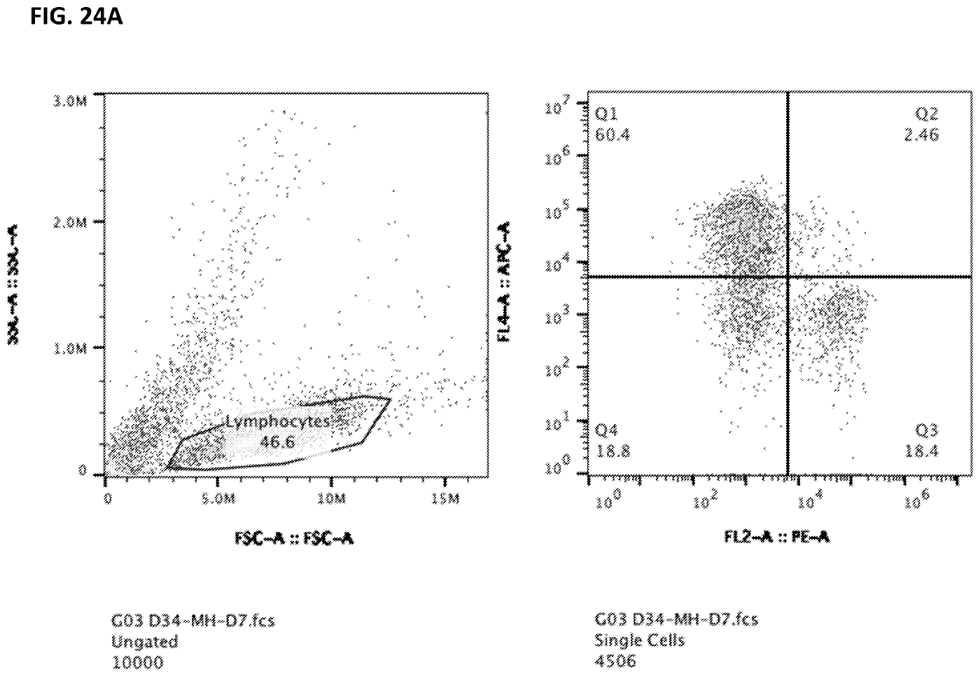

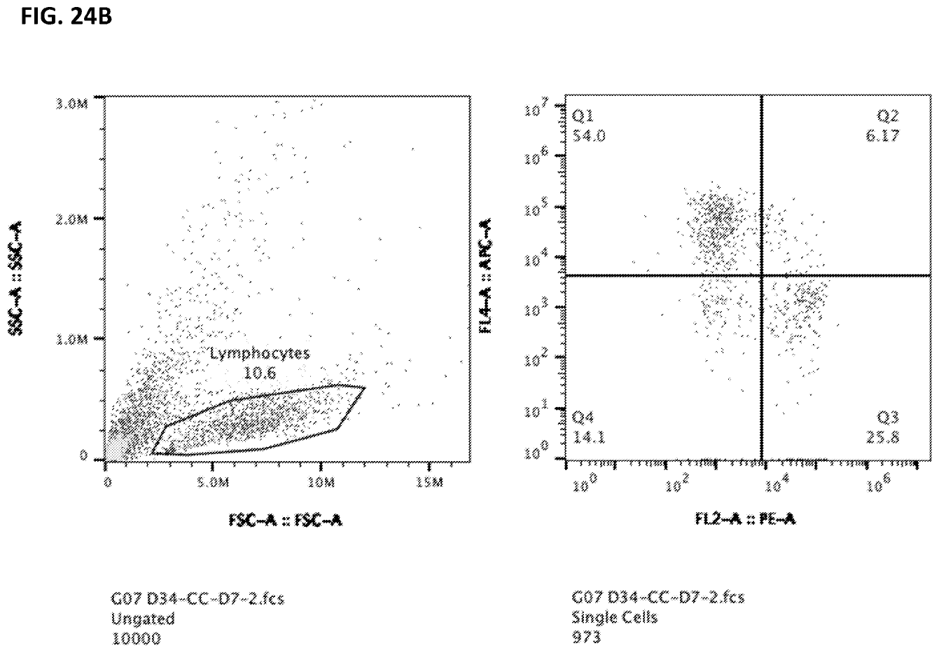

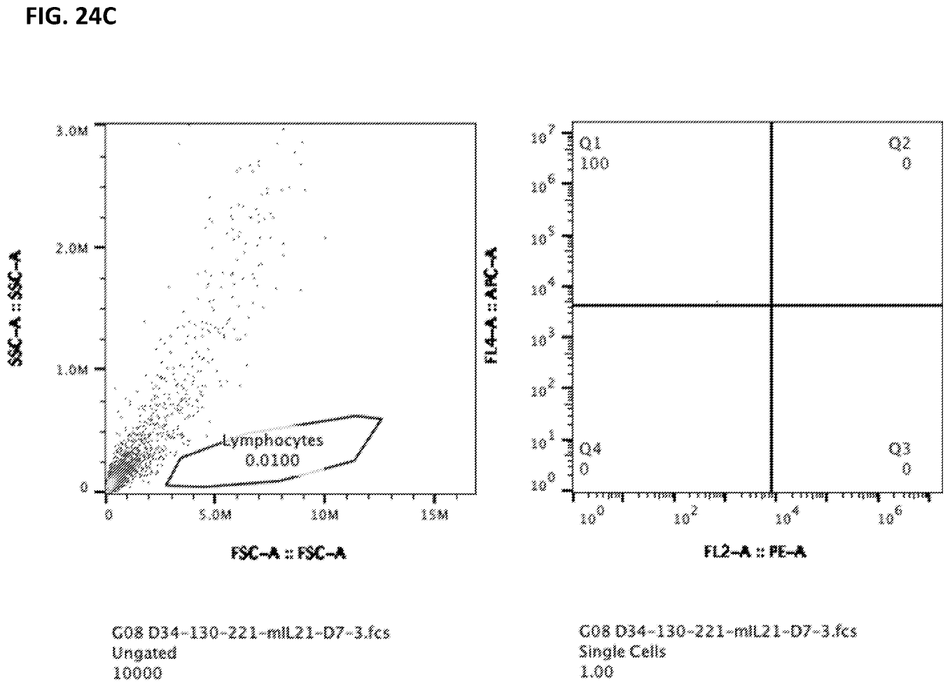

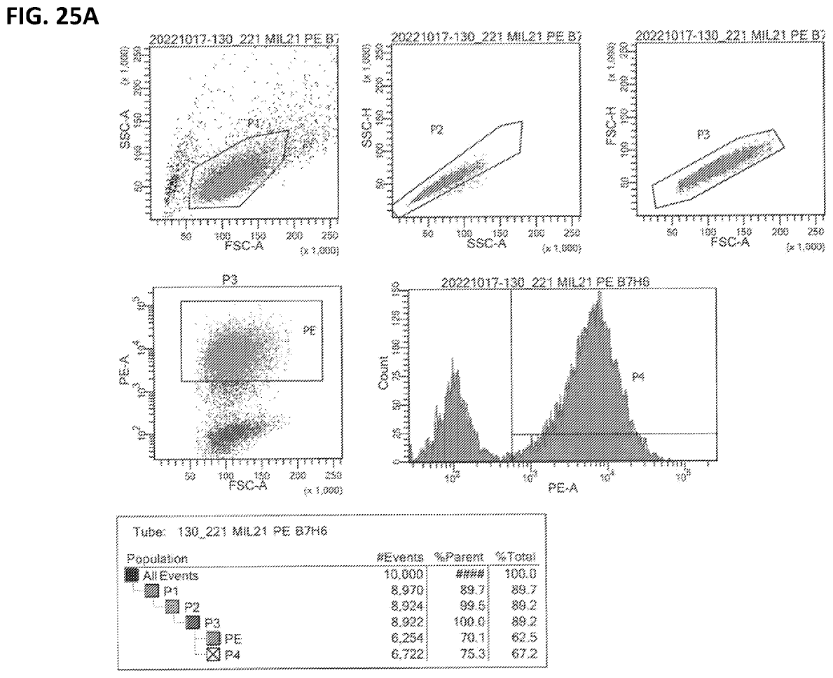

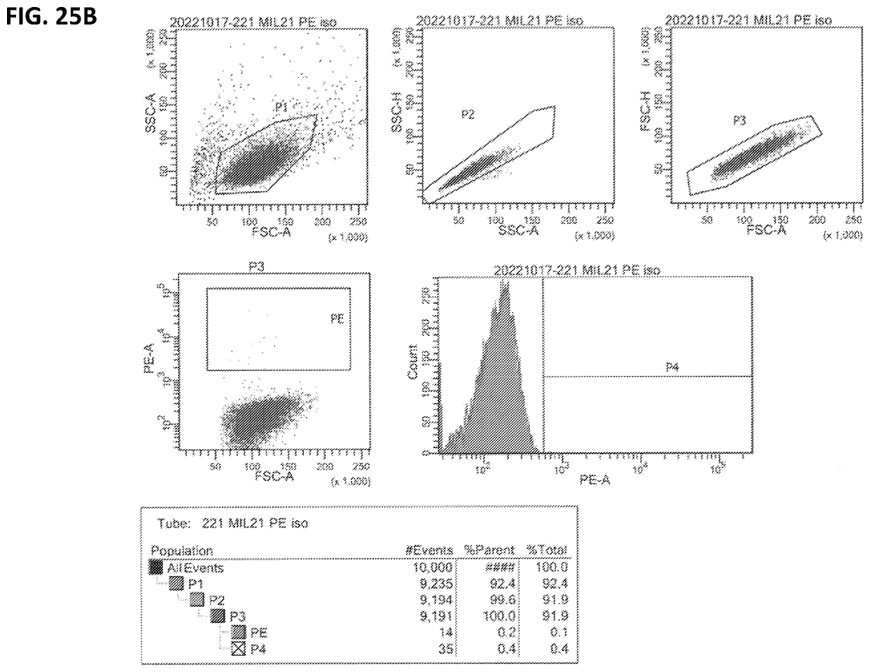

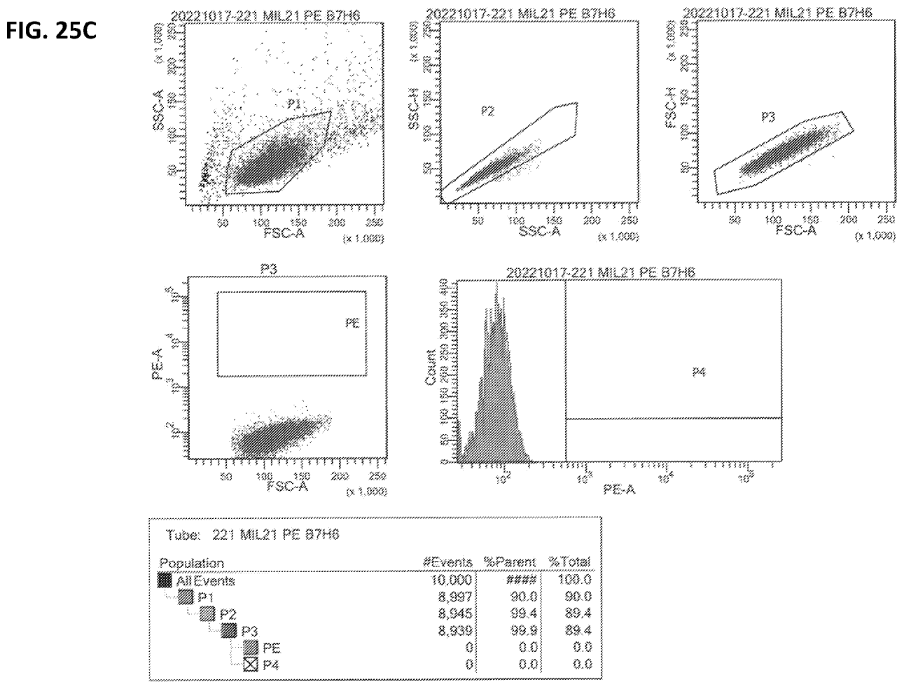

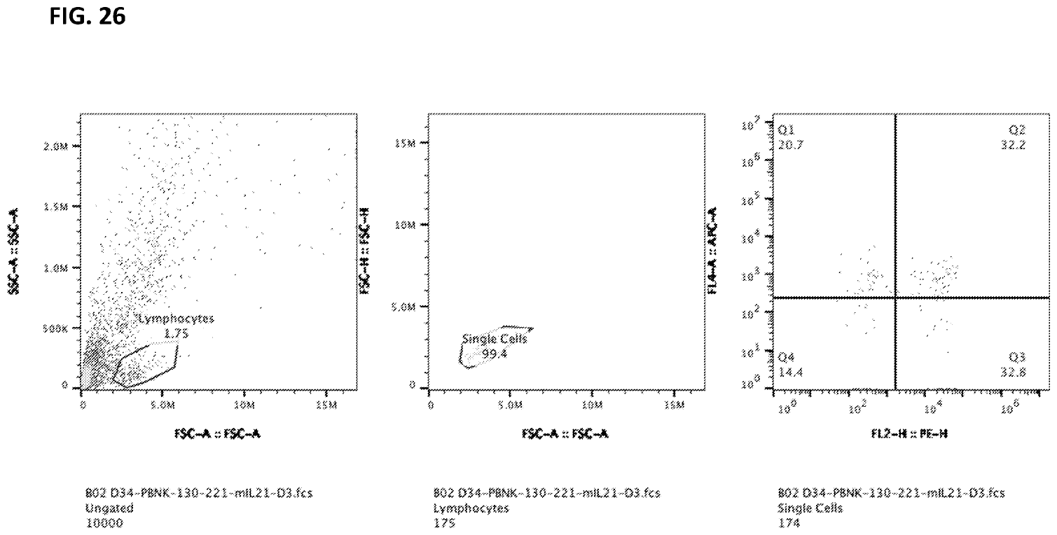

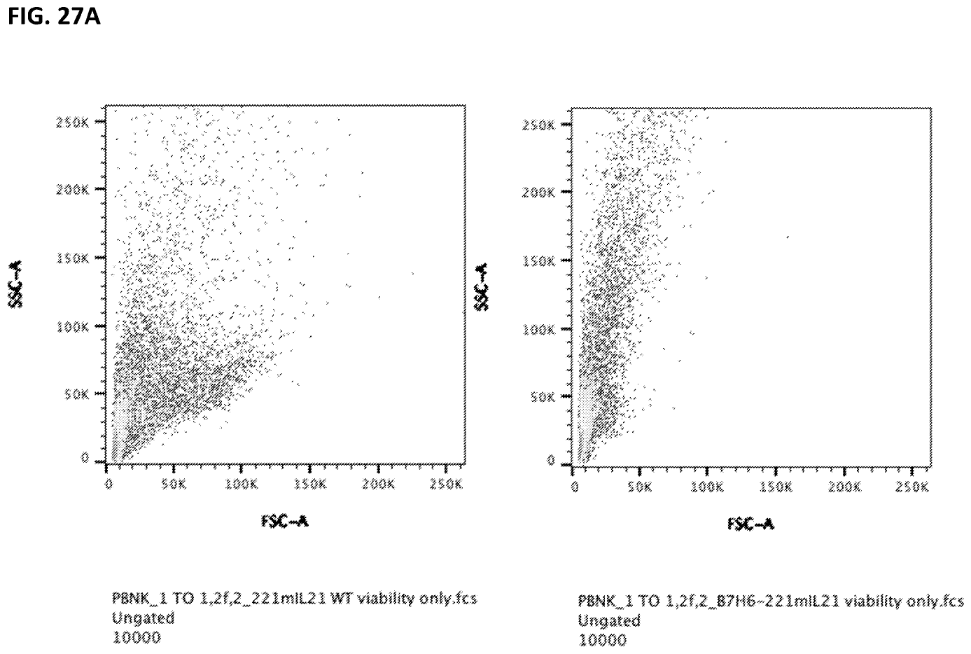

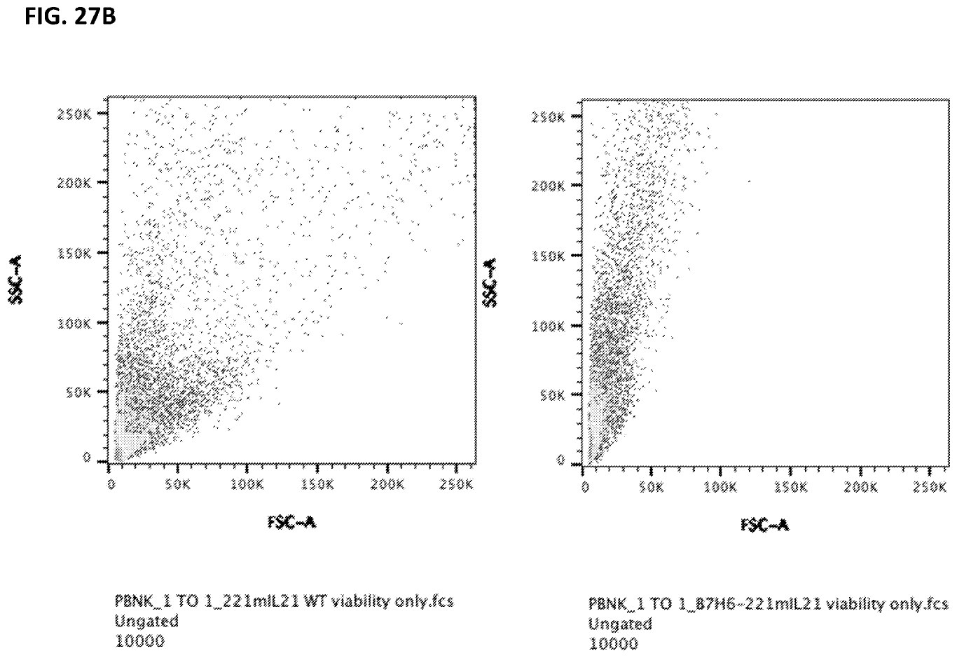

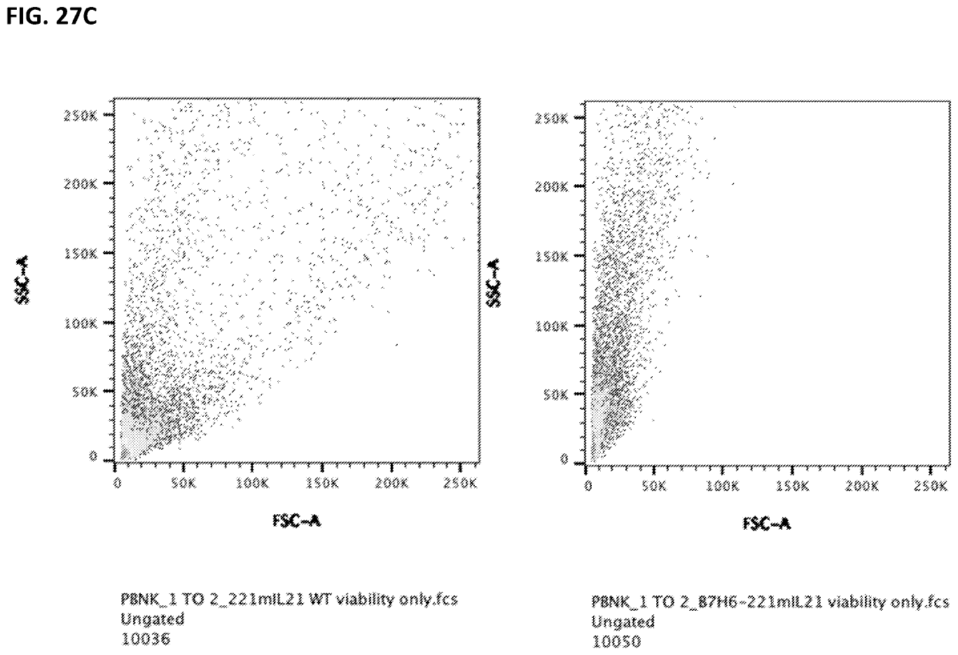

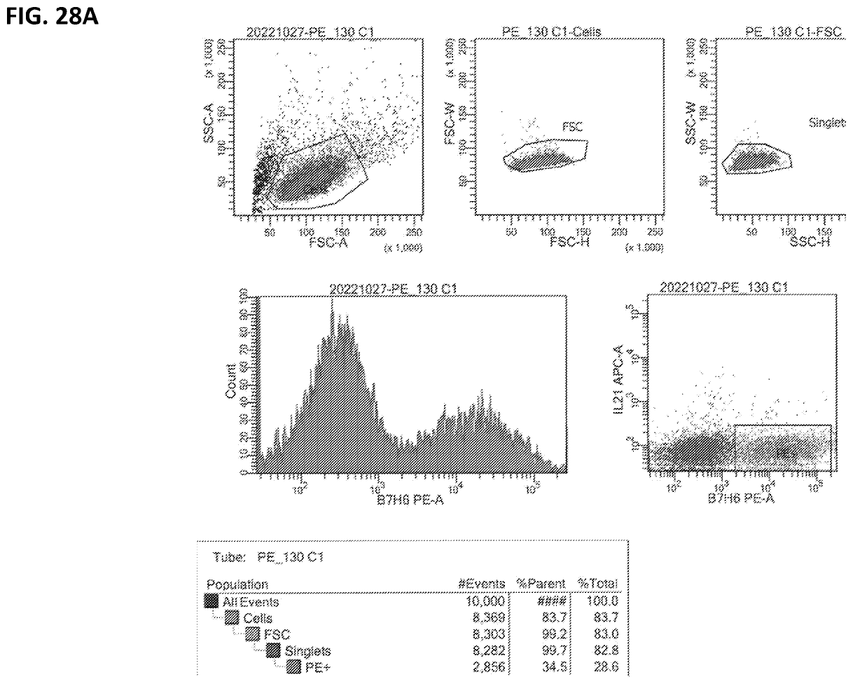

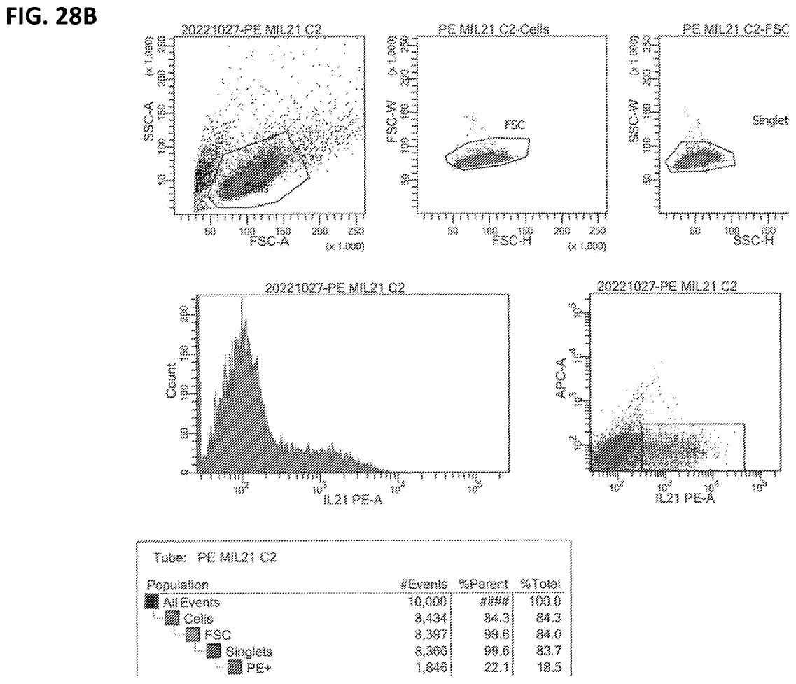

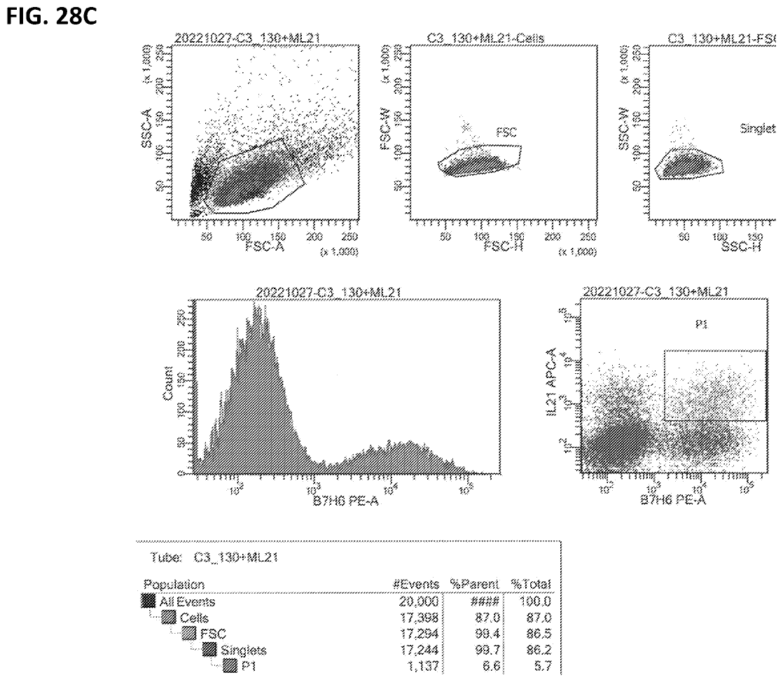

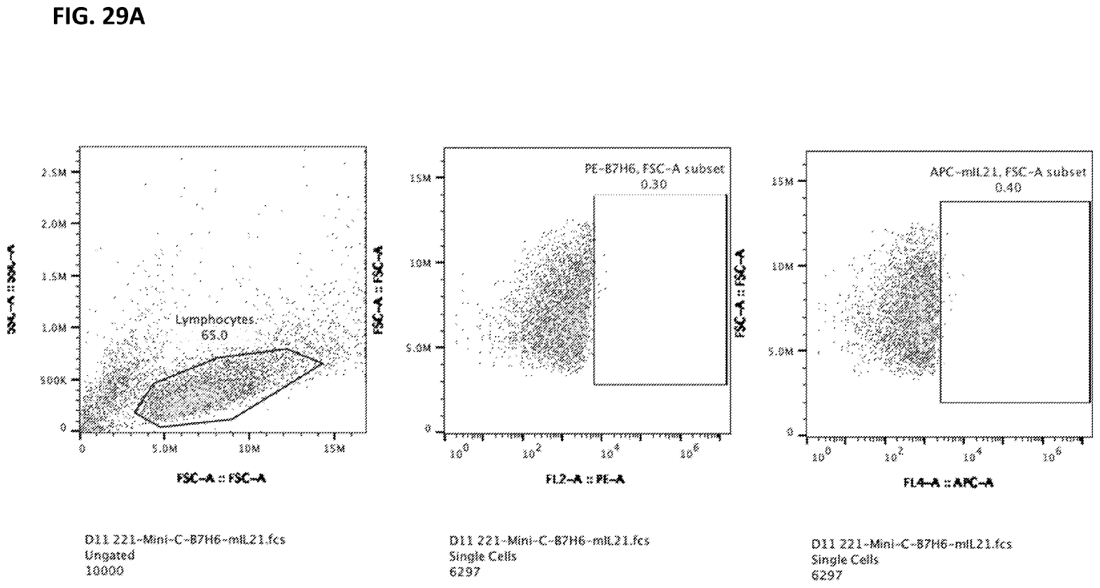

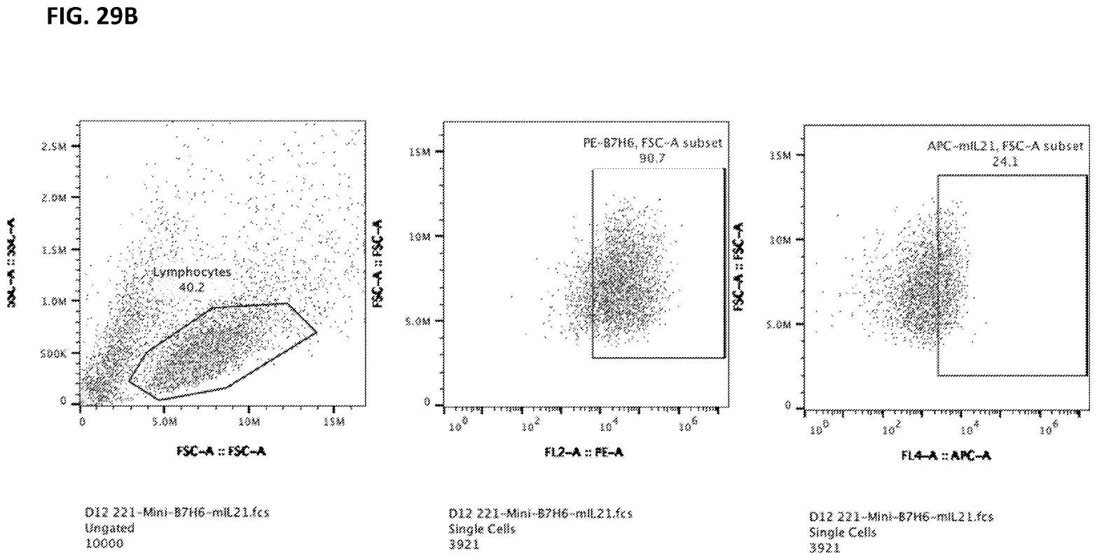

The effect of different amounts of exosomes (0.1 mg or 1 mg) was assessed and compared to the FC system. As shown in FIG. 21, a higher amount of exosomes (1 mg) has better NK expansion compared to a lower amount of exosomes (0.1 mg). The expanded NK cells also persistent longer than lower amount of exosomes.

### Example 9

### Expansion of NK Cells with 721.221-mIL-21 Cells Co-Expressing B7-H6

A construct for expression of B7-H6 in 721.221 cells was prepared using PCR primer set 230-18ntSFG Car_20ntB7H6_F (GGTGTCCAGTGCGTCGACatgacgtggagggctgccgc; SEQ ID NO: 13) and 230-18ntFlag_33ntB7H6_R (ATCCTTGTAATCTCTAGActgtaggggtaacagtaaagttgggggttg; SEQ ID NO: 14) and using the origene plasmid (RC232774), which includes 1362 bp B7H6 cDNA sequence, as the PCR template. The B7H6 PCR product was inserted into the SFG vector by In-fusion HD cloning kit (Takara Biol.) to make the plasmid CHC130-pSFG-B7H6-Flag_puroR. Retrovirus supernatant containing the B7H6 gene was harvested by co-transfecting the 293T cells with the pRDF/Pepam3/and CHC130-pSFG-B7H6-flag_puroR for 48 hrs and 0.5 million of 221-mIL21 cells was transduced in a 24-well plate for 48 hrs followed by cell sorting. The sorted cells were further cultured for at least 7 days, the expression of mIL21 and B7H6 were verified by flow cytometry.

TABLE 3
Culture pH and glucose with varying ratios
of PBMC to modified 721.221 cells

| | pH | Glucose mg/dL |
|---|---|---|
| 721.221-mIL21 | | |
| 1 to 2 | 7.1 | nd |
| 1 to 1 | 7.27 | nd |
| 1 to 0.5 | 7.42 | nd |
| 721.221-mIL21-B7H6 | | |
| 1 to 2 | 6.99 | 309 |
| 1 to 1 | 7.11 | 336 |
| 1 to 0.5 | 7.23 | 409 |

nd, not determined

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method of expanding a population of immune cells, comprising:
contacting the population of immune cells with an exosome isolated from a population of 721.221 cells transduced or transfected with a nucleic acid encoding membrane-bound IL-21 (mIL-21), wherein the contacting occurs under conditions sufficient for cell expansion;
thereby expanding the population of immune cells.

2. A method of producing an isolated exosome, comprising:
isolating the exosome from a supernatant of a culture of a population of 721.221 cells transduced or transfected with a nucleic acid encoding mIL-21;
thereby producing the isolated exosome.

3. The method of claim 1, wherein the population of 721.221 cells is further transduced or transfected with a nucleic acid encoding B7-H6.

4-5. (canceled)

6. A method of treating a cancer or an infectious or immune disease, comprising:
administering a population of immune cells expanded according to the method of claim 1 to a subject with the cancer or the infectious or immune disease;
thereby treating the cancer or the infectious or immune disease.

7. A method of treating a cancer or an infectious or immune disease, comprising:
contacting a population of immune cells with the isolated exosome produced according to the method of claim 2, thereby expanding the population of immune cells; and
administering the expanded population of immune cells to a subject with the cancer or the infectious or immune disease;
thereby treating the cancer or the infectious or immune disease.

8. The method of claim 1, wherein:
the immune cell is a natural killer (NK) cell, a T cell, a macrophage, or an natural killer T (NKT) cell;
the population of immune cells is contacted with the isolated exosome for 1-40 days;
the population immune cells is contacted with the isolated exosome is in the absence of feeder cells and/or
further comprising treating the population of immune cells with at least one cytokine.

9-11. (canceled)

12. The method of claim 8, wherein the at least one cytokine comprises at least one interleukin and/or comprises interleukin-15 (IL-15), interleukin-2 (IL-2), or a combination thereof.

13. (canceled)

14. The method of claim 1, further comprising;
treating the population of 721.221 cells with one or more toll-like receptor (TLR) ligands, prior to isolating the exosome; and/or
isolating mIL-21 positive 721.221 cells from the population of 721.221 cells and/or isolating B7-H6 positive 721.221 cells from the population of 721.221 cells, prior to isolating the exosome.

15-19. (canceled)

20. The method of claim 1, wherein;
(a) the nucleic acid encoding the mIL-21:
comprises a nucleic acid sequence comprising at least 90% or at least 95% sequence identity to SEQ ID NO: 1; or
encodes an amino acid sequence comprising at least 90% or at least 95% sequence identity to SEQ ID NO: 2; and/or
(b) the nucleic acid encoding the B7-H6:
comprises a nucleic acid sequence comprising at least 90% or at least 95% sequence identity to SEQ ID NO: 11; or
encodes an amino acid sequence comprising at least 90% or at least 95% sequence identity to SEQ ID NO: 12.

21. (canceled)

22. The method of claim 1, wherein the population of 721.221 cells is transduced with the nucleic acid encoding mIL-21, B7-H6, or both using a viral vector, a retroviral vector, a Moloney murine leukemia virus (MoMLV) vector, or an SFG retroviral vector.

23-25. (canceled)

26. The method of claim 22, wherein the viral vector:
comprises a nucleic acid sequence encoding the IgG1 CH2-CH3 domain, T cell surface glycoprotein CD3 zeta chain (CD3ζ), 4-1BB, CD28, or a combination thereof;
comprises at least 90% or 95% sequence identity to SEQ ID NO: 3; and/or
encodes an amino acid sequence comprising at least 90% or 95% sequence identity to SEQ ID NO: 10.

27-28. (canceled)

29. The method of claim 1, wherein the population of 721.221 cells is further transduced or transfected with a nucleic acid encoding an additional heterologous cytokine, activating receptor ligand, TLR ligand, or receptor thereof, and/or IL-15Rα.

30. The method of claim 1, wherein the population of immune cells:
is from peripheral blood, cord blood, ascites, menstrual blood, or bone marrow;
is autologous to the subject with cancer or immune disease;
comprises peripheral blood mononuclear cells (PBMCs) or purified NK cells; and/or
comprises chimeric antigen receptor (CAR)-modified immune cells.

31-33. (canceled)

34. The method of claim 30, wherein the CAR-modified immune cells comprise;
CAR-modified T cells, CAR-modified NK cells, CAR-modified macrophages, or CAR-modified NKT cells; and/or
CD19 CAR-modified NK cells or CD147 CAR-modified NK cells.

35. (canceled)

36. The method of claim 6, wherein:
the cancer or immune disease comprises an autoimmune disease, a transplant rejection, a sarcoma, a neuroblastoma, a solid tumor, or a blood cancer; or
the infectious disease comprises human immunodeficiency virus (HIV), hepatitis B virus (HBV), hepatitis C virus (HCV), tuberculosis (TB), or malaria.

37-38. (canceled)

39. A modified 721.221 cell expressing membrane-bound IL-21 (mIL-21) and B7-H6.

40. The modified 721.221 cell of claim 39, wherein;
(a) the mIL-21 comprises:
an amino acid sequence comprising at least 90% or at least 95% sequence identity to SEQ ID NO: 2; and/or
a nucleic acid comprising at least 90% or at least 95% sequence identity to SEQ ID NO: 1; and/or
(b) the B7-H6 comprises:
a nucleic acid sequence comprising at least 90% or at least 95% sequence identity to SEQ ID NO: 11; and/or
an amino acid sequence comprising at least 90% or at least 95% sequence identity to SEQ ID NO: 12; and/or
the cell further comprises a nucleic acid sequence encoding IgG1 CH2-CH3 domain, T cell surface glycoprotein CD3 zeta chain (CD3C), 4-1BB, CD28, or a combination thereof.

41. (canceled)

42. The modified 721.221 cell of claim 39, wherein the mIL-21, the B7-H6, or both, is expressed in the 721.221 cell using a viral vector.

43-45. (canceled)

46. The modified 721.221 cell of claim 39, wherein the cell further expresses:
at least one additional heterologous interleukin and/or interleukin receptor; and/or
at least one additional heterologous cytokine, activating receptor ligand, TRL ligand, or receptor thereof.

47-48. (canceled)

49. A method of producing the modified 721.221 cell of claim 39, comprising:
transducing or transfecting a population of 721.221 cells with a nucleic acid encoding mIL-21 and a nucleic acid encoding B7-H6;
isolating the cells that express the mIL-21 and B7-H6; and
irradiating the isolated cells,
thereby producing the modified 721.221 cell.

50. (canceled)

51. The method of claim 49, wherein:
the mIL-21 comprises an amino acid sequence comprising at least 90% or at least 95% sequence identity to SEQ ID NO: 2; and/or
the nucleic acid encoding the mIL-21 comprises a nucleic acid sequence comprising at least 90% or at least 95% sequence identity to SEQ ID NO: 1; and/or
the B7-H6 comprises an amino acid sequence comprising at least 90% or at least 95% sequence identity to SEQ ID NO: 12; and/or
the nucleic acid encoding the B7-H6 comprises a nucleic acid sequence comprising at least 90% or at least 95% sequence identity to SEQ ID NO: 11.

52. A method of expanding a population of natural killer (NK) cells or T cells, comprising contacting a population of cells with the modified 721.221 cells of claim 39 and at least one cytokine for 1-40 days under conditions sufficient for cell expansion.

53. The method of claim 52, wherein the population of cells:
is from peripheral blood, cord blood, ascites, menstrual blood, or bone marrow;
comprises peripheral blood mononuclear cells (PBMCs); and/or
comprises chimeric antigen receptor (CAR)-modified cells.

54-57. (canceled)

58. The method of claim 52, wherein the at least one cytokine comprises at least one interleukin, optionally interleukin-15 (IL-15) and/or interleukin-2 (IL-2).

59-61. (canceled)

62. A method of treating a cancer or an infectious or immune disease, comprising administering natural killer (NK) cells or T cells produced by the method of claim 52 to a subject with cancer or an infectious or immune disease, thereby treating the cancer or infectious or immune disease.

63. A method of treating a cancer or an infectious or immune disease, comprising:
contacting a population of cells with the modified 721.221 cells of claim 39 and at least one cytokine for at least 14-21 days, thereby producing natural killer (NK) cells or T cells; and
administering the NK cells or T cells to a subject with cancer or an infectious or immune disease, thereby treating the cancer or infectious or immune disease.

64. (canceled)

65. The method of claim 62, wherein the population of cells:
are from a subject with cancer or infectious or immune disease; and/or
comprise CAR-modified lymphocytes.

66-72. (canceled)


SEQUENCE LISTING (14 sequences)

SEQ ID NO: 1 - DNA, 462 bp, synthetic construct
  misc_feature 1..462: note = Synthetic nucleotide sequence
  source 1..462: mol_type = other DNA
atggagtttgggctgagctggctttttcttgtggctattttaaaaggtgtccagtgcgtcgaccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcc

SEQ ID NO: 2 - protein, 154 aa, synthetic construct
  REGION 1..154: note = Synthetic amino acid sequence
MEFGLSWLFLVAILKGVQCVDQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 3 - DNA, 1881 bp, synthetic construct
  misc_feature 1..1881: note = Synthetic nucleotide sequence
  source 1..1881: mol_type = other DNA
atggagtttgggctgagctggctttttcttgtggctattttaaaaggtgtccagtgcgtcgaccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccctgcggccgccgtacgtcaccgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttttgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 4 - DNA, 1566 bp, Homo sapiens
  source 1..1566: mol_type = unassigned DNA
ctgggcagcgctcgcccggggagtccagcggtgtcctgtggagctgccgccatggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgaaactcggggaaaccagcccagctaagtccggagtgaaggagcctctctgctttagctaaagacgactgagaagaggtgcaaggaagcgggctccaggagcaagctcaccaggcctctcagaagtcccagcaggatctcacggactgccgggtcggcgcctcctgcgcgagggagcaggttctccgcattcccatgggcaccacctgcctgcctgtcgtgccttggacccagggcccagcttcccaggagagaccaaaggcttctgagcaggatttttatttcattacagtgtgagctgcctggaatacatgtggtaatgaaataaaaaccctgccccgaatcttccgtccctcatcctaactttcagttcacagagaaaagtgacatacccaaagctctctgtcaattacaaggcttctcctggcgtgggagacgtctacagggaagacaccagcgtttgggcttctaaccaccctgtctccagctgctctgcacacatggacagggacctgggaaaggtgggagagatgctgagcccagcgaatcctctccattgaaggattcaggaagaagaaaactcaactcagtgccattttacgaatatatgcgtttatatttatacttccttgtctattatatctatacattatatattatttgtattttgacattgtaccttgtataaacaaaataaaacatctattttcaata

SEQ ID NO: 5 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 6 - DNA, 2333 bp, Homo sapiens
  source 1..2333: mol_type = unassigned DNA
gttgggactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgacgtcacatggagcacagaaatcaatgttagcagatagccagcccatacaagatcgttttcaactagtggccccactgtgtccggaattgatgggttcttggtctcactgacttcaagaatgaagccgcggaccctcgcggtgagtgttacagctcttaaggtggcgcatctggagtttgttccttctgatgttcggatgtgttcggagtttcttccttctggtgggttcgtggtctcgctggctcaggagtgaagctacagaccttcgcggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggatgcagctaatatacccagttggcccaaagcacctaacctatagttatataatctgactctcagttcagttttactctactaatgccttcatggtattgggaaccatagatttgtgcagctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaaaaaaaaaaaa

SEQ ID NO: 7 - protein, 135 aa, Homo sapiens
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - DNA, 822 bp, Homo sapiens
  source 1..822: mol_type = unassigned DNA
agttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 9 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 626 aa, synthetic construct
  REGION 1..626: note = Synthetic amino acid sequence
MEFGLSWLFLVAILKGVQCVDQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSLRPPYVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1362 bp, Homo sapiens
  source 1..1362: mol_type = unassigned DNA
atgacgtggagggctgccgcctccacgtgcgcggcgctcctgattctgctgtgggcgctgacgaccgaaggtgatctgaaagtagagatgatggcaggggggactcagatcacacccctgaatgacaatgtcaccatattctgcaatatcttttattcccaacccctcaacatcacgtctatgggtatcacctggttttggaagagtctgacgtttgacaaagaagtcaaagtctttgaattttttggagatcaccaagaggcattccgacctggagccattgtgtctccatggaggctgaagagtggggacgcctcactgcggctgcctggaatccagctggaggaagcaggagagtaccgatgtgaggtggtggtcacccctctgaaggcacagggaacagtccagcttgaagttgtggcttccccagccagcagattgttgctggatcaagtgggcatgaaagagaatgaagacaaatatatgtgtgagtcaagtgggttctacccagaggctattaatataacatgggagaagcagacccagaagtttccccatcccatagagatttctgaggatgtcatcactggtcccaccatcaagaatatggatggcacatttaatgtcactagctgcttgaagctgaactcctctcaggaagaccctgggactgtctaccagtgtgtggtacggcatgcgtccttgcatacccccttgaggagcaactttaccctgactgctgctcggcacagtctttctgaaactgagaagacagataatttttccattcattggtggcctatttcattcattggtgttggactggttttattaattgttttgattccttggaaaaagatatgtaacaaatcatcttcagcctatactcctctcaagtgcattctgaaacactggaactcctttgacactcagactctgaagaaagagcacctcatattcttttgcactcgggcatggccgtcttaccagctgcaggatggggaggcttggcctcctgagggaagtgttaatattaatactattcaacaactagatgttttctgcagacaggagggcaaatggtccgaggttccttatgtgcaagccttctttgccttgcgagacaacccagatctttgtcagtgttgtagaattgaccctgctctcctaacagttacatcaggcaagtccatagatgataattccacaaagtctgagaaacaaacccctagggaacactcggatgcagttccggatgccccaatccttcctgtctcccctatctgggaacctcctccagccacaacatcaacaactccagttctatcctcccaacccccaactttactgttacccctacag

SEQ ID NO: 12 - protein, 454 aa, Homo sapiens
MTWRAAASTCAALLILLWALTTEGDLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFSIHWWPISFIGVGLVLLIVLIPWKKICNKSSSAYTPLKCILKHWNSFDTQTLKKEHLIFFCTRAWPSYQLQDGEAWPPEGSVNINTIQQLDVFCRQEGKWSEVPYVQAFFALRDNPDLCQCCRIDPALLTVTSGKSIDDNSTKSEKQTPREHSDAVPDAPILPVSPIWEPPPATTSTTPVLSSQPPTLLLPLQ

SEQ ID NO: 13 - DNA, 38 bp, synthetic construct
  source 1..38: mol_type = other DNA
ggtgtccagtgcgtcgacatgacgtggagggctgccgc

SEQ ID NO: 14 - DNA, 48 bp, synthetic construct
  source 1..48: mol_type = other DNA
atccttgtaatctctagactgtaggggtaacagtaaagttgggggttg